SEQUENCE LISTING (6 sequences)

SEQ ID NO: 1 - DNA, 21 bp, Artificial
  Synthetic construct
gggtgccaaagtagaactgcg

SEQ ID NO: 2 - DNA, 22 bp, Artificial
  Synthetic construct
gacagaataaaacgcacgggtg

SEQ ID NO: 3 - DNA, 24 bp, Artificial
  Synthetic construct
ttcagccaagcctatgtagaaagc

SEQ ID NO: 4 - DNA, 19 bp, Artificial
  Synthetic construct
gccaaagtagaactgcgtt

SEQ ID NO: 5 - DNA, 19 bp, Artificial
  Synthetic construct
gcgctagacaagttcctta

SEQ ID NO: 6 - DNA, 19 bp, Artificial
  Synthetic construct
gcaccagttcagcaagact

(19) United States
(12) Patent Application Publication
(10) Pub. No.: US 2010/0254996 A1
(43) Pub. Date: Oct. 7, 2010

(54) SYNERGISTIC TREATMENT OF CELLS THAT EXPRESS EPHA2 AND ERBB2

(75) Inventors: Dana Brantley-Sieders, Nashville, TN (US); Jin Chen, Nashville, TN (US); Elizabeth Bruck-Heimer, Phoenix, AZ (US); Dowdy Jackson, Frederick, MD (US)

Correspondence Address: MEDIMMUNE, LLC;Patrick Scott Alban, ONE MEDIMMUNE WAY, GAITHERSBURG, MD, 20878, US

(73) Assignees: MEDIMMUNE, LLC, Gaithersburg, MD (US); VANDERBILT UNIVERSITY, Nashville, TN (US)

(21) Appl. No.: 12/665,176

(22) PCT Filed: Jun. 16, 2008
(86) PCT No.: PCT/US08/67114

§ 371 Date: Jun. 21, 2010

Related U.S. Application Data
(60) Provisional application No. 60/929,212, filed on Jun. 18, 2007.

(51) Int. Cl. A61K 39/395; A61K 38/02; A61K 31/713; A61P 35/00

(52) U.S. Cl. 424/158.1; 514/19.2; 514/44 A; 424/178.1

(57) ABSTRACT

The present invention relates to methods of treating hyperproliferative cells that express EphA2 and ErbB2. The present invention further relates to methods of selecting patient populations for treatment methodologies.

### 2. CROSS REFERENCE TO RELATED APPLICATIONS

This application claims the benefit under 35 U.S.C. §119(e) of the following U.S. Provisional Application No. 60/929,212 filed Jun. 18, 2007. The priority application is hereby incorporated by reference herein in their entirety for all purposes.

### 1. STATEMENT AS TO RIGHTS TO INVENTIONS MADE UNDER FEDERALLY SPONSORED RESEARCH AND DEVELOPMENT

This invention was made, in part, with United States Government support under award numbers CA95004, CA114301, and CA1179151-02 from the National Institutes of Health, and award number W81XWH-05-01-0254 from the Department of Defense. The United States Government may have certain rights in the invention.

### 3. FIELD OF THE INVENTION

The present invention provides methods of treating hyperproliferative cells that express EphA2 and ErbB2. The present invention further provides methods of selecting patient populations for treatment methodologies.

### 4. BACKGROUND OF THE INVENTION

### 4.1 Cancer

A neoplasm, or tumor, is a neoplastic mass resulting from abnormal uncontrolled cell growth, which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading case of death in the United States and if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime. Current treatment options, such as surgery, chemotherapy and radiation treatment, are oftentimes either ineffective or present serious side effects.

The most life-threatening forms of cancer often arise when a population of tumor cells gains the ability to colonize distant and foreign sites in the body. These metastatic cells survive by overriding restrictions that normally constrain cell colonization into dissimilar tissues. For example, typical mammary epithelial cells will generally not grow or survive if transplanted to the lung, yet lung metastases are a major cause of breast cancer morbidity and mortality. Recent evidence suggests that dissemination of metastatic cells through the body can occur long before clinical presentation of the primary tumor. These micrometastatic cells may remain dormant for many months or years following the detection and removal of the primary tumor. Thus, a better understanding of the mechanisms that allow for the growth and survival of metastatic cells in a foreign microenvironment is critical for the improvement of therapeutics designed to fight metastatic cancer and diagnostics for the early detection and localization of metastases.

Cancer is a disease of aberrant signal transduction. Aberrant cell signaling overrides anchorage-dependent constraints on cell growth and survival (Rhim, et al., Critical Reviews in Oncogenesis 8:305, 1997; Patarca, Critical Reviews in Oncogenesis 7:343, 1996; Malik, et al., Biochimica et Biophysica Acta 1287:73, 1996; Callahan et al., Breast Cancer Res Treat 35:105, 1995). Tyrosine kinase activity is induced by ECM anchorage and indeed, the expression or function of tyrosine kinases is usually increased in malignant cells (Rhim, et al., Critical Reviews in Oncogenesis 8:305, 1997; Callahan et al., Breast Cancer Res Treat 35:105, 1995; Hunter, Cell 88:333, 1997). Based on evidence that tyrosine kinase activity is necessary for malignant cell growth, tyrosine kinases have been targeted with new therapeutics (Levitzki, et al., Science 267:1782, 1995; Kondapaka, et al., Molecular & Cellular Endocrinology 117:53, 1996; Fry, et al., Current Opinion in BioTechnology 6: 662, 1995). Unfortunately, obstacles associated with specific targeting to tumor cells often limit the application of these drugs. In particular, tyrosine kinase activity is often vital for the function and survival of benign tissues (Levitzki, et al., Science 267:1782, 1995). To minimize collateral toxicity, it is critical to identify and then target tyrosine kinases that are selectively overexpressed in tumor cells.

Malignant progression of solid tumors is a complex process that involves the activation of oncogenic signaling and downregulation of tumor suppressor pathways. In addition, modulation of the tumor microenvironment, for example through neovascularization, enhances tumor cell growth and survival, promoting invasion and metastatic spread [Reviewed in (Hahn and Weinberg, 2002; Hanahan and Weinberg, 2000; Vogelstein and Kinzler, 2004)]. Oncogenic conversion or overexpression of proto-oncogenes, such as those encoding cell surface receptor tyrosine kinases (RTKs) like the EGF receptor family member ErbB2, are frequently observed in human cancers and contribute to malignancy. Other pathways, such as p53 transcription factor/genome surveillance factor, negatively regulate growth and loss of these pathway components contributes to cancer [Reviewed in (Blume-Jensen and Hunter, 2001; Vogelstein and Kinzler, 2004)]. Recent evidence suggests that Eph RTKs contribute to tumor progression in several types of cancer both within the tumor parenchyma and within the stromal microenvironment, including endothelium [Reviewed in (Brantley-Sieders et al., 2004a; Brantley-Sieders and Chen, 2004); (Brantley-Sieders et al., 2005)].

### 4.1.1 Receptor Tyrosine Kinases

Receptor tyrosine kinases (RTKs) are transmembrane proteins which consist of an extracellular ligand binding domain and an intracellular domain with tyrosine kinase activity (Surawska et al., 2004, Cytokine Growth Factor Rev. 15:419-433). This family of proteins contains over fifty different members that are organized into at least nineteen different classes based on structural organization, and includes receptors for growth factors (e.g. EGF, PDGF, FGF) and insulin (Grassot et al, 2003, Nucl Acids Res., 31(1):353-358; Surawska et al., 2004, Cytokine Growth Factor Rev. 15:419-433). Class I RTK's comprise, for example, EGFR, ERBB2, ERBB3 and ERBB4; Class II RTK's comprise, for example, INSR, IRR and IG1R; Class III RTK's comprise, for example, PDGFa, PDGFb, Fms, Kit and Flt3; Class IV RTK's comprise, for example, FGFR1, FGFR2, FGFR3, FGFR4 and BFR2; Class V RTK's comprise Flt1, Flt2 and Flt4; Class VI RTK's comprise EphA1-EphA8 and EphB1-EphB6; Class VII RTK's comprise TrkA, TrkB and TrkC (Grassot et al., 2003, Nucl Acids Res., 31(1):353-358). Autophosphorylation of the tyrosine residues in the intracellular (cytosolic) domain is induced by ligand binding to the extracellular binding domain, which in turn leads to the formation of signaling complexes and activation of downstream signal transduction cascades (Surawska et al., 2004, Cytokine Growth Factor Rev. 15:419-433).

### 4.1.2 EphA2

The Eph RTK family is the largest family of RTKs identified in the genome, with at least 15 receptors and 9 ligands identified in vertebrates [Reviewed in (Brantley-Sieders and Chen, 2004; Murai and Pasquale, 2003)]. The family is subdivided into class A and class B based on homology and binding affinity for two distinct types of membrane-anchored ephrin ligands. Class B receptors generally bind to class B ephrins that are attached to the cell membrane by a transmembrane-spanning domain, while A class receptors normally interact with to glycosyl-phosphatidylinositol (GPI)-linked class A ephrins, though some interclass binding has been observed for certain family members [Reviewed in (Brantley-Sieders and Chen, 2004; Murai and Pasquale, 2003)]. Receptors in the Eph subfamily typically have a single kinase domain and an extracellular region containing a Cys-rich domain and 2 fibronectin type III repeats. The Eph receptors, and their membrane bound ephrin ligands are important mediators of cell-cell communication regulating cell attachment, shape, and mobility. Eph RTK signaling events control multiple aspects of embryonic development, particularly in the nervous system (reviewed in Kullander et al., 2002, Nat. Rev. Mol. Cell Biol. 3:473 and Mamling et al., 2002, Trends Biochem Sci 27:514-520. These molecules function during embryogenesis to regulate angiogenic remodeling processes, axon guidance, and tissue boundary formation [Reviewed in (Pasquale, 2005; Poliakov et al., 2004)].

Many members of the Eph receptors have been identified as important markers and/or regulators of the development and progression of cancer (see for example Thaker et al., 2004, Clin. Cancer Res. 10:5145; Fox B P et al., 2004, Biochem. Biophys. Res. Commun. 318:882; Nakada et al., 2004, Cancer Res. 64:3179; Coffman et al., 2003, Cancer Res. 63:7907; also reviewed in Dodelet et al., 2000, Oncogene 19:5614). Of the Eph receptors known to be involved in cancer the role and expression patterns of EphA2 and EphA4 are among the best characterized.

EphA2 (epithelial cell kinase) is a 130 kDa member of the Eph family of receptor tyrosine kinase (Zantek N. et al, 1999, Cell Growth Differ. 10:629-38; Lindberg R. et al., 1990, Mol. Cell. Biol. 10:6316-24). Although the function of EphA2 is still being studied, but it has been suggested to regulate proliferation, differentiation, and barrier function of colonic epithelium (Rosenberg I. et al., 1997, Am. J. Physiol. 273:G824-32), vascular network assembly, endothelial migration, tumor angiogenesis, and angiogenesis (D. M. Brantley, J. Caughron, D. Hicks, A. Pozzi, J. C. Ruiz, and J. Chen (2004) EphA2 receptor tyrosine kinase regulates endothelial cell migration and assembly via PI3K-mediated Rac1 activation J. Cell Sci. 117:2037-3049, D. Brantley, N. Cheng, E. Thompson, Q. Lin, R. A. Brekken, P. E. Thorpe, R. S. Muraoka, D. Cerretti, A. Pozzi, D. Jackson, C. Lin, and J. Chen. (2002) Soluble EphA receptor inhibit tumor progression and angiogenesis in vivo. Oncogene 21:7011-7026, N. Cheng, D. Brantley, H. Liu, A. Lin, M. Enriquiz, D. Ceretti, N. Gale, G. Yancouplous, T. Daniel, and J. Chen. (2002) Blockade of EphA receptor tyrosine activation inhibits VEGF-dependent angiogenesis. Mol. Cancer Res. (formerly Cell Growth and Differentiation) 1:2-11, N. Cheng, D. Brantley, H. Liu, W. Fanslow, D. Cerretti, D. Jackson, and J. Chen. (2003) Inhibition of VEGF-dependent multi-stage carcinogenesis by soluble EphA receptors. Neoplasia 5:445-456, D. M. Brantley-Sieders, W. B. Fang, D. Hicks, T. Koyama, Y. Shyr, and J. Chen. (2005) Impaired tumor microenvironment in EphA2-deficient mice inhibits tumor angiogenesis and metastatic progression. FASEB J. 19:1884-6, D. M. Brantley-Sieders, W. B. Fang, Y. Hwang, and J. Chen. (2006) Ephrin-A1 facilitates tumor metastasis via an angiogenic-dependent mechanism in vivo. Cancer Res. 66:10315-10324), nervous system segmentation and axon pathfinding (Bovenkamp D. and Greer P., 2001, DNA Cell Biol. 20:203-13), tumor neovascularization (Ogawa K. et al., 2000, Oncogene 19:6043-52), and cancer metastasis (International Patent Publication Nos. WO 01/9411020, WO 96/36713, WO 01/12840, WO 01/12172).

The natural ligand of EphA2 is Ephrin A1 (Eph Nomenclature Committee, 1997, Cell 90(3):403-4; Gale, et al., 1997, Cell Tissue Res. 290(2): 227-41). The EphA2 and Ephrin A1 interaction is thought to help anchor cells on the surface of an organ and also down regulate epithelial and/or endothelial cell proliferation by decreasing EphA2 expression through EphA2 autophosphorylation (Lindberg et al., 1990). Under natural conditions, the interaction helps maintain an epithelial cell barrier that protects the organ and helps regulate over proliferation and growth of epithelial cells. However, there are disease states that prevent epithelial cells from forming a protective barrier or cause the destruction and/or shedding of epithelial and/or endothelial cells and thus prevent proper healing from occurring.

EphA2 is expressed in adult epithelia, where it is found at low levels and is enriched within sites of cell-cell adhesion (Zantek, et al, 1999, Cell Growth & Diff 10:629; Lindberg, et al., 1990, Mol & Cell Biol 10: 6316). This subcellular localization is important because EphA2 binds EphrinsA1 to A5 that are anchored to the cell membrane (Eph Nomenclature Committee, 1997, Cell 90:403; Gale, et al., 1997, Cell & Tissue Res 290: 227). The primary consequence of ligand binding is EphA2 autophosphorylation (Lindberg, et al., 1990). However, unlike other receptor tyrosine kinases, EphA2 retains enzymatic activity in the absence of ligand binding or phosphotyrosine content (Zantek, et al., 1999). EphA2 and ephrin-A1 are upregulated in the transformed cells of a wide variety of tumors including breast, prostate, colon, lung, kidney, skin, and esophageal cancers (Ogawa, et al., 2000, Oncogene 19:6043; Zelinski, et al., 2001, Cancer Res 61:2301; Walker-Daniels, et al., 1999, Prostate 41:275; Easty, et al., 1995, Int J Cancer 60: 129; Nemoto, et al., 1997, Pathobiology 65:195; Hess et al., 2001, Cancer Res 61(8): 3250-5).

More recently, members of this RTK family, including EphA2, have been linked to tumor progression and neovascularization [Reviewed in (Brantley-Sieders et al., 2004)]. Increasing evidence suggests that EphA2 expression may be causally related to tumor progression. EphA2 receptor tyrosine kinase overexpression has been observed in several models of cancer, including primary and transplanted rodent tumors, human tumor xenografts, and primary human tumor biopsies [Reviewed in (Brantley-Sieders et al., 2004; Brantley-Sieders and Chen, 2004; Ireton and Chen, 2005)]. Experimentally induced overexpression of EphA2 resulted in malignant transformation of non-transformed MCF10A breast cells and enhanced malignancy of pancreatic carcinoma cells (Duxbury et al., 2004; Zelinski et al., 2001). Conversely, siRNA-mediated inhibition of EphA2 expression impaired malignant progression of pancreatic, ovarian and mesothelioma tumor cell lines, and overexpression of dominant-negative EphA2 constructs suppressed growth and metastasis of 4T1 metastatic mouse mammary adenocarcinoma cells in vivo (Duxbury et al., 2004; Landen et al., 2005; Nasreen et al., 2006, Fang, 2005). EphA-Fc receptor proteins that disrupt endogenous receptor activation significantly inhibited growth and neovascularization of tumors in vivo (Brantley et al., 2002; Cheng et al., 2003; Dobrzanski et al., 2004). Coupled with the observation that EphA2 receptor signaling induces phosphorylation and activation of the pro-proliferative p42/44 mitogen activated protein kinase (MAPK) family member Erk in tumor cell lines (Pratt and Kinch, 2002; Pratt and Kinch, 2003), these data suggest EphA2 receptor functions as an oncogene.

Other evidence, however, suggests that EphA2 may function as a tumor suppressor. EphA2-deficient gene-trap mice displayed increased susceptibility to chemical carcinogen-induced skin cancer compared to control littermates, along with increased tumor cell proliferation and elevated phosphorylation of Erk (Guo et al., 2006). Stimulation of EphA receptors with soluble ephrin-A1-Fc ligand reduced Erk phosphorylation in tumor cell lines, fibroblasts, and primary aortic endothelial cells, and suppressed growth of primary keratinocytes and prostate carcinoma cells (Guo et al., 2006; Macrae et al., 2005; Miao et al., 2001). Macrae et al. also reported that treatment of human breast cancer cell lines with ephrin-A1-Fc, which stimulated EphA2 phosphorylation, attenuated EGF mediated phosphorylation of Erk and inhibited transformation of NIH3T3 cells expressing v-erbB2 (Macrae et al., 2005). In addition, EphA2 was reported to be a transcriptional target of the tumor suppressor p53 (Dohn et al., 2001; Jin et al., 2006; Yang et al., 2006; Zhang et al., 2003). Overexpression in lung and breast cancer cell lines negatively regulated proliferation and induced apoptosis (Dohn et al., 2001; Jin et al., 2006). These data suggest that EphA2 functions as a tumor suppressor.

### 4.1.3 HER Family of RTK's

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2).

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-α), by the same tumor cells resulting in receptor activation by an autocrine stimulatory pathway (Baselga and Mendelsohn, Pharmac. Ther., 64:127-154 (1994)). Monoclonal antibodies directed against the EGFR or its ligands, TGF-α and EGF, have been evaluated as therapeutic agents in the treatment of such malignancies. See, e.g., Baselga and Mendelsohn; Masui et al., Cancer Research, 44:1002-1007 (1984); and Wu et al., J. Clin. Invest., 95:1897-1905 (1995).

The second member of the HER family, p185^{neu} (also known as HER2 and ErbB2), was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235:177-182 (1987); Slamon et al., Science, 244:707-712 (1989); and U.S. Pat. No. 4,968,603). To date, no point mutation analogous to that in the neu proto-oncogene has been reported for human tumors.

Overexpression of HER2 (frequently but not uniformly due to gene amplification) has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet, 1:765-767 (1986); Fukushige et al., Mol Cell Biol., 6:955-958 (1986); Guerin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421-425 (1990); Kern et al., Cancer Res., 50:5184 (1990); Park et al., Cancer Res., 49:6605 (1989); Zhau et al., Mol. Carcinog., 3:254-257 (1990); Aasland et al., Br. J. Cancer, 57:358-363 (1988); Williams et al., Pathobiology, 59:46-52 (1991); and McCann et al., Cancer, 65:88-92 (1990). HER2 may be overexpressed in prostate cancer (Gu et al., Cancer Lett., 99:185-9 (1996); Ross et al., Hum. Pathol., 28:827-33 (1997); Ross et al., Cancer, 79:2162-70 (1997); and Sadasivan et al., J. Urol., 150:126-31 (1993)).

HER2 amplification/overexpression is an early event in breast cancer that is associated with aggressive disease and poor prognosis. HER2 gene amplification is found in 20-25% of primary breast tumors (Slamon et al., Science, 244:707-12 (1989); Owens et al., Breast Cancer Res Treat, 76:S68 abstract 236 (2002)). HER2 positive disease correlates with decreased relapse-free and overall survival (Slamon et al., Science, 235:177-82 (1987); Pauletti et al., J. Clin Oncol, 18:3651-64 (2000)). Amplification of the HER2 gene is associated with significantly reduced time to relapse and poor survival in node-positive disease (Slamon et al. (1987); Pauletti et al. (2000)) and poor outcome in node-negative disease (Press et al., J. Clin Oncol, 1997; 15:2894-904 (1997); Pauletti et al. (2000)).

Given the roles that the RTK's play in the onset and progression of hyperproliferative diseases such as cancer, there is a pronounced need not only for methods of treating these disorders, but also for methods of selecting candidate populations for tailored therapies.

Citation or discussion of a reference herein shall not be construed as an admission that such is prior art to the present invention.

### 5. SUMMARY OF THE INVENTION

In one embodiment, the invention provides a method of reducing proliferation of hyperproliferative cells, said method comprising: a) identifying a population of hyperproliferative cells that express both EphA2 and ErbB2; and b) administering an agent that targets EphA2. In another embodiment, the invention provides a method of reducing proliferation of hyperproliferative cells that express both EphA2 and ErbB2 comprising administering an agent that targets EphA2. In a further embodiment, the invention provides a method of reducing proliferation of hyperproliferative cells that express both EphA2 and ErbB2 comprising administering an agent that targets EphA2, further comprising administering an agent that targets ErbB2.

In yet a further embodiment, the invention provides a method of treating a cancer patient, said method comprising: (a) determining the expression level, presence, or amount of EphA2 and ErbB2 in said patient's cancer cells; (b) determining whether the expression level or amount assessed in step (a) is above or below a certain quantity that is associated with an increased or decreased clinical benefit to a patient; and (c) administering an anti-EphA2 and/or an anti-ErbB2 targeting agent if it is determined that said patient's cancer cells express both EphA2 and ErbB2.

In another embodiment, the invention provides a method of prescreening a patient population for treatment with an anti-EphA2 and/or an anti-ErbB2 agent, said method comprising: (a) determining the expression level, presence, or amount of EphA2 and ErbB2 in said patient's cancer cells; (b) determining whether the expression level or amount assessed in step (a) is above or below a certain quantity that is associated with an increased or decreased clinical benefit to a patient.

### 6. BRIEF DESCRIPTION OF THE FIGURES

For the purpose of illustrating the invention, there are depicted in the drawings certain embodiments on the invention. However, the invention is not limited to the precise arrangements and instrumentalities of the embodiments depicted in the drawings.

FIG. 1. EphA2-deficiency reduces mammary tumorigenesis, metastasis, proliferation, and vascularity MMTV-Neu mice. (A) Mammary epithelial hyperplasia and tumor formation were less frequent in MMTV-Neu/EphA2 −/− female mice relative to heterozygous (+/−) and wild-type (+/+) control females that were analyzed 8 months (8 mo.) after birth. The frequency of tumor formation and lung metastasis was also reduced in MMTV-Neu/EphA2 −/− female mice analyzed 1 year after birth (1 yr.) relative to controls. (B) A significant reduction in the number of surface lung lesions was also observed for MMTV-Neu/EphA2 −/− females relative to controls (p<0.05; single factor ANOVA). Data are represented as mean±SEM. (C) Whole-mount hematoxylin staining of number 4 inguinal mammary glands collected from EphA2 +/+, +/−, and −/− MMTV-Neu female transgenic animals 8 months after birth reveals diminished hyperplasia in EphA2 −/− glands relative to controls. The upper left panel shows a +/+ gland with pervasive epithelial hyperplasia, and the center panel shows a +/− gland with a focal hyperplasia. * Indicates the inguinal lymph node. Hematoxylin and eosin-stained sections from contralateral inguinal mammary glands pictured in the lower panels reveals reduced epithelial cell content in Neu/EphA2 −/− tissue samples relative to +/− and +/+ controls. Scale bar=250 mm. (D) Proliferation was assessed by quantification of nuclear staining for PCNA (upper panels, arrows) in tissue sections prepared from mammary glands collected from transgenic animals 8 months after birth. A significant reduction in the percentage of PCNA+ nuclei was observed for MMTV-Neu/EphA2 −/− mammary glands relative to MMTV-Neu/EphA2 +/+ controls (p<0.05; single factor ANOVA). Scale bar=50 mm. No significant change in the percentage of apoptotic nuclei (lower panels, arrows), as assessed by TUNEL assay, was observed in MMTV-Neu/EphA2 −/− mammary glands relative to MMTV-Neu/EphA2 +/+ controls. (E) Proliferation of primary mammary epithelial cells isolated from EphA2 −/− animals, as assessed by nuclear incorporation of BrdU (arrows, upper panels), was also reduced relative to EphA2 +/+ cells (p<0.05; 2-tailed, paired student's T-test). Interestingly, apoptosis (arrows in lower panels show TUNEL+ nuclei) was also significantly impaired in EphA2-deficient primary mammary epithelial cells relative to controls (p<0.05; 2-tailed, paired student's T-test), a phenotype that may be masked by the host microenvironment in mammary epithelium in situ. (F) Hematoxylin and eosin-stained sections from MMTV-Neu tumors collected from transgenic female animals 1 year after birth demonstrate increase cystic degeneration lumen formation in EphA2 −/− tumors relative to +/− and +/+ controls. Scale bar=250 mm. (G) A significant reduction in the percentage of PCNA+ nuclei (arrowheads) was observed for MMTV-Neu/EphA2 −/− tumors compared to MMTV-Neu/EphA2 +/− and +/+ controls (p<0.05; single factor ANOVA). Scale bar=50 mm. (H) Microvascular density, as assessed by immunohistochemical staining for the endothelial specific marker CD31, was significantly reduced in MMTV-Neu/EphA2 −/− tumors relative to +/− and +/+ controls (p<0.05; single factor ANOVA). Arrows indicate CD31+ vessels. Scale bar=100 mm.

FIG. 2. Loss of EphA2 expression impairs tumor formation and invasiveness in MMTV-Neu tumor cells. (A) EphA2 expression was significantly diminished in MMTV-Neu tumor cells transduced with retroviruses expressing EphA2 siRNA sequences, but not in control tumor cells transduced with retroviruses harboring irrelevant control sequences. Diminished expression of EphA2 was accompanied by a reduction in levels of phosphorylated Erk. (B) Cells were plated on growth-factor reduced Matrigel to generate three-dimensional spheroid cultures. After eight days in culture, parental and control siRNA tumor cells formed large, irregularly shaped clusters with invasive protrusions (arrowheads indicate protrusions). By contrast, tumor cells expressing EphA2 siRNA sequences formed smaller clusters that maintained a rounded morphology with few protrusions, indicative of reduced invasiveness. Scale bar=200 mm upper panels, 50 mm lower panels. We observed a significant decrease in colony size, as determined by calculating the average pixel area occupied by individual colonies, for cells in which EphA2 expression was diminished relative to control cells (p<0.05; single factor ANOVA). (C) Three-dimensional cultures were stained with TO-PRO-3 iodide nuclear stain (blue) and anti-E-cadherin (green) and imaged by confocal microscopy. While parental and control siRNA-expressing Neu tumor cells formed multi-acinar structures with invasive protrusions (arrowheads indicate protrusions), tumor cells expressing EphA2 siRNA sequences formed more round, uniform acinar structures composed of a single layer of epithelial cells surrounding a central lumen (arrows indicate lumen). Scale bar=20 mm. (D) Upon orthotopic transplantation into cleared fat pads of FVB recipient female mice, tumor cells expressing control siRNA sequences produced tumors of comparable volume to tumors generated by transplantation of parental MMTV-Neu tumor cells at 5 weeks. Tumor cells expressing EphA2 siRNA sequences, however, either failed to form tumors or formed very small, non-palpable tumors in a small fraction of animals (p<0.05; single factor ANOVA). Data are represented as mean±SEM.

FIG. 3. Elevated EphA2 expression in MCF10A cells overexpressing ErbB2/HER2 enhances growth and invasiveness in vitro. (A) Parental MCF10A human breast cells and MCF10A overexpressing human ErbB2/HER2 were transduced with adenoviruses expressing EphA2 (Ad-EphA2) or control b-galactosidase (Ad-bgal) and plated on growth-factor reduced Matrigel to generate three-dimensional spheroid cultures. After 10 days in culture, parental MCF10A cells and cells expressing Ad-bgal formed small round acinar structures, while MCF10A.HER2 cells formed larger colonies with irregular, invasive protrusions. Expression of Ad-EphA2 in MCF10A cells resulted in larger, irregular colonies, an effect that was amplified in MCF10A.HER2 cells (p<0.05; single factor ANOVA; arrows indicate invasive protrusions). Scale bar=25 mm. (B) Three-dimensional cultures were stained with TO-PRO-3 iodide nuclear stain (red) and anti-Ki67 (green) and imaged by confocal microscopy. Confocal analysis revealed that parental and Ad-bgal transduced MCF10A formed uniform acinar structures composed of a single layer of epithelial cells surrounding a central lumen, while MCF10A.HER2 cells formed multi-acinar structures with invasive protrusions (arrows indicate protrusions) and a poorly-defined lumen containing several cells. MCF10A cells transduced with Ad-EphA2 also formed multi-acinar structures with a poorly-defined lumen containing cells. Invasion and lumen-filling were enhanced in MCF10A.HER2 overexpressing EphA2. Scale bar=20 mm. Quantification of the percentage of nuclei positive for the proliferation marker Ki67 (arrowheads indicate Ki67+ nuclei) demonstrated that EphA2 overexpression significantly enhanced proliferation within acinar structures formed by MCF10A and MCF10A.HER2 cells (p<0.05; single factor ANOVA). (C) Expression of adenoviral gene products and overexpression of ErbB2/HER2 in MCF10A/HER2 cells was confirmed by immunoblot, and uniform loading was verified by immunoblot for actin.

FIG. 4. EphA2 is required for Ras/Erk activation and proliferation in the context Neu/ErbB2-mediated neoplasia. (A) Proliferation of primary mammary tumor cells (PMTCs) isolated from EphA2 −/− animals, as assessed by nuclear incorporation of BrdU (arrows), was reduced relative to EphA2 +/+ cells (p<0.05; 2-tailed, paired student's T-test). Scale bar=20 mm. (B) Ras activity in unstimulated cells, as measured by immunoprecipitation of GTP-bound Ras in tumor cell lysates by GST-Raf Ras-binding domain, was reduced in EphA2-deficient primary tumor cells relative to wild-type cells, as was Erk phosphorylation. Uniform loading was confirmed by immunoblotting for total Ras, total Erk, and actin. EphA2-deficiency and uniform expression of Neu/ErbB2 was confirmed by immunoprecipitation and immunoblotting for EphA2 and ErbB2 in tumor cell lysates. EphA2 was phosphorylated in unstimulated wild-type tumor cells, and no changes in ErbB2 phosphorylation were detected in wild-type versus EphA2-deficient primary tumor cells. (C) Diminished Ras and Erk activity were confirmed in whole tumor extracts isolated from three independent wild-type or EphA2-deficient tumors. (D) For rescue experiments EphA2-deficient MMTV-Neu primary tumor cells were transduced with adenoviruses expressing Erk-1 or control b-galactosidase (bgal) 48 hours prior to BrdU incorporation assay. Overexpression of Erk-1 in EphA2-deficient PMTCs significantly elevated serum-induced proliferation relative to PMTCs expressing control bgal (p<0.05 −/− Ad-bgal versus +/+ or −/− Ad-Erk-1; single factor ANOVA). Expression of adenoviral transgenes was confirmed by immunoblot.

FIG. 5. EphA2 is required for RhoA activation and tumor cell migration in the context of Neu/ErbB2-mediated malignancy. (A) EphA2-deficient PMTCs displayed significantly reduced migration in response to growth media supplemented with 10% serum compared to wild-type PMTCs in transwell migration assays (p<0.05; 2-tailed, paired student's T-test). (B) RhoA activity, as measured by immunoprecipitation of GTP-bound RhoA in tumor cell lysates and in whole tumor extracts by GST-Rhotekin Rho-binding domain, was reduced in EphA2-deficient PMTCs and intact tumors relative to wild-type cells/tumors. Interestingly, we also observed a decrease in total RhoA protein levels in EphA2-deficient MMTV-Neu tumor cells and in whole tumor extracts relative to wild-type controls. No change in GTP-bound, activated Rac or total Rac protein levels in tumor cell lysates from EphA2-deficient and wild-type PMTCs was observed. (C) For rescue experiments, EphA2-deficient MMTV-Neu primary tumor cells were transduced with adenoviruses expressing constitutively active RhoA (Q63L) or control b-galactosidase (bgal) 48 hours prior to migration assay. Expression of constitutively active RhoA restored serum-induced migration of EphA2-deficient tumor cells to levels comparable to those observed in tumor cells derived from wild-type animals, while control b-gal had no effect (p<0.05 −/− Ad-bgal versus +/+ or −/− Ad-Rho; single factor ANOVA). Expression of adenoviral transgenes was confirmed by immunoblot and Rho activity assays.

FIG. 6. EphA2 physically and functionally interacts with ErbB2. (A) Endogenous ErbB2 and EphA2 were co-immunoprecipitated with anti-EphA2 or anti-ErbB2 antibodies, respectively, in wild-type MMTV-Neu tumor cells that were untreated or treated with the chemical crosslinker DSSTP. The interaction detected was specific, as EphA2 and ErbB2 were not immunoprecipitated by control IgG. Uniform input was validated by probing lysates for expression of EphA2 and ErbB2. (B) COS7 cells were transfected with plasmids for expression of EphA2 or ErbB2. EphA2 was immunoprecipitated from cell lysates and products were analyzed for EphA2 and ErbB2. Co-expression of EphA2 and ErbB2 was sufficient to permit co-immunoprecipitation. Uniform transfection efficiency was confirmed by immunoblotting for EphA2 and ErbB2 expression within input lysate. Co-expression of ErbB2 and EphA2 was sufficient to induce phosphorylation of EphA2 in COS7 cells in the absence of stimulation and above basal levels of phosphorylation induced by overexpression of EphA2 alone. (C) Interaction between EphA2 and human ErbB2 (HER2) in MCF10A cells overexpressing HER2 was observed, as EphA2 and HER2 were co-immunoprecipitated with anti-EphA2 antibodies in HER2 overexpressing cells, but not in parental MCF10A. Elevated EphA2 phosphorylation was observed in MCF10A cells overexpressing HER2 relative to parental MCF10A cells, and treatment with the ErbB2 kinase inhibitor AG825 reduced EphA2 phosphorylation as well as ErbB2 phosphorylation in MCF10A cells overexpressing HER2.

FIG. 7. EphA2-deficiency does not affect tumorigenesis, microvascular density, or growth regulatory signaling pathways in MMTV-PyV-mT tumors. (A) No significant differences in tumor volume or the number of lung metastases were observed in MMTV-PyV-mT/EphA2 −/− female animals relative to wild-type or heterozygous controls analyzed 100 days after birth. Data are represented as mean±SEM. (B) Loss of EphA2 protein expression was confirmed in EphA2-deficient PyV-mT tumors by immunohistochemical staining. Scale bar=50 mm. (C) No change in microvascular density, as assessed by immunofluorescent staining for the endothelial marker von Willebrand factor (vWF; arrows indicate vWF+ blood vessels) was detected. Scale bar=100 mm. (D) No change in levels of GTP-bound active Ras or phosphorylated Erk in EphA2-deficient MMTV-PyV-mT whole tumor extracts relative to wild-type controls was observed, nor was any change in expression levels of RhoA observed. Uniform loading was confirmed by immunoblotting for total Ras, total Erk, and tubulin. (E) Expression and phosphorylation of EphA2 in normal mammary tissue isolated from FVB female mice relative to tumor tissue isolated from MMTV-Neu and MMTV-PyV-mT female mice was assessed. EphA2 overexpression and elevated phosphorylation in both tumor types relative to normal tissue was observed, with the highest levels observed in MMTV-Neu tumors. Overexpression of ErbB2 and ephrin-A1 in both tumor types was observed, with comparable ephrin-A1 expression in MMTV-PyV-mT and MMTV-Neu tumors and higher ErbB2 levels in MMTV-Neu tumors. Uniform loading was confirmed by immunoblot for actin. (F) EphA2 overexpression specifically in epithelium was confirmed by comparing EphA2 levels in primary mammary epithelial cell (PMEC) lysates versus primary mammary tumor cells (PMTC) derived from MMTV-Neu and MMTV-PyV-mT mice.

FIG. 8. Treatment with an anti-EphA2 antibody inhibits tumor growth in MMTV-Neu but not MMTV-PyV-mT tumors. (A) Treatment with anti-murine EphA2 antibody diminishes EphA2 expression in tumor cells derived from MMTV-Neu and MMTV-PyV-mT mice. Tumor cells were treated with control IgG (10 mg/ml) or increasing concentrations of anti-EphA2 antibody for 48 hours. Expression of EphA2 was assessed in tumor cell lysates by immunoblot, and uniform loading confirmed by immunoblot for actin. Blots were stripped and re-probed with anti-EphA4 antibodies as a control for antibody specificity. (B) Cells derived from wild-type MMTV-Neu mice were orthotopically transplanted into the cleared fat pads of female FVB recipient mice. Two weeks following transplantation, mice were injected intraperitoneally with anti-EphA2 antibody or control IgG (10 mg/kg) twice weekly for three weeks. Tumors were harvested 5 weeks post-transplantation for analysis. A significant reduction in tumor volume in animals treated with the anti-EphA2 antibody relative to control IgG-treated mice was observed (p<0.05; single factor ANOVA). Data are represented as mean±SEM. (C) Tumor cell proliferation, as assessed by nuclear PCNA expression, was also significantly impaired in anti-EphA2-treated animals relative to controls (p<0.05; single factor ANOVA; black arrowheads indicate PCNA+ nuclei). Scale bar=50 mm. (D) EphA2 expression was significantly diminished in anti-EphA2-treated tumors relative to IgG controls, as assessed by immunohistochemistry (upper panels) and immunoblot (lower panels). Blots were stripped and re-probed for actin expression to verify uniform loading. Scale bar=50 mm. (E) Significantly reduced (p<0.05; single factor ANOVA) microvascular density in tumors isolated from anti-EphA2-treated mice relative to controls based on quantification of vWF fluorescence was observed (white arrowheads indicate vWF+blood vessels). Scale bar=100 mm. (E) Cells derived from MMTV-PyV-mT mice were orthotopically transplanted in the cleared fat pad of FVB female recipient mice and were treated with anti-EphA2 antibody or control IgG as described above. No change in tumor volume between animals treated with anti-EphA2 antibody relative to control IgG-treated mice was observed.

FIG. 9. EphA2-deficiency impairs mammary epithelial penetration of the surrounding fat pad in a subset of MMTV-Neu animals. (A) Whole-mount hematoxylin staining of number 4 inguinal mammary glands collected from EphA2 +/+ and −/− MMTV-Neu female transgenic animals 8 months after birth reveals failure of the mammary epithelium to fully penetrate the mammary fat pad (dashed line shows extent of penetration in the far right panel) past the inguinal lymph node (*), a phenotype observed in approximately 30% of −/− animals. The left and middle panels show whole-mount preparations from +/+ and an independent −/− mammary gland, respectively, for comparison. (B) Immunohistochemical staining for EphA2 was used to confirm loss of EphA2 protein expression in mammary epithelium (arrowheads, upper panels) and mammary blood vessels (arrows, lower panels) in EphA2-deficient tissue samples relative to wild-type controls. Scale bar=50 mm. (C) Immunohistochemical staining for ErbB2 revealed no apparent differences in expression or localization of ErbB2 between EphA2 +/+, +/−, or −/− MMTV-Neu tumors. Scale bar=50 mm.

FIG. 10. Vascular defects observed in MMTV-Neu/EphA2-deficient tumors due in part to loss of EphA2 expression in host endothelium. (A) Tumor cells derived from MMTV-Neu animals were orthotopically transplanted into cleared mammary fat pads wild-type or EphA2-deficient FVB host animals. Relative to wild-type controls, a significant decrease in tumor volume in tumors collected from EphA2-deficient host animals 5 weeks after transplantation was observed (p<0.05; single factor ANOVA). (B) Consistent with previous studies, significantly reduced (p<0.05; ANOVA) microvascular density in tumors isolated from EphA2-deficient hosts versus wild-type controls based on quantification of vWF immunofluorescence was observed (arrowheads indicate vWF+blood vessels). Scale bar=100 mm. (C) To determine if the defects observed in vascular recruitment were due to loss of EphA2 expression in host endothelium, tumor cell-endothelial cell co-culture migration assays were performed (see diagram). Wild-type MMTV-Neu tumor cells labeled with a green fluorescent marker were seeded on the lower surface of a Matrigel-coated transwell. Endothelial cells derived from wild-type or EphA2-deficient animals were labeled with a red fluorescent dye and added to the upper chamber of the transwell and recruitment of endothelial cells to the lower surface by tumor-derived signals was measured. After 5 hours, significantly fewer (p<0.05; 2-tailed, paired student's T-test) EphA2-deficient endothelial cells on the lower surface of the transwell than control wild-type endothelial cells were observed (arrows indicate endothelial cells that migrated to the lower surface of the transwell).

FIG. 11. EphA2-deficiency reduces Erk phosphorylation primary mammary epithelial cells (PMEC) derived from MMTV-Neu mice and phospho-Erk in mammary epithelium. (A) Consistent with the observations in primary tumor cells, PMEC isolated from EphA2-deficient MMTV-Neu animals displayed lower basal levels of phospho-Erk than control PMEC derived from wild-type animals in the absence of changes in the expression levels of ephrin-A1 ligand. EphA2-deficiency was confirmed by immunoprecipitation of EphA2 from PMEC lysates. (B) Consistent with these observations, lower expression of p-Erk in tissue sections prepared from EphA2-deficient versus wild-type MMTV-Neu mammary glands was observed. Scale bar=50 mm.

FIG. 12. Treatment with Anti-EphA2 antibody downregulates EphA2 expression in MMTV-Neu and MMTV-PyV-mT tumor cells but does not affect expression of ErbB2 in MMTV-Neu tumors. (A) Immunohistochemical staining for ErbB2 revealed no apparent differences in expression or localization in MMTV-Neu tumors harvested from animals treated with control IgG versus anti-EphA2 antibodies. Staining specificity for ErbB2 was confirmed by probing adjacent sections with control rabbit IgG. Scale bar=50 mm. (B) Treatment with anti-murine EphA2 antibody had no impact on microvascular density in MMTV-PyV-mT tumors relative to controls based on vWF fluorescence (arrowheads indicate vWF+blood vessels). Scale bar=100 mm. (B) Treatment with anti-murine EphA2 antibody diminishes EphA2 expression in MMTV-PyV-mT tumors relative to control tumor-bearing animals treated with IgG. Scale bar=50 mm

### 7. DETAILED DESCRIPTION

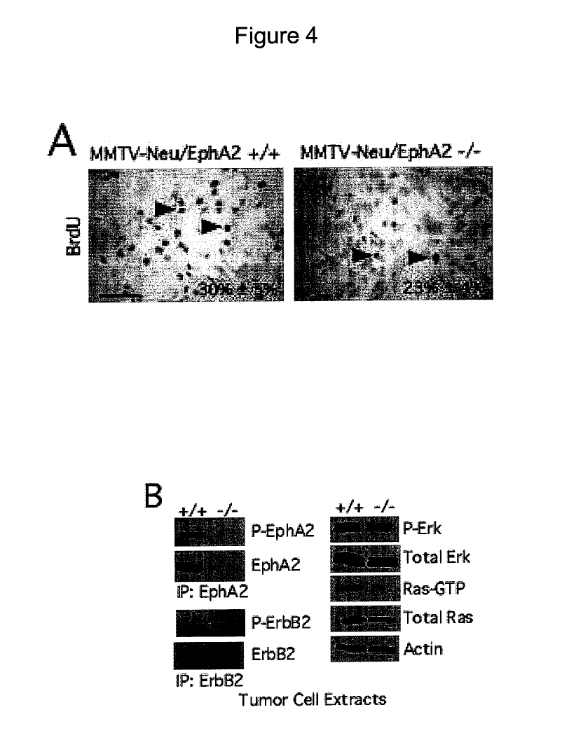

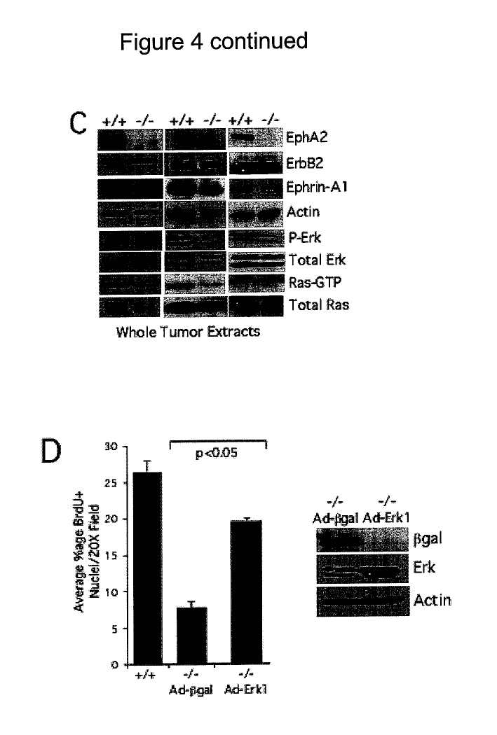

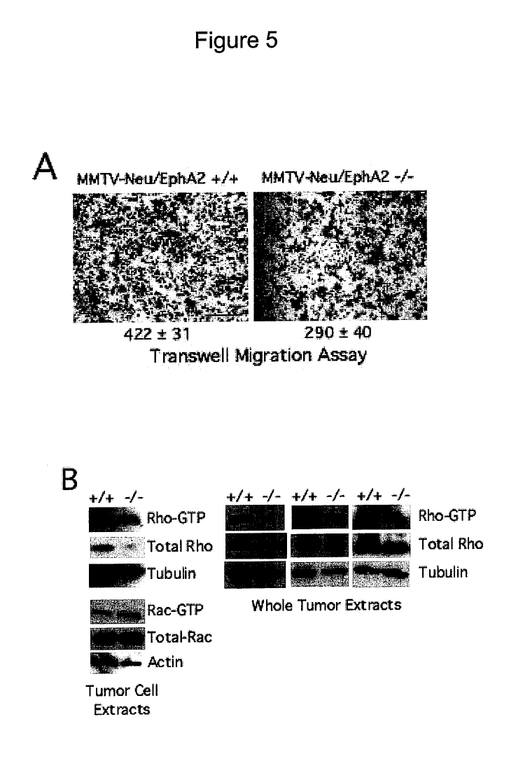

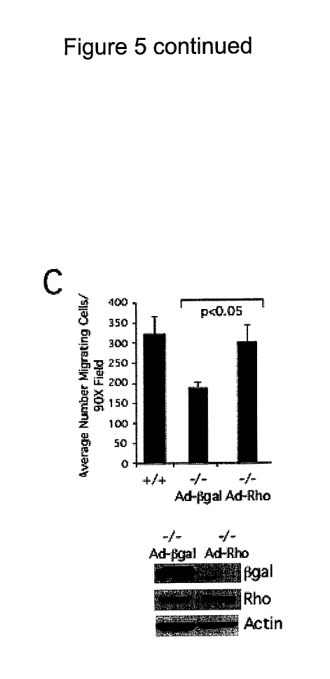

A large body of work indicates that tumorigenesis is a multi-step process and different oncogenes often cooperate to promote different steps of tumor progression [Reviewed in (Hahn and Weinberg, 2002; Hanahan and Weinberg, 2000; Vogelstein and Kinzler, 2004)]. Applicants have demonstrated a physical interaction between EphA2 and ErbB2 at the tumor cell surface, inducing phosphorylation of the EphA2 receptor in the absence of ligand stimulation. This interaction between ErbB2 and EphA2 amplifies Ras/Erk signaling and Rho GTPase activation (FIGS. 4 and 5), likely contributing to the increased proliferation and motility of EphA2-expressing tumor cells. This observation holds repercussions regarding how ErbB2-expressing breast cancers are treated, especially those that are refractory to anti-ErbB2 therapies. These results suggest that anti-EphA2 therapy may be effective against ErbB2-expressing tumors, alone or in combination with methods targeting ErbB2.

Although EphA2 is overexpressed in a wide variety of tumors, including breast adenocarcinomas [Reviewed in (Brantley-Sieders et al., 2004a; Brantley-Sieders and Chen, 2004; Ireton and Chen, 2005)], the present invention provides that overexpression in and of itself does not necessarily indicate an active role in tumorigenesis. Significant levels of EphA2 overexpression were documented in tumors arising in both MMTV-Neu and MMTV-PyV-mT models of mammary carcinogenesis in this study. However, while deletion of EphA2 significantly impairs tumor initiation and progression in MMTV-Neu animals, there was no effect of EphA2-deficiency on tumor progression in the MMTV-PyV-mT model, which expresses only moderate levels of ErbB2. Thus, the functional consequences of EphA2 overexpression depend upon the context of co-expressed oncogenes. Therefore, effective therapeutic targeting of EphA2 requires an understanding of how EphA2 cooperates with and functionally influences co-existing oncogenic signaling networks within specific tumor types. For example, while downregulation of EphA2 protein levels showed efficacy against human ovarian tumor xenografts (Landen et al., 2006), an independent, similarly-designed antibody reagent had no effect on CT26 human colon cancer xenografts or human mammary adenocarcinoma xenografts (Kiewlich et al., 2006). Interestingly, like MMTV-PyV-mT tumor cells, CT26 cells do not overexpress ErbB2/HER2 (Penichet et al., 1999), suggesting that EphA2 overexpression enhances malignant transformation and progression particularly in the context of ErbB2 overexpression, and is therefore an appropriate target in such tumors.

While EphA2 overexpression has been reported in a variety of human epithelial cancers, including more than 80% of breast cancer clinical samples (Ogawa et al., 2000; Pan, 2005; Zelinski et al., 2001), HER2 overexpression is observed in only approximately 30% of human breast cancers (Ursini-Siegel et al., 2007). Moreover, no correlation was reported between EphA2 and HER2 expression in a recent screen of 134 human breast cancer specimens (Pan, 2005). The present invention provides that EphA2 interacts with ErbB2. Other EGFR family members, including epidermal growth factor receptor (EGFR/ErbB1) and an EGFR variant (EGFRvIII) that is constitutively active deletion mutant implicated in carcinogenesis, have also been shown to physically and functionally interact EphA2 (Larsen et al., 2007).

Moreover, EGFR activation elevates expression of EphA2 (Larsen et al., 2007; Ramnarain et al., 2006). Overexpression of EGFRvIII enhances tumorigenesis in human tumor xenografts (Tang et al., 2000), and mammary epithelial specific overexpression of EGFR in transgenic animals induces neoplasia (Brandt et al., 2000). In addition, overexpression of EGFR and EGFRvIII has been reported in a broader subset of human breast cancers, with as many as 48% of cases analyzed reported to be positive for EGFR expression (Ge et al., 2002; Klijn et al., 1992; Klijn et al., 1994; Rae et al., 2004; Tsutsui et al., 2003; Wikstrand et al., 1995). Thus, EphA2 may act in concert with the EGFR family of receptor tyrosine kinases in general, and not exclusively with ErbB2, to enhance proliferation and malignant progression. As ErbB2/HER2 is an orphan receptor, heterodimerization with other EGFR family members induces ErbB2 activation [Reviewed in (Ursini-Siegel et al., 2007)]. Interactions between EGFR and ErbB2 affect breast tumor progression, as inhibition of EGFR kinase activity reduces HER2/Neu phosphorylation in breast cancer cell lines in vitro, enhances the anti-tumor effects of herceptin in xenografts, and suppresses tumorigenesis in MMTV-Neu/MMTV-TGFa bigenic mice (Moulder et al., 2001), (Lenferink et al., 2000). Thus, functional interaction between EphA2 and EGFR as well as ErbB2 may be required for breast tumor growth and progression.

Accordingly, the present invention provides that the role of EphA2 receptor tyrosine kinase in cancer is context dependent, as EphA2 deficiency impairs tumor progression in MMTV-Neu, but not MMTV-PyV-mT transgenic models of mammary epithelial adenocarcinoma. The present invention provides evidence that EphA2 physically and functionally interacts with ErbB2 to amplify Ras/MAPK and RhoA signaling in tumor cells. Ras/MAPK contributes to cell proliferation, while activated Rho GTPase is required for tumor cell motility. Together, these results indicate that EphA2 cooperates with ErbB2/Neu to promote tumor progression and that EphA2 is a novel target for tumors that are dependent upon ErbB-receptor signaling.

### 7.1 Embodiments of the Invention

Embodiments of the invention are provided in the following numbered embodiments:

1. A method of reducing proliferation of hyperproliferative cells, said method comprising:
a) identifying a population of hyperproliferative cells that express both EphA2 and ErbB2; and
b) administering an agent that targets EphA2.
   2. A method of reducing proliferation of hyperproliferative cells that express both EphA2 and ErbB2 comprising administering an agent that targets EphA2
   3. A method of reducing proliferation of hyperproliferative cells that express both EphA2 and ErbB2 comprising administering an agent that inhibits, blocks, or interferes with the interaction between EphA2 and ErbB2.
   4. The method of any of embodiments 1-3, wherein said hyperproliferative cells are cancer cells.
   5. The method of embodiment 4, wherein said cancer is of the skin, lung, colon, breast, prostate, bladder or pancreas, a renal cell carcinoma, a melanoma, a leukemia, or a lymphoma.
   6. The method of any of embodiments 1-3, wherein said hyperproliferative cell disease is a non-cancer hyperproliferative cell disease.
   7. The method of embodiment 6, wherein said non-cancer hyperproliferative cell disease is asthma, chronic obstructive pulmonary disease (COPD), psoriasis, lung fibrosis, bronchial hyper responsiveness, seborrheic dermatitis, and cystic fibrosis, inflammatory bowel disease, smooth muscle restenosis, endothelial restenosis, hyperproliferative vascular disease, Behcet's Syndrome, atherosclerosis, or macular degeneration.
   8. The method of any of embodiments 1-7, further comprising administering an agent that targets ErbB2.
   9. The method of any of embodiments 1-8, wherein said cells overexpress EphA2.
   10. The method of any of embodiments 1-8, wherein said cells overexpress ErbB2.
   11. The method of any of embodiment 1-10, wherein said cells overexpress both EphA2 and ErbB2.
   12. The method of any of embodiments 1-11, wherein said EphA2 targeting agent is agonistic.
   13. The method of any of embodiments 1-11, wherein said EphA2 targeting agent is antagonistic.
   14. The method of any of embodiments 1-13, wherein said EphA2 or ErbB2 targeting agent is an antibody.
   15. The method of any of embodiments 1-13, wherein said EphA2 or ErbB2 targeting agent is a small molecule.
   16. The method of any of embodiments 1-13, wherein said EphA2 or ErbB2 targeting agent is a peptide.
   17. The method of embodiments 1-13, wherein said EphA2 or ErbB2 targeting agent is an siRNA.
   18. The method of embodiments 1-13, wherein said EphA2 or ErbB2 targeting agent is an antibody-drug conjugate (ADC).
   19. The method of any of embodiments 1-18, wherein any of said EphA2 or ErbB2 targeting agents inhibits, blocks, or interferes with the interaction between EphA2 and ErbB2.
   20. A method of treating a cancer patient, said method comprising:
(a) determining the expression level, presence, or amount of EphA2 and ErbB2 in said patient's cancer cells;
(b) administering an anti-EphA2 and/or an anti-ErbB2 targeting agent if it is determined that said patient's cancer cells express both EphA2 and ErbB2.

### 7.2 Agents that Target EphA2 or ErbB2

Agents that target EphA2 or ErbB2 and alter their expression and/or activity can be assessed in comparison to EphA2 or ErbB2 expression and/or activity prior to treatment. Typically this is assessed in a laboratory setting using appropriate cell lines that are known in the art. It should be understood that the method of the invention is not limited by the way in which, or the extent to which, EphA2 or ErbB2 expression and/or activity is inhibited in the target cells.

Methods for altering Epha2 or ErbB2 expression and/or activity include, but are not limited to, those that alter EphA2 or ErbB2 expression or activity, e.g., agents that antagonize EphA2 or ErbB2, agents that agonize EphA2, agents that lead to increased phosphorylation of EphA2, agents that lead to degradation of EphA2 (specifically, those that bind to epitopes of EphA2 exposed on cancer cells and those agonize EphA2), those that can be used as vaccines, those that act on the mRNA transcript produced by the gene encoding EphA2 or ErbB2, those that interfere with the translation of the mRNA transcript into the protein, and those that directly impair the activity of the translated protein.

Transcription of a gene can be impeded by delivering to the cell an antisense DNA or RNA molecule, a double stranded RNA molecule. Another way the activity of an enzyme can be inhibited is by interfering with the mRNA transcription product of the gene. For example, a ribozyme (or a DNA vector operably encoding a ribozyme) can be delivered to the cell to cleave the target mRNA. Antisense nucleic acids and double stranded RNAs may also be used to interfere with translation.

Peptides, polypeptides (including antibodies, antibody fragments, fusion proteins), ligands, ligand mimics, peptidomimetic compounds and other small molecules are examples of those that can be used to directly compromise the activity of the translated protein. Alternatively, a proteinaceous intracellular agent that alters the expression and/or activity of Epha2 or ErbB2 can be delivered as a nucleic acid, for example as RNA, DNA, or analogs or combinations thereof, using conventional methods, wherein the therapeutic polypeptide is encoded by the nucleic acid and operably linked to regulatory elements such that it is expressed in the target mammalian cell.

Other therapeutic or prophylactic agents for use in altering EphA2 or ErbB2 expression and/or activity include, but are not limited to, small molecules, peptides, antisense oligonucleotides, avimers, aptamers, substrate mimics (e.g., non-hydrolyzable or substrate trapping inhibitors) and agents that can be used as vaccines to generate antibodies against Epha2 or ErbB2. Treatment agents can include antagonists that resemble substrate or that interfere with the binding of Epha2 or ErbB2 to its substrate, particularly those that interfere with EphA2-ErbB2 interactions. In one embodiment, an agent that alters Epha2 or ErbB2 activity is an agent that prevents ErbB2 from binding phosphorylated EphA2. In another embodiment, an agent that alters Epha2 or ErbB2 activity is an agent that prevents ErbB2 from binding EphA2, regardless whether EphA2 is phosphorylated. Non-limiting examples of agents that can be used to alter EphA2 or ErbB2 expression and/or activity include small molecules, Ephrin peptides (particularly Ephrin A1 that binds EphA2) or Ephrin peptide fusion proteins, EphA2 binding antibodies and fragments thereof, antisense oligonucleotides, RNA interference (RNAi) molecules, avimers, and aptamers.

### 7.2.1 Antibodies

Antibodies are immunological proteins that bind a specific antigen. In most mammals, including humans and mice, antibodies are constructed from paired heavy and light polypeptide chains. Each chain is made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of natural proteins to elicit important biochemical events.

The Fc region of an antibody interacts with a number of ligands including Fc receptors and other ligands, imparting an array of important functional capabilities referred to as effector functions. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CID64), including isoforms FcγRIA, FcγRIB, and FcγRIC; FcγRII (CD32), including isoforms FcγRIIA, FcγRIIB, and FcγRIIC; and FcγRIII (CID16), including isoforms FcγRIIIA and FcγRIIB (Jefferis et al., 2002, Immunol Lett 82:57-65). These receptors typically have an extracellular domain that mediates binding to Fc, a membrane spanning region, and an intracellular domain that may mediate some signaling event within the cell. These different FcγR subtypes are expressed on different cell types (reviewed in Ravetch et al., 1991, Annu Rev Immunol 9:457-492). For example, in humans, FcγRIIIB is found only on neutrophils, whereas FcγRIIIA is found on macrophages, monocytes, natural killer (NK) cells, and a subpopulation of T-cells.

Formation of the Fc/FcγR complex recruits effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). Notably, the primary cells for mediating ADCC, NK cells, express only FcγRIIIA, whereas monocytes express FcγRI, FcγRII and FcγRIII (Ravetch et al., 1991).

Another important Fc ligand is the complement protein C1q. Fc binding to C1q mediates a process called complement dependent cytotoxicity (CDC) (reviewed in Ward et al., 1995, Ther Immunol 2:77-94). C1q is capable of binding six antibodies, although binding to two IgGs is sufficient to activate the complement cascade. C1q forms a complex with the C1r and C1s serine proteases to form the C1 complex of the complement pathway.

Several key features of antibodies including but not limited to, specificity for target, ability to mediate immune effector mechanisms, and long half-life in serum, make antibodies and related immunoglobulin molecules powerful therapeutics. Numerous monoclonal antibodies are currently in development or are being used therapeutically for the treatment of a variety of conditions including cancer. Examples of these include Vitaxin® (MedImmune), a humanized Integrin αvβ3 antibody (e.g., PCT publication WO 2003/075957), Herceptin® (Genentech), a humanized anti-Her2/neu antibody approved to treat breast cancer (e.g., U.S. Pat. No. 5,677,171), CNTO 95 (Centocor), a human Integrin αv antibody (PCT publication WO 02/12501), Rituxan® (IDEC/Genentech/Roche), a chimeric anti-CD20 antibody approved to treat Non-Hodgkin's lymphoma (e.g., U.S. Pat. No. 5,736,137) and Erbitux® (ImClone), a chimeric anti-EGFR antibody (e.g., U.S. Pat. No. 4,943,533).

There are a number of possible mechanisms by which antibodies destroy tumor cells, including anti-proliferation via blockage of needed growth pathways, intracellular signaling leading to apoptosis, enhanced down regulation and/or turnover of receptors, ADCC, CDC, and promotion of an adaptive immune response (Cragg et al., 1999, Curr Opin Immunol 11:541-547; Glennie et al., 2000, Immunol Today 21:403-410). Although naked antibodies may be effective in their desired therapeutic uses, in certain instances, altering the antibody may enhance efficacy. For example, but not limited to, altering the Fc region of the antibody to increase or decrease effector function.

In another embodiment, the antibodies of the invention are variants of antibodies that specifically bind EphA2, their derivatives, analogs and epitope-binding fragments thereof, such as but not limited to, those disclosed herein and in PCT Publication Nos. WO 04/014292, WO 03/094859, U.S. Patent Application Publications 2007/0134254, 2006/0177453, 2006/0039904, 2004/0028685 and U.S. Provisional Patent Application 60/751,964, filed on Dec. 21, 2005 and 60/842,641, filed on Sep. 7, 2006, each of which is incorporated herein by reference in its entirety. In a specific embodiment, the antibodies of the invention are antibodies that specifically bind EphA2 which comprise all or a portion of the variable region (e.g., one or more CDR) from the anti-EphA2 antibodies 3F2, 1C1, 1F12, 1H3, 1D3, 2B12, 5A8 as disclosed in the aforementioned patent applications.

In one embodiment, the antibodies of the invention bind ErbB2. Examples include, but are not limited to, those disclosed in U.S. Pat. Nos. 5,677,171 and 6,458,356 and U.S. Patent Application Publication Nos. 2007/0037228 and 2006/0275305.

The present invention further encompasses the use of antibodies of the invention that have a high binding affinity for at least one Eph receptor or for ErbB2. In a specific embodiment, an antibody of the invention that specifically binds to at least one Eph receptor or ErbB2 has an association rate constant or kon rate ((Ab)+antigen (Ag)kon←Ab-Ag) of at least 10⁵M⁻¹s⁻¹, at least 5×10⁵M⁻¹s⁻¹, at least 10⁶M⁻¹s⁻¹, at least 5×10⁶M⁻¹s⁻¹, at least 107M⁻¹s⁻¹, at least 5×10⁷M⁻¹s⁻¹, or at least 10⁸M⁻¹s⁻¹. In a further specific embodiment, an antibody of the invention that specifically binds to at least one Eph receptor or ErbB2 has an association rate constant or kon rate ((Ab)+antigen (Ag)kon←Ab-Ag) of at least about 105M⁻¹s⁻¹, at least about 5×10⁵M⁻¹s⁻¹, at least about 10⁶M⁻¹s⁻¹, at least about 5×10⁶M⁻¹s⁻¹, at least about 10⁷M⁻¹s⁻¹, at least about 5×10⁷M⁻¹s⁻¹, or at least about 10⁸M⁻¹s⁻¹. In another embodiment, an antibody that specifically binds to at least one Eph receptor or ErbB2 has a kon of at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹. In a further embodiment, an antibody that specifically binds to at least one Eph receptor or ErbB2 has a kon of at least about 2×10⁵M⁻¹s⁻¹, at least about 5×10⁵M⁻¹s⁻¹, at least about 10⁶M⁻¹s⁻¹, at least about 5×10⁶M⁻¹s⁻¹, at least about 10⁷M⁻¹s⁻¹, at least about 5×10⁷M⁻¹s⁻¹, or at least about 10⁸M⁻¹s⁻¹.

In another embodiment, an antibody of the invention that specifically binds to least on Eph receptor or ErbB2 has a k_{off} rate ((Ab)+antigen (Ag)^{k}_{off}←Ab-Ag) of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰⁻¹s⁻¹. In still another embodiment, an antibody of the invention that specifically binds to least on Eph receptor or ErbB2 has a k_{off} rate ((Ab)+antigen (Ag)^{k}_{off}←Ab-Ag) of less than about 10⁻¹s⁻¹, less than about 5×10⁻¹s⁻¹, less than about 10⁻²s⁻¹, less than about 5×10⁻²s⁻¹, less than about 10⁻³s⁻¹, less than about 5×10⁻³s⁻¹, less than about 10⁻⁴s⁻¹, less than about 5×10⁻⁴s⁻¹, less than about 10⁻⁵s⁻¹, less than about 5×10⁻⁵s⁻¹, less than about 10⁻⁶s⁻¹, less than about 5×10⁻⁶s⁻¹, less than about 10⁻⁷s⁻¹, less than about 5×10⁻⁷s⁻¹, less than about 10⁻⁸s⁻¹, less than about 5×10⁻⁸s⁻¹, less than about 10⁻⁹s⁻¹, less than about 5×10⁻⁹s⁻¹, or less than about 10⁻¹⁰⁻¹s⁻¹. In a further embodiment, an antibody that specifically binds to least on Eph receptor or ErbB2 has a k_{off}, of less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹. In another embodiment, an antibody that specifically binds to least on Eph receptor or ErbB2 has a k_{off}, of less than about 5×10⁻⁴s⁻¹, less than about 10⁻⁵s⁻¹, less than about 5×10⁻⁵s⁻¹, less than about 10⁻⁶s⁻¹, less than about 5×10⁻⁶s⁻¹, less than about 10⁻⁷s⁻¹, less than about 5×10⁻⁷s⁻¹, less than about 10⁻⁸s⁻¹, less than about 5×10⁻⁸s⁻¹, less than about 10⁻⁹s⁻¹, less than about 5×10⁻⁹s⁻¹, or less than about 10⁻¹⁰s⁻¹.

In another embodiment, an antibody of the invention that specifically binds to least on Eph receptor or ErbB2 has an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹M⁻¹, at least 10¹¹M⁻¹, at least 5×10¹¹M⁻¹, at least 10¹²M⁻¹, at least 5×10¹²M, at least 10¹³M⁻¹, at least 5×10¹³M⁻¹, at least 10¹⁴M⁻¹, at least 5×10¹⁴M⁻¹, at least 10¹⁵M⁻¹, or at least 5×10¹⁵M⁻¹. In a further embodiment, an antibody of the invention that specifically binds to least on Eph receptor or ErbB2 has an affinity constant or Kₐ (kₒₙ/k_{off}) of at least about 10²M⁻¹, at least about 5×10²M⁻¹, at least about 10³M⁻¹, at least about 5×10³M⁻¹, at least about 10⁴M⁻¹, at least about 5×10⁴M⁻¹, at least about 10⁵M⁻¹, at least about 5×10⁵M⁻¹, at least about 10⁶M⁻¹, at least about 5×10⁶M⁻¹, at least about 10⁷M⁻¹, at least about 5×10⁷M⁻¹, at least about 10⁸M⁻¹, at least about 5×10⁸M⁻¹, at least about 10⁹M⁻¹, at least about 5×10⁹M⁻¹, at least about 10¹⁰M⁻¹, at least about 5×10¹M⁻¹, at least about 10¹¹M⁻¹, at least about 5×10¹¹M⁻¹, at least about 10¹²M⁻¹, at least about 5×10¹²M, at least about 10¹³M⁻¹, at least about 5×10¹³M⁻¹, at least about 10¹⁴M⁻¹, at least about 5×10¹⁴M⁻¹, at least about 10¹⁵M⁻¹, or at least about 5×10¹⁵M⁻¹.

In yet another embodiment, an antibody that specifically binds to least on Eph receptor or ErbB2 has a dissociation constant or K_{d} (k_{off}/kₒₙ) of less than 10⁻²M, less than 5×10⁻²M, less than 10⁻³M, less than 5×10⁻³M, less than 10⁻⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸M, less than 10⁻⁹M, less than 5×10⁻⁹M, less than 10⁻¹⁰M, less than 5×10⁻¹⁰M, less than 10⁻¹¹M, less than 5×10⁻¹¹M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M, or less than 5×10⁻¹⁵M. In a further embodiment, an antibody that specifically binds to least on Eph receptor or ErbB2 has a dissociation constant or K_{d} (k_{off}/kₒₙ) of less than about 10⁻²M, less than about 5×10⁻²M, less than about 10⁻³M, less than about 5×10⁻³M, less than about 10⁻⁴M, less than about 5×10⁻⁴M, less than about 10⁻⁵M, less than about 5×10⁻⁵M, less than about 10⁻⁶M, less than about 5×10⁻⁶M, less than about 10⁻⁷M, less than about 5×10⁻⁷M, less than about 10⁻⁸M, less than about 5×10⁻⁸M, less than about 10⁻⁹M, less than about 5×10⁻⁹M, less than about 10⁻¹° M, less than about 5×10⁻¹⁰M, less than about 10⁻¹¹M, less than about 5×10⁻¹¹M, less than about 10⁻¹²M, less than about 5×10⁻¹²M, less than about 10⁻¹³M, less than about 5×10⁻¹³M, less than about 10⁻¹⁴M, less than about 5×10⁻¹⁴M, less than about 10⁻¹⁵M, or less than about 5×10⁻¹⁵M.

The present invention also provides antibodies that specifically bind to an EphA2 polypeptide, said antibodies comprising a VH CDR having an amino acid sequence of any one of the VH CDRs of the antibodies discussed herein. In particular, the invention provides antibodies that specifically bind to an EphA2 polypeptide, said antibodies comprising (or alternatively, consisting of, or consisting essentially of) one, two, three or more VH CDRs having an amino acid sequence of any of the VH CDRs of the antibodies discussed herein.

The present invention also provides antibodies that specifically bind to an EphA2 polypeptide, said antibodies comprising a VL CDR having an amino acid sequence of any one of the VL CDRs of the antibodies discussed herein. In particular, the invention provides antibodies that specifically bind to an EphA2 polypeptide, said antibodies comprising (or alternatively, consisting of, or consisting essentially of) one, two, three or more VL CDRs having an amino acid sequence of any of the VL CDRs of the antibodies discussed herein.

The present invention provides antibodies that specifically bind to an EphA2 polypeptide or that specifically bind to an ErbB2 polypeptide, said antibodies comprising a VH domain disclosed herein combined with a VL domain disclosed herein, or other known VL domains. The present invention also provides antibodies that specifically bind to an EphA2 polypeptide or that specifically bind to an ErbB2 polypeptide, said antibodies comprising a VL domain disclosed herein combined with a VH domain disclosed herein, or other known VH domains.

The present invention contemplates the use of, and provides antibodies that specifically bind to an EphA2 polypeptide or that specifically bind to an ErbB2 polypeptide, said antibodies comprising one or more VH CDRs and one or more VL CDRs of the antibodies discussed herein. In particular, the invention provides an antibody that specifically binds to an EphA2 polypeptide or that specifically bind to an ErbB2 polypeptide, said antibody comprising (or alternatively, consisting of, or consisting essentially of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VII CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs of the antibodies discussed herein.

Peptides, polypeptides or proteins comprising one or more variable or hypervariable regions have utility, e.g., in the production of anti-idiotypic antibodies which in turn may be used to prevent, treat, and/or ameliorate one or more symptoms associated with a disease or disorder (e.g., cancer, a hyper- or hypo-proliferative disorder). The anti-idiotypic antibodies produced can also be utilized in immunoassays, such as, e.g., ELISAs, for the detection of antibodies which comprise a variable or hypervariable region contained in the peptide, polypeptide or protein used in the production of the anti-idiotypic antibodies.

The present invention provides for antibodies that specifically bind to an EphA2 polypeptide which have an extended half-life in vivo. In particular, the present invention provides antibodies that specifically bind to an EphA2 polypeptide or that specifically bind to an ErbB2 polypeptide which have a half-life in a subject, preferably a mammal and most preferably a human, of greater than 3 days, greater than 7 days, greater than 10 days, greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (for example, a Fc or hinge-Fc domain fragment). See, e.g., International Publication No. WO 98/23289; International Publication No. WO 97/34631; International Publication No. WO 02/060919; and U.S. Pat. No. 6,277,375, each of which is incorporated herein by reference in its entirety.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622, all of which are incorporated herein by reference.

In order for certain antibodies of the invention to perform as required, a key aspect of the antibodies to be conjugated to the toxin of choice is that the antibody, once bound to the cell surface target (e.g. EphA2), is internalized by the cell. Once internalized, the conjugated toxin can be released, or remain bound, to exert its toxic effect on the cell.

The antibody portion of the antibodies of the invention may include, but are not limited to, synthetic antibodies, monoclonal antibodies, oligoclonal antibodies recombinantly produced antibodies, intrabodies, multispecific antibodies, bispecific antibodies, human antibodies, humanized antibodies, chimeric antibodies, synthetic antibodies, single-chain FvFcs (scFvFc), single-chain Fvs (scFv), and anti-idiotypic (anti-Id) antibodies. In particular, antibodies used in the methods of the present invention include immunoglobulin molecules and immunologically active portions of immunoglobulin molecules. The antibodies of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

The antibody portion of the antibodies of the invention may be from any animal origin including birds and mammals (e.g., human, murine, donkey, sheep, rabbit, goat, guinea pig, camel, horse, or chicken). These antibodies can be human or humanized monoclonal antibodies. As used herein, “human” antibodies include antibodies having the amino acid sequence of a human immunoglobulin and include antibodies isolated from human immunoglobulin libraries or from mice that express antibodies from human genes.

Antibodies like all polypeptides have an Isoelectric Point (pI), which is generally defined as the pH at which a polypeptide carries no net charge. It is known in the art that protein solubility is typically lowest when the pH of the solution is equal to the isoelectric point (pI) of the protein. It is possible to optimize solubility by altering the number and location of ionizable residues in the antibody to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of an antibody that result in changes of the pI of said antibody may improve solubility and/or the stability of the antibody. One skilled in the art would understand which amino acid substitutions would be most appropriate for a particular antibody to achieve a desired pI. The pI of a protein may be determined by a variety of methods including but not limited to, isoelectric focusing and various computer algorithms (see for example Bjellqvist et al., 1993, Electrophoresis 14:1023). In one embodiment, the pI of the antibodies of the invention is between is higher then about 6.5, about 7.0, about 7.5, about 8.0, about 8.5, or about 9.0. In another embodiment, the pI of the antibodies of the invention is between is higher then 6.5, 7.0, 7.5, 8.0, 8.5, or 9.0. In one embodiment, substitutions resulting in alterations in the pI of the antibody of the invention will not significantly diminish its binding affinity for an Eph receptor or ErbB2. As used herein the pI value is defined as the pI of the predominant charge form. The pI of a protein may be determined by a variety of methods including but not limited to, isoelectric focusing and various computer algorithms (see, e.g., Bjellqvist et al., 1993, Electrophoresis 14:1023).

The Tm of the Fab domain of an antibody, can be a good indicator of the thermal stability of an antibody and may further provide an indication of the shelf-life. A lower Tm indicates more aggregation/less stability, whereas a higher Tm indicates less aggregation/more stability. Thus, antibodies having higher Tm are frequently used. In one embodiment, the Fab domain of an antibody has a Tm value higher than at least 50° C., 55° C., 60° C., 65° C., 70° C., 75° C., 80° C., 85° C., 90° C., 95° C., 100° C., 105° C., 110° C., 115° C. or 120° C. In another embodiment, the Fab domain of an antibody has a Tm value higher than at least about 50° C., about 55° C., about 60° C., about 65° C., about 70° C., about 75° C., about 80° C., about 85° C., about 90° C., about 95° C., about 100° C., about 105° C., about 110° C., about 115° C. or about 120° C. Thermal melting temperatures I of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154).

The antibodies of the invention may be monospecific, bispecific, trispecific or have greater multispecificity. Multispecific antibodies may specifically bind to different epitopes of desired target molecule or may specifically bind to both the target molecule as well as a heterologous epitope, such as a heterologous polypeptide or solid support material. See, e.g., International Publication Nos. WO 94/04690; WO 93/17715; WO 92/08802; WO 91/00360; and WO 92/05793; Tuft, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547; each of which is incorporated herein by reference in their entireties). In one embodiment, one of the binding specificities is for an Eph receptor, the other one is for ErbB2.

Multispecific antibodies have binding specificities for at least two different antigens (for example, but not limited to, EphA2 and ErbB2). While such molecules normally will only bind two antigens (i.e. bispecific antibodies, BsAbs), antibodies with additional specificities such as trispecific antibodies are encompassed by the instant invention. Examples of BsAbs include without limitation those with one arm directed against EphA2 and the other arm directed against any other antigen. Methods for making bispecific antibodies are known in the art. Traditional production of full-length bispecific antibodies is based on the coexpression of two immunoglobulin heavy chain-light chain pairs, where the two chains have different specificities (Millstein et al., 1983, Nature, 305:537-539 which is incorporated herein by reference in its entirety). Because of the random assortment of immunoglobulin heavy and light chains, these hybridomas (quadromas) produce a potential mixture of different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J., 10:3655-3659. A more directed approach is the generation of a Di-diabody, or a tetravalent bispecific antibody. Methods for producing a Di-diabody are known in the art (see e.g., Lu et al., 2003, J Immunol Methods 279:219-32; Marvin et al., 2005, Acta Pharmacolical Sinica 26:649).

According to a different approach, antibody variable domains with the desired binding specificities (antibody-antigen combining sites) are fused to immunoglobulin constant domain sequences. The fusion can be with an immunoglobulin heavy chain constant domain, comprising at least part of the hinge, CH2, and CH3 regions. In one embodiment, the first heavy-chain constant region (CH1) containing the site necessary for light chain binding is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when, the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm (e.g., an Eph receptor), and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 (incorporated herein by reference in its entirety). For further details of generating bispecific antibodies see, for example, Suresh et al., 1986, Methods in Enzymology, 121:210 (incorporated herein by reference in its entirety). According to another approach described in WO96/27011 (incorporated herein by reference in its entirety), a pair of antibody molecules can be engineered to maximize the percentage of heterodimers which are recovered from recombinant cell culture. In one embodiment, the interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

In a specific embodiment, antibodies for use in the methods of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19xCD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the heavy chain (VH) and light chain (VL) variable domains with a flexible linker to create a single chain, bispecific antibody.

In an embodiment of this invention, an antibody or ligand that specifically binds a polypeptide of interest (e.g., an Eph receptor and/or an Ephrin) will comprise a portion of the BiTE molecule. For example, the VH and/or VL (e.g. a scFV) of an antibody that binds a polypeptide of interest (e.g., an Eph receptor and/or ErbB2) can be fused to an anti-CD3 binding portion such as that of the molecule described above, thus creating a BiTE molecule that targets the polypeptide of interest (e.g., an Eph receptor and/or ErbB2). In addition to the heavy and/or light chain variable domains of antibody against a polypeptide of interest (e.g., an Eph receptor and/or ErbB2), other molecules that bind the polypeptide of interest (e.g., an Eph receptor and/or ErbB2) can comprise the BiTE molecule, for example receptors (e.g., an Eph receptor and/or ErbB2). In another embodiment, the BiTE molecule can comprise a molecule that binds to other T cell antigens (other than CD3). For example, ligands and/or antibodies that specifically bind to T-cell antigens like CD2, CD4, CD8, CD11a, TCR, and CD28 are contemplated to be part of this invention. This list is not meant to be exhaustive but only to illustrate that other molecules that can specifically bind to a T cell antigen can be used as part of a BiTE molecule. These molecules can include the VH and/or VL portions of the antibody or natural ligands (for example LFA3 whose natural ligand is CD3).

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). The above references are each incorporated herein by reference in their entireties. Heteroconjugate antibodies may be made using any convenient cross-linking methods. Suitable cross-linking agents are well known in the art, and are disclosed in U.S. Pat. No. 4,676,980, along with a number of cross-linking techniques. Each of the above references is incorporated herein by reference in its entirety.

Antibodies with more than two valencies incorporating at least one hinge modification of the invention are contemplated. For example, trispecific antibodies can be prepared. See, e.g., Tutt et al. J. Immunol. 147: 60 (1991), which is incorporated herein by reference.

The antibodies of the invention encompass single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties).

Other antibodies specifically contemplated are “oligoclonal” antibodies. As used herein, the term “oligoclonal” antibodies” refers to a predetermined mixture of distinct monoclonal antibodies. See, e.g., PCT publication WO 95/20401; U.S. Pat. Nos. 5,789,208 and 6,335,163 which are incorporated by reference herein. Oligoclonal antibodies may consist of a predetermined mixture of antibodies against one or more epitopes are generated in a single cell. Oligoclonal antibodies may comprise a plurality of heavy chains capable of pairing with a common light chain to generate antibodies with multiple specificities (e.g., PCT publication WO 04/009618 which is incorporated by reference herein). Oligoclonal antibodies are particularly useful when it is desired to target multiple epitopes on a single target molecule. Those skilled in the art will know or can determine what type of antibody or mixture of antibodies is applicable for an intended purpose and desired need.

In one embodiment, the antibodies of the invention may be chemically modified (e.g., one or more chemical moieties can be attached to the antibody) or be modified to alter its glycosylation, again to alter one or more functional properties of the antibody. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, each of which is incorporated herein by reference in its entirety.

Additionally or alternatively, an antibody can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342, and U.S. Pat. Nos. 6,946,292 and 7,214,775, each of which is incorporated herein by reference in its entirety.

The present invention also encompasses antibodies that are Fc variants with enhanced antibody dependent cell-mediated cytotoxicity activity. Nonlimiting examples of such Fc variant antibodies are disclosed in U.S. patent application Ser. Nos. 11/203,253 (filed Aug. 15, 2005 and published as U.S. Patent Application Publication No. US 2006/0039904 A1) and 11/203,251 (filed Aug. 15, 2005), and U.S. Provisional Patent Applications 60/674,674 (filed Apr. 26, 2005) and 60/713,711 (filed Sep. 6, 2005), each of which is incorporated by reference herein in its entirety.

The present invention encompasses the use of antibodies or fragments thereof recombinantly fused or chemically conjugated (including both covalent and non-covalent conjugations) to a heterologous agent to generate a fusion protein as both targeting moieties and anti-EphA2 or anti-ErbB2 agents. The heterologous agent may be a polypeptide (or portion thereof, for example, a polypeptide of at least 10, at least 20, at least 30, at least 40, at least 50, at least 60, at least 70, at least 80, at least 90 or at least 100 amino acids), nucleic acid, small molecule (less than 1000 daltons), or inorganic or organic compound. The fusion does not necessarily need to be direct, but may occur through linker sequences. Antibodies fused or conjugated to heterologous agents may be used in vivo to detect, treat, manage, or monitor the progression of a disorder using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties. In some embodiments, the disorder to be detected, treated, managed, or monitored is malignant cancer that overexpresses EphA2, or expresses or overexpresses ErbB2. In other embodiments, the disorder to be detected, treated, managed, or monitored is a pre-cancerous condition associated with cells that overexpress EphA2, or express or overexpress ErbB2. In a specific embodiment, the pre-cancerous condition is high-grade prostatic intraepithelial neoplasia (PIN), fibroadenoma of the breast, fibrocystic disease, or compound nevi.

In one embodiment, enhancing the anti-tumor-potency of antibodies involves linking cytotoxic drugs or toxins to mAbs that are capable of being internalized by a target cell. These agents are termed antibody-drug conjugates (ADCs) and immunotoxins, respectively. Upon administration to a patient, ADCs and immunotoxins bind to target cells via their antibody portions and become internalized, allowing the drugs or toxins to exert their effect. See, for example, WO2007/030642A2, U.S. Patent Appl. Publ. Nos. US2005/0180972 A1, US2005/0123536 A1. See also, for example, Hamblett et al., Clin Canc Res, 10:7063-7070, Oct. 15, 1999, Law et al., Clin Cane Res, 10:7842-7851, Dec. 1, 2004, Francisco et al., Neoplasia, 102(4):1458-1465, Aug. 15, 2003, Russell et al., Clin Cane Res, 11:843-852, Jan. 15, 2005, Doronina et al., Nat Biotech, 21(7):778-784, July 2003, all of which are hereby incorporated by reference herein in their entirety.

In another embodiment, the antibodies of the invention comprise antibody drug conjugates (ADCs) that specifically bind EphA2, such as, but not limited to those disclosed in PCT Publication No. WO 2007/030642, U.S. Provisional Patent Applications 60/714,362 filed on Sep. 7, 2005 and 60/735,966, filed on Nov. 14, 2005, and U.S. patent application Ser. No. 12/065,832, filed Mar. 5, 2008, each of which is incorporated herein by reference in its entirety.

The present invention further includes compositions comprising heterologous agents fused or conjugated to antibody fragments. For example, the heterologous polypeptides may be fused or conjugated to a Fab fragment, Fd fragment, Fv fragment, F(ab)2 fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341 (said references incorporated by reference in their entireties).

Additional fusion proteins, e.g., of any of the antibodies listed in herein, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to EphA2 may be recombined with one or more components, motifs, sections, parts, domains, fragments, etc. of one or more heterologous agents.

In one embodiment, antibodies of the present invention or fragments or variants thereof are conjugated to a marker sequence, such as a peptide, to facilitate purification. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof are conjugated to a diagnostic or detectable agent. Such antibodies can be useful for monitoring or prognosing the development or progression of a cancer as part of a clinical testing procedure, such as determining the efficacy of a particular therapy or as part of a pre-screening procedure. Additionally, such antibodies can be useful for monitoring or prognosing the development or progression of a pre-cancerous condition associated with cells that express or overexpress EphA2 and ErbB2.

Diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luorou; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In other embodiments, antibodies of the present invention or fragments or variants thereof are conjugated to a therapeutic agent such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In one embodiment, the cytotoxic agent is selected from the group consisting of an enediyne, a lexitropsin, a duocarmycin, a taxane, a puromycin, a dolastatin, a maytansinoid, and a vinca alkaloid. In other embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, an auristatin or other dolastatin derivatives, such as auristatin E or auristatin F, AEB, AEVB, AEFP, MMAE (monomethylauristatin E), MMAF (monomethylauristatin F), eleutherobin or netropsin.

In yet other embodiments, the cytotoxic agent of an antibody of the invention is an anti-tubulin agent. In more specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In more specific embodiments, the cytotoxic agent is vincristine, vinblastine, vindesine, vinorelbine, VP-16, camptothecin, paclitaxel, docetaxel, epithilone A, epithilone B, nocodazole, coichicine, colcimid, estramustine, cemadotin, discodermolide, maytansine, DM-1, an auristatin or other dolastatin derivatives, such as auristatin E or auristatin F, AEB, AEVB, AEFP, MMAE (monomethylauristatin E), MMAF (monomethylauristatin F), eleutherobin or netropsin.

In a specific embodiment, the cytotoxic agent of an antibody of the invention is MMAE. In another specific embodiment, the cytotoxic agent of an antibody of the invention is AEFP. In a further specific embodiment, the cytoxic agent of an antibody of the invention is MMAF. Further examples of toxins, spacers, linkers, stretchers and the like, and their structures can be found in U.S. Patent Application Publication Nos. 2006/0074008 A1, 2005/0238649 A1, 2005/0123536 A1, 2005/0180972 A1, 2005/0113308 A1, 2004/0157782 A1, U.S. Pat. No. 6,884,869 B2, U.S. Pat. No. 5,635,483, all of which are hereby incorporated herein in their entirety.

As discussed herein, the drugs used for conjugation to the antibodies of the present invention can include conventional chemotherapeutics, such as doxorubicin, paclitaxel, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the antibodies using the conditionally stable linkers to form potent immunoconjugates.

In certain embodiments, the antibodies of the invention comprise drugs that are at least 40-fold more potent than doxorubicin on EphA2-expressing cells or on ErbB2 expressing cells. Such drugs include, but are not limited to: DNA minor groove binders, including enediynes and lexitropsins, duocarmycins, taxanes (including paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epithilone A and B, estramustine, cryptophysins, cemadotin, maytansinoids, dolastatins, e.g., auristatin E, dolastatin 10, MMAE, MMAF, discodermolide, eleutherobin, and mitoxantrone.

In certain specific embodiments, an antibody of the invention comprises an enediyne moiety. In a specific embodiment, the enediyne moiety is calicheamicin. Enediyne compounds cleave double stranded DNA by generating a diradical via Bergman cyclization.

In other specific embodiments, the cytotoxic or cytostatic agent is auristatin E or an auristatin F, or a derivative thereof. In a further embodiment, the auristatin E derivative is an ester formed between auristatin E and a keto acid. For example, auristatin E can be reacted with paraacetyl benzoic acid or benzoylvaleric acid to produce AEB and AEVB, respectively. Other auristatin derivatives include MMAE, MMAF, and AEFP.

The synthesis and structure of auristatin E, also known in the art as dolastatin-10, and its derivatives are described in U.S. Patent Application Publ. Nos. 2003/0083263 A1 and 2005/0009751 A1; in the International Patent Application No.: PCT/US02/13435, in U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, all of which are incorporated by reference in their entireties herein.

In specific embodiments, the drug is a DNA minor groove binding agent. Examples of such compounds and their syntheses are disclosed in U.S. Pat. No. 6,130,237, which is incorporated by reference in its entirety herein. In certain embodiments, the drug is a CBI compound.

In certain embodiments of the invention, an antibody of the invention comprises an anti-tubulin agent. Anti-tubulin agents are a well established class of cancer therapy compounds. Examples of anti-tubulin agents include, but are not limited to, taxanes (e.g., Taxol® (paclitaxel), docetaxel), T67 (Tularik), vincas, and auristatins (e.g., auristatin E, AEB, AEVB, MMAE, MMAF, AEFP). Anti-tubulin agents included in this class are also: vinca alkaloids, including vincristine and vinblastine, vindesine and vinorelbine; taxanes such as paclitaxel and docetaxel and baccatin derivatives, epithilone A and B, nocodazole, fluorouracil and colcimid, estramustine, cryptophysins, cemadotin, maytansinoids, combretastatins, dolastatins, discodermolide and eleutherobin.

In a specific embodiment, the drug is a maytansinoid, a group of anti-tubulin agents. In a more specific embodiment, the drug is maytansine. Further, in a specific embodiment, the cytotoxic or cytostatic agent is DM-1 (ImmunoGen, Inc.; see also Chari et al. 1992, Cancer Res 52:127-131). Maytansine, a natural product, inhibits tubulin polymerization resulting in a mitotic block and cell death. Thus, the mechanism of action of maytansine appears to be similar to that of vincristine and vinblastine. Maytansine, however, is about 200 to 1,000-fold more cytotoxic in vitro than these vinca alkaloids. In another specific embodiment, the drug is an AEFP.

In certain specific embodiments of the invention, the drug is not a polypeptide of greater than 50, 100 or 200 amino acids, for example a toxin. In a specific embodiment of the invention, the drug is ricin.

In other specific embodiments of the invention, an antibody of the invention does not comprise one or more of the cytotoxic or cytostatic agents the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, vinca alkaloids, purine antagonists, and dihydrofolate reductase inhibitors. In more specific embodiments, the high potency drug is not one or more of an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, fluorouracil, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine. VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, and trifluridine.

In certain embodiments, the cytotoxic or cytostatic agent is a dolastatin. In more specific embodiments, the dolastatin is of the auristatin class. In a specific embodiment of the invention, the cytotoxic or cytostatic agent is MMAE. In another specific embodiment of the invention, the cytotoxic or cytostatic agent is AEFP. In another specific embodiment of the invention, the cytotoxic or cytostatic agent is MMAF.

In other embodiments, antibodies of the present invention or fragments or variants thereof are conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGF (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-4 (“IL-4”), interleukin-6 (“IL-6”), interleukin-7 (“IL-7”), interleukin-9 (“IL-9”), interleukin-15 (“IL-15”), interleukin-12 (“IL-12”), granulocyte macrophage colony stimulating factor (“GM-CSF”), and granulocyte colony stimulating factor (“G-CSF”)), or a growth factor (e.g., growth hormone (“GH”)).

In other embodiments, antibodies of the present invention or fragments or variants thereof are conjugated to a therapeutic agent such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules, further discussed herein below, are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to antibodies are well known, see, e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to a moiety does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216, each of which is incorporated herein by reference in its entirety.

Two approaches may be taken to minimize drug activity outside the cells that are targeted by the antibodies of the invention: first, an antibody that binds to cell membrane, but not soluble, EphA2 or ErbB2 may be used, so that the drug, including drug produced by the actions of the prodrug converting enzyme, is concentrated at the cell surface of the activated lymphocyte. Another approach for minimizing the activity of drugs bound to the antibodies of the invention is to conjugate the drugs in a manner that would reduce their activity unless they are hydrolyzed or cleaved off the antibody. Such methods would employ attaching the drug to the antibodies with linkers that are sensitive to the environment at the cell surface of the activated lymphocyte (e.g., the activity of a protease that is present at the cell surface of the activated lymphocyte) or to the environment inside the activated lymphocyte the conjugate encounters when it is taken up by the activated lymphocyte (e.g., in the endosomal or, for example by virtue of pH sensitivity or protease sensitivity, in the lysosomal environment). Examples of linkers that can be used in the present invention are disclosed in U.S. Patent Application Publication Nos. 2005/0123536 A1, 2005/0180972 A1, 2005/0113308 A1, 2004/0157782 A1, and U.S. Pat. No. 6,884,869 B2, all of which are hereby incorporated by reference herein in their entirety. Alternatively, an antibody can be conjugated to a second antibody to form an antibody heteroconjugate as described by Segal in U.S. Pat. No. 4,676,980, which is incorporated herein by reference in its entirety.

In one embodiment, a recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence may be based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane.

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95:9184-9). In this embodiment, the polypeptides can be linked by a peptide bond or a non peptide bond (e.g. with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington's Pharmaceutical Sciences, 18th Ed., E. W. Martin (ed.), Mack Publishing Co., Easton, Pa. (1990)). If a particular cell type in vivo is to be targeted, for example, by regional perfusion of an organ or section of artery/blood vessel, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

### 7.2.2 Nucleic Acid Molecules

Nucleic acid molecules specific for EphA2 or ErbB2, particularly those that inhibit or encode one or more moieties that inhibit EphA2 or ErbB2 expression, can also be used in methods of the invention. The present invention encompasses antisense nucleic acid molecules, i.e., molecules which are complementary to all or part of a sense nucleic acid encoding EphA2 or ErbB2, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids. Antisense nucleic acid molecules may be determined by any method known in the art, using the nucleotide sequences in publicly available databases such as GenBank. For example, using the nucleotide sequence of human EphA2 (fore example, GenBank accession no. NM_{—}004431.2) or the nucleotide sequence of human ErbB2 (for example, GenBank accession no. M95667.1).

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, e.g., EphA2 or ErbB2).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or peptides which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter may be used.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

### 7.2.3 Ribozymes

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes; described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding EphA2 or ErbB2 can be designed based upon the nucleotide sequence of EphA2 or ErbB2. For example, a derivative of a Tetrahymena L-19 IVS RNA can be constructed in which the nucleotide sequence of the active site is complementary to the nucleotide sequence to be cleaved in U.S. Pat. Nos. 4,987,071 and 5,116,742. Alternatively, an mRNA encoding a polypeptide of the invention can be used to select a catalytic RNA having a specific ribonuclease activity from a pool of RNA molecules. See, e.g., Bartel and Szostak, 1993, Science 261:1411.

### 7.2.4 RNA Interference

In certain embodiments, an RNA interference (RNAi) molecule is used to inhibit EphA2 or ErbB2 expression or activity. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of EphA2 to produce a phenotype that is the same as that of a null mutant of EphA2 (Wianny & Zernicka-Goetz, 2000, Nature Cell Biology 2: 70-75). Nonlimiting examples of siRNA in the context of receptor tyrosine kinases can be found in U.S. Patent Application Publication No. 2005/0246794.

### 7.2.5 MicroRNA

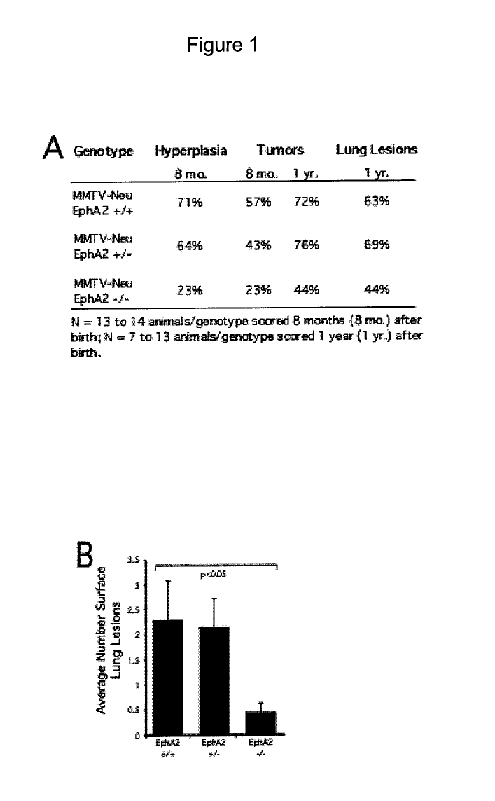

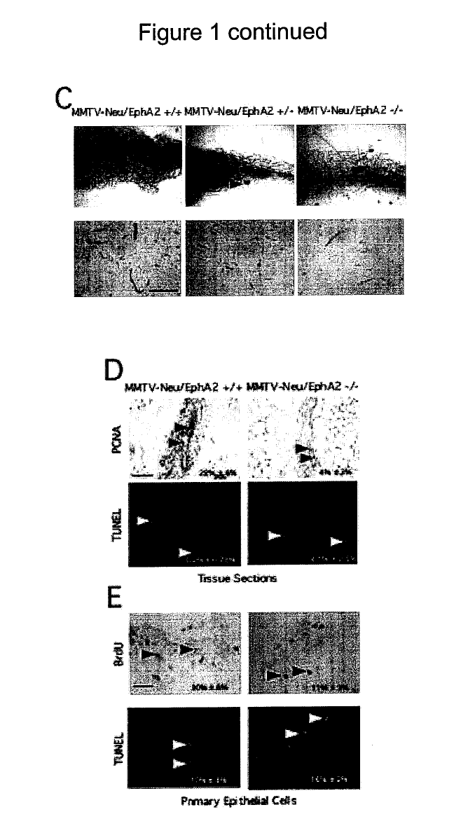

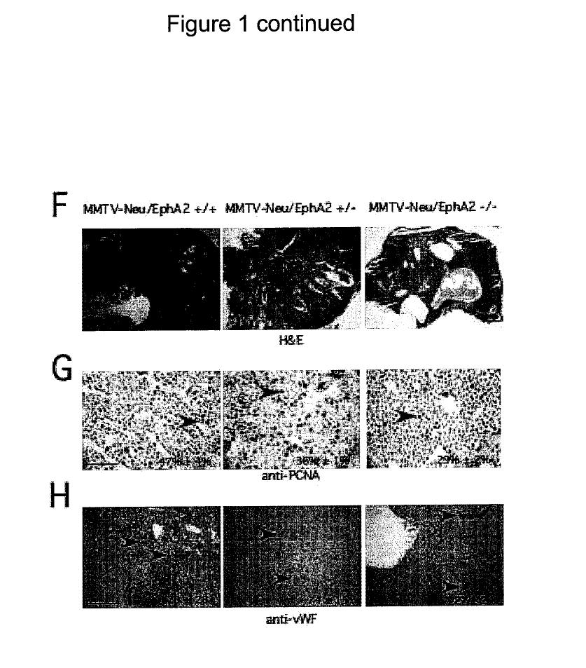

Micro RNAs (referred to as “miRNAs”) are small non-coding RNAs, belonging to a class of regulatory molecules found in plants and animals that control gene expression by binding to complementary sites on target messenger RNA (mRNA) transcripts (FIG. 1) miRNAs are generated from large RNA precursors (termed pri-miRNAs) that are processed in the nucleus into approximately 70 nucleotide pre-miRNAs, which fold into imperfect stem-loop structures (Lee, Y., et al., Nature (2003) 425(6956):415-9) (FIG. 1). The pre-miRNAs undergo an additional processing step within the cytoplasm where mature miRNAs of 18-25 nucleotides in length are excised from one side of the pre-miRNA hairpin by an RNase III enzyme, Dicer (Hutvagner, G., et al., Science (2001) 12:12 and Grishok, A., et al., Cell (2001) 106(1):23-34) miRNAs have been shown to regulate gene expression in two ways. First, miRNAs that bind to protein-coding mRNA sequences that are exactly complementary to the miRNA induce the RNA-mediated interference (RNAi) pathway. Messenger RNA targets are cleaved by ribonucleases in the RISC complex. This mechanism of miRNA-mediated gene silencing has been observed mainly in plants (Hamilton, A. J. and D. C. Baulcombe, Science (1999) 286(5441):950-2 and Reinhart, B. J., et al., MicroRNAs in plants. Genes and Dev. (2002) 16:1616-1626), but an example is known from animals (Yekta, S., I. H. Shih, and D. P. Bartel, Science (2004) 304(5670):594-6). In the second mechanism, miRNAs that bind to imperfect complementary sites on messenger RNA transcripts direct gene regulation at the posttranscriptional level but do not cleave their mRNA targets. mRNAs identified in both plants and animals use this mechanism to exert translational control of their gene targets (Bartel, D. P., Cell (2004) 116(2):281-97). In certain embodiments, the EphA2 and/or ErbB2 targeting agents are miRNA's. Nonlimiting examples of miRNA's can be found in U.S. Patent Application Publication No. 2006/0189557.

### 7.2.6 Aptamers

In specific embodiments, the invention provides aptamers of EphA2 or ErbB2.

As is known in the art, aptamers are macromolecules composed of nucleic acid (e.g., RNA, DNA) that bind tightly to a specific molecular target (e.g., EphA2 or ErbB2 proteins, EphA2 or ErbB2 polypeptides and/or EphA2 or ErbB2 epitopes as described herein). A particular aptamer may be described by a linear nucleotide sequence and is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind to EphA2 or ErbB2 or a fragment thereof can be achieved through methods known in the art. For example, aptamers can be selected using the SELEX (Systematic Evolution of Ligands by Exponential Enrichment) method (Tuerk and Gold, 1990, Science 249:505-510, which is incorporated by reference herein in its entirety). In the SELEX method, a large library of nucleic acid molecules (e.g., 1015 different molecules) is produced and/or screened with the target molecule (e.g., EphA2 or ErbB2 proteins, EphA2 or ErbB2 polypeptides and/or EphA2 or ErbB2 epitopes or fragments thereof as described herein). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology, the entire teachings of which are incorporated herein by reference).

In particular embodiments, the aptamers of the invention have the binding specificity and/or functional activity described herein for the antibodies of the invention. Thus, for example, in certain embodiments, the present invention is drawn to aptamers that have the same or similar binding specificity as described herein for the antibodies of the invention (e.g., binding specificity for EphA2 or ErbB2 polypeptide, fragments of vertebrate EphA2 or ErbB2 polypeptides, epitopic regions of vertebrate EphA2 or ErbB2 polypeptides (e.g., epitopic regions of EphA2 or ErbB2 that are bound by the antibodies of the invention). In particular embodiments, the aptamers of the invention can bind to an EphA2 or ErbB2 polypeptide and inhibit one or more activities of the EphA2 or ErbB2 polypeptide.

### 7.2.7 Avimers

In one embodiment, the invention provides an anti-EphA2 or anti-ErbB2 avimer (Avidia, Inc.). Avimers are a new class of therapeutic proteins that are from human origin, are unrelated to antibodies and antibody fragments, are composed of several modular and reusable binding domains, have individual binding domains with a molecular weight of 4.5 kD each, have a molecular weight that typically ranges between 9 kD and 18 kD, can be designed to exert either antagonistic or agonistic activity, can bind with high affinity to multiple epitopes simultaneously, have sub-nM binding affinity (Kd) and blocking function (IC50), are non-glycosylated and can be produced by microbial expression, exhibit class behavior during production, can be produced at high yields, can be delivered by subcutaneous injection, have excellent tissue penetration, have a customized pharmacokinetic profile, and are non-immunogenic in animals (see, for example, U.S. Patent Application Publ. Nos. 2005/0221384, 2005/0164301, 2005/0053973 and 2005/0089932, 2005/0048512, and 2004/0175756, each of which is hereby incorporated by reference herein in its entirety).

### 7.2.8 Gene Therapy

In a specific embodiment, nucleic acids that alter EphA2 or ErbB2 expression (e.g., EphA2 or ErbB2 antisense nucleic acids or EphA2 or ErbB2 dsRNA) are administered to treat, prevent or manage a hyperproliferative disease, particular cancer, by way of gene therapy. Gene therapy refers to therapy performed by the administration to a subject of an expressed or expressible nucleic acid. In this embodiment of the invention, the antisense nucleic acids are produce and mediate a prophylactic or therapeutic effect.

Any of the methods for gene therapy available in the art can be used according to the present invention. Exemplary methods are described below. For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In one embodiment, a composition of the invention comprises EphA2 or ErbB2 nucleic acids that reduce EphA2 or ErbB2 expression, said nucleic acids being part of an expression vector that expresses the nucleic acid in a suitable host. In particular, such nucleic acids have promoters, for example, heterologous promoters, said promoter being inducible or constitutive, and, optionally, tissue-specific. In another particular embodiment, nucleic acid molecules are used in which the nucleic acid that reduces EphA2 or ErbB2 expression and any other desired sequences are flanked by regions that promote homologous recombination at a desired site in the genome, thus providing for intrachromosomal expression of the nucleic acids that reduce EphA2 or ErbB2 expression (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

### 7.2.9 Other Kinase Inhibitors

In one embodiment, other kinase inhibitors that are capable of inhibiting or reducing the expression of EphA2 or ErbB2 can be used in methods of the invention. Such kinase inhibitors include, but are not limited to, inhibitors of Ras, and inhibitors of certain other oncogenic receptor tyrosine kinases such as EGFR and ErbB2. Non-limiting examples of such inhibitors are disclosed in U.S. Pat. Nos. 6,462,086; 6,130,229; 6,638,543; 6,562,319; 6,355,678; 6,656,940; 6,653,308; 6,642,232, and 6,635,640, and U.S. Patent Application No. 2006/0252056, each of which is incorporated herein by reference in its entirety. In a particular embodiment, the kinase inhibitors inhibit or reduce EphA2 or ErbB2 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., RT-PCR, a Northern blot or an immunoassay such as an ELISA, Western blot).

In a specific embodiment, the methods of the invention encompass administration of a composition of the invention in combination with the administration of one or more prophylactic/therapeutic agents that are inhibitors of kinases such as, but not limited to, ABL, ACK, AFK, AKT (e.g., AKT-1, AKT-2, and AKT-3), ALK, AMP-PK, ATM, Aurora1, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, MK, PDGF receptor alpha, PDGF receptor beta, PHK, PI-3 kinase, PICA, PKB, PKC, PKG, PRK1, PYK2, p38 kinases, p135tyk2, p34cdc2, p42cdc2, p42mapk, p44mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). In certain embodiments, an antibody of the invention is administered in combination with the administration of one or more prophylactic/therapeutic agents that are inhibitors of RTK's (e.g., EphA2 or ErbB2). In one embodiment, an antibody of the invention is administered in combination with the administration of one or more prophylactic/therapeutic agents that are inhibitors of EphA2 and/or ErbB2.

In another specific embodiment, the methods of the invention encompass administration of a composition of the invention in combination with the administration of one or more prophylactic/therapeutic agents that are angiogenesis inhibitors such as, but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CM; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-β); Vasculostatin; Vasostatin (calreticulin fragment); ZD6126; ZD6474; farnesyl transferase inhibitors (FTI); and bisphosphonates.

In another specific embodiment, the methods of the invention encompass administration of a composition of the invention in combination with the administration of one or more prophylactic/therapeutic agents that are anti-cancer agents such as, but not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti cancer drugs include, but are not limited to: 20 epi 1,25 dihydroxyvitamin D3, 5 ethynyluracil, abiraterone, aclarubicin, acylfulvene, adecypenol, adozelesin, aldesleukin, ALL TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didenmin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofuran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. Additional anti-cancer drugs are 5-fluorouracil and leucovorin.

The invention also encompasses administration of the compositions of the invention in combination with radiation therapy comprising the use of x-rays, gamma rays and other sources of radiation to destroy the cancer cells. In certain embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (61st ed., 2007).

### 7.3 Delivery Methods and Vehicles

The present invention provides methods and compositions designed for treatment, management, or prevention of a hyperproliferative cell disease, particularly cancer. To enhance the therapeutic or prophylactic effects of anti-EphA2 or ErbB2 agents or other anti-cancer agents, and/or to decrease the unwanted side effects of such agents, the methods and compositions of the invention target certain types of cells or specific tissues, particularly cells expressing or overexpressing EphA2 and ErbB2.

Any delivery vehicle known in the art can be used in accordance with the present invention. Various delivery systems are known and can be used to administer one or more compositions of the invention, e.g., encapsulation in liposomes, microparticles, microcapsules, recombinant cells capable of expressing the antibody or antibody fragment, receptor-mediated endocytosis (see, e.g., Wu and Wu, 1987, J. Biol. Chem. 262:4429-4432), construction of a nucleic acid as part of a retroviral or other vector, etc. For example, nucleic acid molecules can be delivered by use of microparticle bombardment (e.g., a gene gun; Biolistic, Dupont), or coating with lipids or transfecting agents that are conjugated to an EphA2 or ErbB2 targeting moiety, encapsulation in liposomes, microparticles, or microcapsules, or by administering them in linkage to a peptide which is known to enter the nucleus, or by administering it in linkage to a ligand subject to receptor-mediated endocytosis (see, e.g., Wu and Wu, 1987, J. Biol. Chem. 262:4429) (which can be used to target cell types specifically expressing the receptors), etc.

In a specific embodiment, the nucleic acid sequences are directly administered in vivo. This can be accomplished by any of numerous methods known in the art, e.g., by constructing them as part of an appropriate nucleic acid expression vector (e.g., vectors as described above and target to EphA2 or ErbB2) and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or using any delivery vehicles known in the art and targeting EphA2 or ErbB2 by conjugated to an appropriated targeting moiety or by administering them in linkage to a peptide which is known to enter the nucleus, by administering it in linkage to a ligand subject to receptor-mediated endocytosis (see, e.g., Wu and Wu, 1987, J. Biol. Chem. 262:4429) (which can be used to target cell types specifically expressing the receptors, e.g., EphA2 or ErbB2), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor, for example, EphA2 or ErbB2. Alternatively, the nucleic acid can be introduced intracellularly and incorporated within host cell DNA for expression, by homologous recombination (Koller and Smithies, 1989, PNAS USA 86:8932; and Zijlstra et al., 1989, Nature 342:435).

In one embodiment, the nucleic acid is introduced into a cell prior to administration in vivo of the resulting recombinant cell. Such introduction can be carried out by any method known in the art, including but not limited to, transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and heritable and expressible by its cell progeny.

The resulting recombinant cells can be delivered to a subject by various methods known in the art. The amount of cells envisioned for use depends on the desired effect, patient state, etc., and can be determined by one skilled in the art.

A delivery vehicle may target certain type of cells, e.g., by virtue of an innate feature of the vehicle, or by a moiety conjugated to the vehicle, which moiety specifically binds a particular subset of cells, e.g., by binding to a cell surface molecule characteristic of the subset of cells to be targeted. In one embodiment, a delivery vehicle of the invention targets cells expressing EphA2 and/or ErbB2, and may target cells expressing EphA2 and/or ErbB2 not bound to a ligand over EphA2 and/or ErbB2 bound to a ligand. In a specific embodiment, an EphA2 or ErbB2 targeting moiety is attached to a delivery vehicle of the invention.

The delivery vehicle can be, for example, a peptide vector, a peptide-DNA aggregate, a liposome, a gas-filled microsome, an encapsulated macromolecule, a nanosuspension, and the like (see e.g., Torchilin, Drug Targeting. Eur. J. Phamaceutical Sciences: v. 11, pp. S81-S91 (2000); Gerasimov, Boomer, Qualls, Thompson, Cytosolic drug delivery using pH- and light-sensitive liposomes, Adv. Drug Deliv. Reviews: v. 38, pp. 317-338 (1999); Hafez, Cullis, Roles of lipid polymorphism in intracellular delivery, Adv. Drug Deliv. Reviews: v. 47, pp. 139-148 (2001); Hashida, Akamatsu, Nishikawa, Fumiyoshi, Takakura, Design of polymeric prodrugs of prostaglandin E1 having galactose residue for hepatocyte targeting, J. Controlled Release: v. 62, pp. 253-262 (1999); Shah, Sadhale, Chilukuri, Cubic phase gels as drug delivery systems, Adv. Drug Deliv. Reviews: v. 47, pp. 229-250 (2001); Muller, Jacobs, Kayser, Nanosuspensions as particulate drug formulations in therapy: Rationale for development and what we can expect for the future, Adv. Drug Delivery Reviews: v. 47, pp. 3-19 (2001)). In some embodiments, the delivery vehicle is a viral vector. In a specific embodiment, a delivery vehicle can be, for example, an HVJ (Sendai virus)-liposome gene delivery system (see e.g., Kaneda et al., Ann. N.Y. Acad. Sci. 811:299-308 (1997)); a “peptide vector” (see e.g., Vidal et al., CR Acad. Sci III 32:279-287 (1997)); a peptide-DNA aggregate (see e.g., Niidome et al., J. Biol. Chem. 272:15307-15312 (1997)); lipidic vector systems (see e.g., Lee et al., Crit Rev Ther Drug Carrier Syst. 14:173-206 (1997)); polymer coated liposomes (Marin et al., U.S. Pat. No. 5,213,804; Woodle et al., U.S. Pat. No. 5,013,556); cationic liposomes (Epand et al., U.S. Pat. No. 5,283,185; Jessee, J. A., U.S. Pat. No. 5,578,475; Rose et al, U.S. Pat. No. 5,279,833; Gebeyehu et al., U.S. Pat. No. 5,334,761); gas filled microspheres (Unger et al., U.S. Pat. No. 5,542,935), or encapsulated macromolecules (Low et al., U.S. Pat. No. 5,108,921; Curiel et al., U.S. Pat. No. 5,521,291; Groman et al., U.S. Pat. No. 5,554,386; Wu et al., U.S. Pat. No. 5,166,320) (all references are incorporated herein by reference in their entireties).

Methods of packaging the therapeutic or prophylactic agent(s) into a delivery vehicle depend on various factors, such as the type of the delivery vehicle being used, or the hydrophobic or hydrophilic nature of the agent(s). Any packaging method known in the art can be used in the present invention.

### 7.3.1 Viruses

Viruses are attractive delivery vehicles for their natural ability to infect host cells and introduce foreign nucleic acids. Viral vector systems useful in the practice of the instant invention include, for example, naturally occurring or recombinant viral vector systems. For example, viral vectors can be derived from the genome of human or bovine adenoviruses, vaccinia virus, herpes virus, adeno-associated virus (see e.g., Xiao et al., Brain Res. 756:76-83 (1997), minute virus of mice (MVM), HIV, HPV and HPV-like particles, sindbis virus, and retroviruses (including but not limited to Rous sarcoma virus), and MoMLV, hepatitis B virus (see e.g., Ji et al., J. Viral Hepat. 4:167-173 (1997)). Typically, genes of interest are inserted into such vectors to allow packaging of the gene construct, typically with accompanying viral DNA, followed by infection of a sensitive host cell and expression of the gene of interest. One example of a recombinant viral vector is the adenoviral vector delivery system which has a deletion of the protein IX gene (see, International Patent Application WO 95/11984, which is herein incorporated by reference in its entirety). Another example of a recombinant viral vector is the recombinant parainfluenza virus vector (recombinant PIV vectors, disclosed in e.g., International Patent Application Publication No. WO 03/072720, MedImmune Vaccines, Inc., incorporated herein by reference in its entirety) or a recombinant metapneumovirus vector (recombinant MPV vectors, disclosed in e.g., International Patent Application Publication No. WO 03/072719, MedImmune Vaccines, Inc., incorporated herein by reference in its entirety).

In some instances it may be advantageous to use vectors derived from a different species from that which is to be treated in order to avoid the preexisting immune response. For example, equine herpes virus vectors for human gene therapy are described in WO 98/27216 published Aug. 5, 1998. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in WO 97/06826 published Apr. 10, 1997, which is incorporated herein by reference.

The virus can be replication competent (e.g., completely wild-type or essentially wild-type such as Ad d1309 or Ad d1520), conditionally replicating (designed to replicate under certain conditions) or replication deficient (substantially incapable of replication in the absence of a cell line capable of complementing the deleted functions). Alternatively, the viral genome can possess certain modifications to the viral genome to enhance certain desirable properties such as tissue selectivity. For example, deletions in the E1a region of adenovirus result in preferential replication and improved replication in tumor cells. The viral genome can also be modified to include therapeutic transgenes. The virus can possess certain modifications to make it “selectively replicating,” i.e. that it replicates preferentially in certain cell types or phenotypic cell states, e.g., cancerous. For example, a tumor or tissue specific promoter element can be used to drive expression of early viral genes resulting in a virus which preferentially replicates only in certain cell types. Alternatively, one can employ a pathway-selective promoter active in a normal cell to drive expression of a repressor of viral replication. Selectively replicating adenoviral vectors that replicate preferentially in rapidly dividing cells are described in International Patent Application Nos. WO 990021451 and WO 990021452, each of which is incorporated herein by reference.

In a specific embodiment, viral vectors that contain nucleic acid sequences that reduce EphA2 or ErbB2 expression and/or function are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in accordance with the present invention are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in delivering nucleic acid molecules of the invention. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses as a delivery vehicle can be found in Rosenfeld et al., 1991, Science 252:431; Rosenfeld et al., 1992, Cell 68:143; Mastrangeli et al., 1993, J. Clin. Invest. 91:225; International Publication No. WO94/12649; and Wang et al., 1995, Gene Therapy 2:775. In one embodiment, adenovirus vectors are used.

Adeno-associated virus (AAV) has also been proposed for use as a delivery vehicle (Walsh et al., 1993, Proc. Soc. Exp. Biol. Med. 204:289-300; and U.S. Pat. No. 5,436,146).

A variety of approaches to create targeted viruses have been described in the literature. For example, cell targeting has been achieved with adenovirus vectors by selective modification of the viral genome knob and fiber coding sequences to achieve expression of modified knob and fiber domains having specific interaction with unique cell surface receptors, e.g., engineered to contain an EphA2 or ErbB2 targeting moiety. Examples of such modifications are described in Wickham et al. (1997) J. Virol. 71(11):8221-8229 (incorporation of RGD peptides into adenoviral fiber proteins); Arnberg et al. (1997) Virology 227:239-244 (modification of adenoviral fiber genes to achieve tropism to the eye and genital tract); Harris and Lemoine (1996) TIG 12(10):400-405; Stevenson et al. (1997) J. Virol. 71(6):4782-4790; Michael et al. (1995) Gene Therapy 2:660-668 (incorporation of gastrin releasing peptide fragment into adenovirus fiber protein); and Ohno et al. (1997) Nature Biotechnology 15:763-767 (incorporation of Protein A-IgG binding domain into Sindbis virus).

Other methods of cell specific targeting rely on the conjugation of antibodies or antibody fragments to the envelope proteins (see e.g. Michael et al. (1993) J. Biol. Chem. 268:6866-6869, Watkins et al. (1997) Gene Therapy 4:1004-1012; Douglas et al. (1996) Nature Biotechnology 14: 1574-1578). For example, an antibody or an antibody fragment that binds EphA2 or ErbB2 can be chemically conjugated to the surface of the virion by modification of amino acyl side chains in the antibody (particularly through lysine residues). Another non-limiting example of decorating the surface of a virus for targeting purpose is demonstrated in the U.S. Pat. No. 6,635,476, which is incorporated herein by reference. Alternative to the use of antibodies, others have complexed targeting proteins to the surface of the virion. See, e.g. Nilson et al. (1996) Gene Therapy 3:280-286 (conjugation of EGF to retroviral proteins).

In some embodiments, an EphA2 or ErbB2 targeting moiety, e.g., an anti-EphA2 or ErbB2 antibody, an EphA2 or ErbB2 ligand, a peptide or other targeting moieties known in the art, is attached to the surface of the virus, and thus direct the virus to the cells that are expressing or overexpressing EphA2 and/or ErbB2.

### 7.3.2 Synthetic Vectors

Non-viral synthetic vectors can also be used as a delivery vehicle in accordance with the present invention. For examples, a targeting moiety can be attached to a polycation (e.g., lipid or polymer) backbone. The polycation backbone also forms a complex with the therapeutic or prophylactic agent (e.g., a nucleic acid molecule) to be delivered. A non-limiting example of such delivery vehicle is polylysine, which has been conjugated to a diverse set of ligands that selectively target particular receptors on certain cell types. See e.g., Cotton et al., Proc. Natl. Acad. Sci. 87:4033-4037 (1990); Fur et al., Receptor-mediated targeted gene delivery using asialoglycoprotein-polylysine conjugates, in Gene Therapeutics: Methods and Applications of Direct Gene Transfer, Wolff JA Ed, Birkhauser: Boston, pp 382-390 (1994); McGraw et al., Internalization and sorting of macromolecules: Endocytosis, in Targeted Drug Delivery, Juliano R L ed., Springer: New York, pp 11-41 (1991); and Uike et al., Biosci Biotechnol. Biochem. 62:1247-1248 (1998). In some embodiments, an EphA2 or ErbB2 targeting moiety, e.g., an anti-EphA2 or ErbB2 antibody, an EphA2 or ErbB2 ligand, a peptide or other targeting moieties known in the art, is attached to the polycation backbone (e.g., polylysine), and thereby directs the therapeutic agent(s) to the cells that express or overexpress EphA2 and/or ErbB2.

Chimeric multi-domain peptides can also be used as delivery vehicles in accordance with the present invention. See e.g., Fominaya et al., J. Biol. Chem. 271:10560-10568 (1996); and Uherek et al., J. Biol. Chem. 273:8835-8841 (1998). Such carrier incorporates targeting (i.e., EphA2 or ErbB2), endosomal escape, and DNA binding motifs into a single synthetic peptide molecule.

In accordance with the present invention, liposomes can be used as a delivery vehicle. Liposomes are closed lipid vesicles used for a variety of therapeutic purposes, and in particular, for carrying therapeutic or prophylactic agents to a target region or cell by systemic administration of liposomes. Liposomes are usually classified as small unilamellar vesicles (SUV), large unilamellar vesicles (LUV), or multi-lamellar vesicles (MLV). SUVs and LUVs, by definition, have only one bilayer, whereas MLVs contain many concentric bilayers. Liposomes may be used to encapsulate various materials, by trapping hydrophilic molecules in the aqueous interior or between bilayers, or by trapping hydrophobic molecules within the bilayer. Gangliosides are believed to inhibit nonspecific adsorption of serum proteins to liposomes, thereby prevent nonspecific recognition of liposomes by macrophages.

In particular, liposomes having a surface grafted with chains of water-soluble, biocompatible polymer, such as polyethylene glycol, have become important drug carries. These liposomes offer an extended blood circulation lifetime over liposomes lacking the polymer coating. The grafted polymer chains shield or mask the liposome, thus minimizing nonspecific interaction by plasma proteins. This in turn slows the rate at which the liposomes are cleared or eliminated in vivo since the liposome circulate unrecognized by macrophages and other cells of the reticuloendothelial system. Furthermore, due to the so-called enhanced permeability and retention effect, the liposomes tend to accumulate in sites of damaged or expanded vasculature, e.g., tumors, and sites of inflammation.

It would be desirable to formulate a liposome composition having a long blood circulation lifetime and capable of retaining an entrapped drug for a desired time, yet able to release the drug on demand. One approach described in the art for achieving these features has been to formulate a liposome from a non-vesicle-forming lipid, such as dioleoylphosphatidylethanolamine (DOPE), and a lipid bilayer stabilizing lipid, such as methoxy-polyethylene glycol-distearoyl phosphatidylethanolamine (mPEG-DSPE) (Kirpotin et al., FEBS Lett. 388:115-118 (1996)). In this approach, the mPEG is attached to the DSPE via a cleavable linkage. Cleavage of the linkage destabilizes the liposome for a quick release of the liposome contents.

Labile bonds for linking PEG polymer chains to liposomes have been described (U.S. Pat. Nos. 5,013,556, 5,891,468; WO 98/16201). The labile bond in these liposome compositions releases the PEG polymer chains from the liposomes, for example, to expose a surface attached targeting ligand or to trigger fusion of the liposome with a target cell.

In a liposomal drug delivery system, an anti-EphA2 or ErbB2 is entrapped during liposome formation and then administered to the patient to be treated. See e.g., U.S. Pat. Nos. 3,993,754, 4,145,410, 4,224,179, 4,356,167, and 4,377,567. In the present invention, a liposome is modified to have one or more EphA2 or ErbB2 targeting moieties on its surface.

### 7.3.3 Hybrid Vectors

Hybrid vectors exploit endosomal escape capabilities of viruses in combination with the flexibility of non-viral vectors. Hybrid vectors can be divided into two subclasses: (1) membrane disrupting particles, either virus particles or other fusogenic peptides, added as separate entities in conjunction with non-viral vectors; and (2) such particles combined into a single complex with a traditional non-viral vector.

For example, a hybrid vector may use adenovirus in trans with a targeted non-viral vector, for example, adenovirus together with complexes of transferrin/polylysine, antibody/polylysine, or asialoglycoprotein/polylysine. See e.g., Cotton et al., Proc. Natl. Acad. Sci. 89:6094-6098 (1992); Curiel et al., Receptor-mediated gene delivery employing adenovirus-polylysine-DNA complexes, in Gene Therapeutics: Methods and Applications of Direct Gene Transfer, Wolff J A ed., Birkhauser: Boston, pp 99-116 (1994); Wagner et al., Proc. Natl. Acad. Sci. 89:6099-6103 (1992); Christiano et al., Proc. Natl. Acad. Sci. 90:2122-2126 (1993); each of which is incorporated herein by reference in its entirety. The mechanism of action of such hybrid vectors begins with the specific binding of both targeted complex and virus particle to their respective receptors. Upon binding, targeted complex and virus particle can either be internalized in the same vesicle or into separate endosomes. In a specific embodiment, a viral particle is directly conjugated to a targeted vector. Incorporation of viral particles into targeted complexes can be done, e.g., through streptavidin/biotinylation of adenovirus and polylysine, through antibodies pre-coupled to polylysine, or through direct chemical conjugation. See e.g., Verga et al., Biotechnology and Bioengineering 70(6): 593-605 (2000). In certain embodiments, the present invention provides hybrid vectors comprising one or more EphA2 or ErbB2 targeting moieties.

### 7.4 Prophylactic/Therapeutic Methods

The present invention encompasses methods for treating, preventing, or managing a disease or disorder associated with expression or overexpression of EphA2 and ErbB2 and/or a cell hyperproliferative disorder, particularly cancer, in a subject comprising administering an effective amount of a composition that can target cells expressing EphA2 and ErbB2, and altering the EphA2 and/or ErbB2 expression or function, and/or having therapeutic or prophylactic effects on the hyperproliferative cell disease. In one embodiment, the method of the invention comprises administering to a subject a composition comprising an EphA2 or ErbB2 targeting moiety attached to a therapeutic or prophylactic agent against the hyperproliferative cell disease. In another embodiment, the method of the invention comprises administering to a subject a composition comprising a nucleic acid comprising a nucleotide sequence encoding an EphA2 or ErbB2 targeting moiety and a nucleotide sequence encoding a therapeutic or prophylactic agent against the hyperproliferative disease. In another embodiment, the method of the invention comprises administering to a subject a composition comprising an EphA2 or ErbB2 targeting moiety and a nucleic acid comprising a nucleotide sequence encoding a therapeutic or prophylactic agent against the hyperproliferative disease, wherein the targeting moiety is associated with the nucleic acid either directly or through a delivery vector for delivery to cells expressing or overexpressing EphA2 and/or ErbB2. In specific embodiments, an EphA2 or ErbB2 targeting moiety also inhibits EphA2 or ErbB2 expression or activity.

The present invention encompasses methods for treating, preventing, or managing a disease or disorder associated with expression or overexpression of EphA2 and/or ErbB2 and/or a cell hyperproliferative disorder, for example, cancer, in a subject comprising administering one or more antibodies that target EphA2 or ErbB2 and/or alter EphA2 or ErbB2 expression or activity, wherein said antibodies comprise EphA2 or ErbB2 agonistic or antagonistic antibodies, EphA2 or ErbB2 intrabodies, or EphA2 or ErbB2 cancer cell phenotype inhibiting antibodies or exposed EphA2 or ErbB2 epitope antibodies or EphA2 or ErbB2 antibodies that bind EphA2 with a K_{off} less than 3×10⁻¹s⁻¹, or EphA2 or ErbB2 avimers. In a specific embodiment, the disorder to be treated, prevented, or managed is malignant cancer. In another specific embodiment, the disorder to be treated, prevented, or managed is a pre-cancerous condition associated with cells that express or overexpress EphA2 or ErbB2. In more specific embodiments, the pre-cancerous condition is high-grade prostatic intraepithelial neoplasia (PIN), fibroadenoma of the breast, fibrocystic disease, or compound nevi.

In one embodiment, the compositions of the invention can be administered in combination with one or more other therapeutic agents useful in the treatment, prevention or management of diseases or disorders associated with EphA2 or ErbB2 expression or overexpression, hyperproliferative disorders, and/or cancer. In certain embodiments, one or more compositions of the invention are administered to a mammal, preferably a human, concurrently with one or more other therapeutic agents useful for the treatment of cancer. The term “concurrently” is not limited to the administration of prophylactic or therapeutic agents at exactly the same time, but rather it is meant that the compositions of the invention and the other agent are administered to a subject in a sequence and within a time interval such that the compositions of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In other embodiments, the compositions of the invention are administered before, concurrently to, or after surgery. Preferably the surgery completely removes localized tumors or reduces the size of large tumors. Surgery can also be done as a preventive measure or to relieve pain.

The invention provides methods for treating, preventing, and managing a disease or disorder associated with EphA2 or ErbB2 expression or overexpression and/or hyperproliferative cell disease, particularly cancer, by administrating to a subject in need thereof a therapeutically or prophylactically effective amount of one or more compositions of the invention. In another embodiment, the compositions of the invention can be administered in combination with one or more other therapeutic agents. The subject is preferably a mammal such as non-primate (e.g., cows, pigs, horses, cats, dogs, rats, etc.) and a primate (e.g., monkey, such as a cynomolgous monkey and a human). In a specific embodiment, the subject is a human.

Specific examples of cancers that can be treated by the methods encompassed by the invention include, but are not limited to, cancers that express or overexpress EphA2 and/or ErbB2. In a further embodiment, the cancer is of an epithelial origin. Examples of such cancers are cancer of the lung, colon, prostate, breast, and skin. Other cancers include cancer of the bladder and pancreas and renal cell carcinoma and melanoma. Additional cancers are listed by example and not by limitation herein below. In particular embodiments, methods of the invention can be used to treat and/or prevent metastasis from primary tumors.

The methods and compositions of the invention comprise the administration of one or more compositions of the invention to subjects/patients suffering from or expected to suffer from cancer, e.g., have a genetic predisposition for a particular type of cancer, have been exposed to a carcinogen, or are in remission from a particular cancer. As used herein, “cancer” refers to primary or metastatic cancers. Such patients may or may not have been previously treated for cancer. The methods and compositions of the invention may be used as a first line or second line cancer treatment. Included in the invention is also the treatment of patients undergoing other cancer therapies and the methods and compositions of the invention can be used before any adverse effects or intolerance of these other cancer therapies occurs. The invention also encompasses methods for administering one or more compositions of the invention to treat or ameliorate symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased.

In particular embodiments, the compositions of the invention are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis. In a specific embodiment, compositions of the invention are administered to patients with increased levels of the cytokine IL-6, which has been associated with the development of cancer cell resistance to different treatment regimens, such as chemotherapy and hormonal therapy. In another specific embodiment, compositions of the invention are administered to patients suffering from breast cancer that have a decreased responsiveness or are refractory to tamoxifen treatment. In another specific embodiment, compositions of the invention are administered to patients with increased levels of the cytokine IL-6, which has been associated with the development of cancer cell resistance to different treatment regimens, such as chemotherapy and hormonal therapy.

In alternate embodiments, the invention provides methods for treating patients' cancer by administering one or more compositions of the invention in combination with any other treatment or to patients who have proven refractory to other treatments but are no longer on these treatments. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more compositions of the invention are administered to prevent the recurrence of cancer.

In certain embodiments, the existing treatment is chemotherapy. In particular embodiments, the existing treatment includes administration of chemotherapies including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc. Among these patients are patients treated with radiation therapy, hormonal therapy and/or biological therapy/immunotherapy. Also among these patients are those who have undergone surgery for the treatment of cancer.

Alternatively, the invention also encompasses methods for treating patients undergoing or having undergone radiation therapy. Among these are patients being treated or previously treated with chemotherapy, hormonal therapy and/or biological therapy/immunotherapy. Also among these patients are those who have undergone surgery for the treatment of cancer.

In other embodiments, the invention encompasses methods for treating patients undergoing or having undergone hormonal therapy and/or biological therapy/immunotherapy. Among these are patients being treated or having been treated with chemotherapy and/or radiation therapy. Also among these patients are those who have undergone surgery for the treatment of cancer.

Additionally, the invention also provides methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

In other embodiments, the invention provides administration of one or more compositions of the invention without any other cancer therapies for the treatment of cancer, but who have proved refractory to such treatments. In specific embodiments, patients refractory to other cancer therapies are administered one or more compositions of the invention in the absence of cancer therapies.

In other embodiments, patients with a pre-cancerous condition associated with cells that express or overexpress EphA2 and ErbB2 can be administered compositions of the invention to treat the disorder and decrease the likelihood that it will progress to malignant cancer. In a specific embodiment, the pre-cancerous condition is high-grade prostatic intraepithelial neoplasia (PIN), fibroadenoma of the breast, fibrocystic disease, or compound nevi.

In yet other embodiments, the invention provides methods of treating, preventing and managing non-cancer hyperproliferative cell disorders, particularly those associated with expression or overexpression of EphA2 and ErbB2, including but not limited to, asthma, chromic obstructive pulmonary disorder (COPD), restenosis (smooth muscle and/or endothelial), psoriasis, etc. These methods include methods analogous to those described above for treating, preventing and managing cancer, for example, by administering the compositions of the invention, as well as combination therapy, administration to patients refractory to particular treatments, etc.

### 7.5 Cancers

Cancers and related disorders that can be treated, prevented, or managed by methods and compositions of the present invention include but are not limited to cancers of an epithelial cell origin. Examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to luoroura, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

In some embodiments, the cancer is malignant and expresses or overexpresses EphA2 and ErbB2. In other embodiments, the disorder to be treated is a pre-cancerous condition associated with cells that overexpress EphA2 and ErbB2.

In other embodiments, the methods and compositions of the invention are used for the treatment and/or prevention of breast, colon, ovarian, lung, and prostate cancers and melanoma and are provided below by example rather than by limitation.

### 7.6 Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. In one embodiment, the composition of the invention further comprises an additional therapeutic, e.g., anti-cancer, agent.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), or MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is an example of a carrier that can be used when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Methods of administering a prophylactic or therapeutic agent of the invention include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, prophylactic or therapeutic agents of the invention are administered intramuscularly, intravenously, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic agent can be delivered in a controlled release or sustained release system. In one embodiment, a pump may be used to achieve controlled or sustained release (see Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179 189; Song et al., 1995, PDA Journal of Pharmaceutical Science & Technology 50:372 397; Cleek et al., 1997, Pro. Int'l. Symp. Control. Rel. Bioact. Mater. 24:853 854; and Lam et al., 1997, Proc. Int'l. Symp. Control Rel. Bioact. Mater. 24:759 760, each of which is incorporated herein by reference in its entirety.

### 7.6.1 Formulations

Pharmaceutical compositions for use in accordance with the present invention may be formulated in conventional manner using one or more physiologically acceptable carriers or excipients. Thus, the compositions of the invention may be formulated for administration by inhalation or insufflation (either through the mouth or the nose) or oral, parenteral or mucosal (such as buccal, vaginal, rectal, sublingual) administration. In one embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachet indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In one embodiment of the invention, the formulation and administration of various chemotherapeutic, biological/immunotherapeutic and hormonal therapeutic agents are known in the art and often described in the Physician's Desk Reference, 56th ed. (2002). For instance, in certain specific embodiments of the invention, the therapeutic agents of the invention can be formulated and supplied as provided herein.

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the compositions of the invention, are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 7.6.2 Dosages and Frequency of Administration

The amount of a therapy (e.g., prophylactic or therapeutic agent) or a composition of the invention which will be effective in the prevention, treatment, management, and/or amelioration of a hyperproliferative disease or one or more symptoms thereof can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, management, and/or amelioration of an hyperproliferative disease or one or more symptoms thereof can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physician's Desk Reference (61st ed., 2007).

In various embodiments, the therapies (e.g., prophylactic or therapeutic agents) are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In certain embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (58th ed., 2004).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient is typically 0.0001 mg/kg to 100 mg/kg of the patient's body weight. The dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In a specific embodiment, the dosage of antibodies administered to prevent, treat, manage, and/or ameliorate a hyperproliferative disease or one or more symptoms thereof in a patient is 150 mg/kg or less, 125 μg/kg or less, 100 mg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the antibodies of the invention administered to prevent, treat, manage, and/or ameliorate a hyperproliferative disease, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of an effective amount of one or therapies (e.g., therapeutic or prophylactic agents) of the invention, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the therapies (e.g., therapeutic or prophylactic agents) of the invention. In yet other embodiments, a subject is administered a dose of an effective amount of one or antibodies of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies and a subsequent dose of an effective amount of one or more antibodies of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of preventing, treating, managing, or ameliorating a hyperproliferative disease or one or more symptoms thereof, said method comprising administering to a subject in need thereof a dose of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more therapies (e.g., therapeutic or prophylactic agents), combination therapies, or compositions of the invention. In another embodiment, the invention provides a method of preventing, treating, managing, and/or ameliorating a hyperproliferative disease or one or more symptoms thereof, said methods comprising administering to a subject in need thereof a dose of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more antibodies, combination therapies, or compositions of the invention once every 3 days, once every 4 days, once every 5 days, once every 6 days, once every 7 days, once every 8 days, once every 10 days, once every two weeks, once every three weeks, or once a month.

The present invention provides methods of preventing, treating, managing, or preventing cancer or a hyperproliferative disease or one or more symptoms thereof, said method comprising: (a) administering to a subject in need thereof one or more doses of a prophylactically or therapeutically effective amount of one or more antibodies, combination therapies, or compositions of the invention; and (b) monitoring the plasma level/concentration of the said administered antibodies in said subject after administration of a certain number of doses of the said therapies (e.g., therapeutic or prophylactic agents). Moreover, said certain number of doses is 1, 2, 3, 4, 5, 6, 7, 8, 9, 10, 11, or 12 doses of a prophylactically or therapeutically effective amount one or more antibodies, compositions, or combination therapies of the invention.

In a specific embodiment, the invention provides a method of preventing, treating, managing, and/or ameliorating cancer or a hyperproliferative disease or one or more symptoms thereof, said method comprising: (a) administering to a subject in need thereof a dose of at least 10 μg (for example, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more therapies (e.g., therapeutic or prophylactic agents) of the invention; and (b) administering one or more subsequent doses to said subject when the plasma level of the antibody administered in said subject is less than 0.1 μg/ml, less than 0.25 μg/ml, less than 0.5 μg/ml, less than 0.75 μg/ml, or less than 1 μg/ml. In another embodiment, the invention provides a method of preventing, treating, managing, and/or ameliorating cancer or a hyperproliferative disease or one or more symptoms thereof, said method comprising: (a) administering to a subject in need thereof one or more doses of at least 10 μg (at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more antibodies of the invention; (b) monitoring the plasma level of the administered antibodies in said subject after the administration of a certain number of doses; and (c) administering a subsequent dose of antibodies of the invention when the plasma level of the administered antibody in said subject is less than 0.1 μg/ml, less than 0.25 μg/ml, less than 0.5 μg/ml, less than 0.75 μg/ml, or less than 1 μg/ml. Said certain number of doses is 1, 2, 3, 4, 5, 6, 7, 8, 9, 10, 11, or 12 doses of an effective amount of one or more antibodies of the invention.

Therapies (e.g., prophylactic or therapeutic agents), other than the antibodies of the invention, which have been or are currently being used to prevent, treat, manage, and/or ameliorate a hyperproliferative disease or one or more symptoms thereof can be administered in combination with one or more antibodies according to the methods of the invention to treat, manage, prevent, and/or ameliorate a hyperproliferative disease or one or more symptoms thereof. The dosages of prophylactic or therapeutic agents used in combination therapies of the invention are lower than those which have been or are currently being used to prevent, treat, manage, and/or ameliorate a hyperproliferative disease or one or more symptoms thereof. The recommended dosages of agents currently used for the prevention, treatment, management, or amelioration of a hyperproliferative disease or one or more symptoms thereof can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physician's Desk Reference (PDR) 58th ed., 2004, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same antibody of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an antibody of the invention may be repeated and the administration may be separated by at least at least 1 day, 2 days, 3 days, 5 days, 10 days, 15 days, 30 days, 45 days, 2 months, 75 days, 3 months, or at least 6 months.

### 7.7 Selecting Patient Populations

EphA2 or ErbB2 can also serve as markers for cancer or precancerous conditions. In the present invention, Applicants have found that EphA2 and ErbB2 interact with each other in the cancerous condition. Accordingly, in one embodiment, the invention provides a method for screening or selecting patient populations, for diagnosing a cancerous or precancerous condition, or staging a cancer, by detecting and, optionally, quantifying the presence, amount or activity of EphA2 and ErbB2 in a biological sample. The diagnostic method of the invention can be used to obtain or confirm an initial diagnosis of cancer, or to provide information on cancer localization, cancer metastasis, or cancer prognosis. In a specific embodiment, provided is a diagnostic kit that is optimized to screen and detect both EphA2 and ErbB2.

In one embodiment of the diagnostic method, a biological sample such as a tissue, organ or fluid is removed from the mammal, cells are lysed, and the lysate is contacted with a polyclonal or monoclonal EphA2 and/or ErbB2 antibody. The resulting antibody complex is either itself detectable or capable of associating with another compound to form a detectable complex. Bound antibody can be detected directly in an ELISA or similar assay; alternatively, the diagnostic agent can comprise a detectable label, and the detectable label can be detected using methods known in the art.

In embodiments of the method wherein EphA2 and ErbB2 are detected via the binding of a detectably labeled diagnostic agent such as an antibody, labels include chromogenic dyes, fluorescent labels and radioactive labels. Among the most commonly used chromagens are 3-amino-9-ethylcarbazole (AEC) and 3,3′-diaminobenzidine tetrahydrocholoride (DAB). These can be detected using light microscopy.

The most commonly used fluorescent labeling compounds are fluorescein isothiocyanate, rhodamine, phycoerythrin, phycocyanin, allophycocyanin, o phthaldehyde and fluorescamine. Chemiluminescent and bioluminescent compounds such as luminol, isoluminol, theromatic acridinium ester, imidazole, acridinium salt, oxalate ester, luciferin, luciferase, and aequorin also may be used. When the fluorescent labeled antibody is exposed to light of the proper wavelength, its presence can be detected due to its fluorescence.

Radioactive isotopes which are particularly useful for labeling the antibodies of the present invention include ³H, ¹²⁵I, ¹³¹I, ³⁵S, ³²P, and ¹⁴C. The radioactive isotope can be detected by such means as the use of a gamma counter, a scintillation counter, or by autoradiography.

Antibody-antigen complexes can be detected using western blotting, dot blotting, precipitation, agglutination, enzyme immunoassay (ETA) or enzyme linked immunosorbent assay (ELISA), immunohistochemistry, in situ hybridization, flow cytometry on a variety of tissues or bodily fluids, and a variety of sandwich assays. These techniques are well known in the art. See, for example, U.S. Pat. No. 5,876,949, hereby incorporated by reference. In an enzyme immunoassay (EIA) or enzyme linked immunosorbent assay (ELISA), the enzyme, when subsequently exposed to its substrate, reacts with the substrate and generates a chemical moiety which can be detected, for example, by spectrophotometric, fluorometric, or visual means. Enzymes which can be used to detectably label antibodies include, but are not limited to malate dehydrogenase, staphylococcal nuclease, delta 5 steroid isomerase, yeast alcohol dehydrogenase, alpha glycerophosphate dehydrogenase, triose phosphate isomerase, horseradish peroxidase, alkaline phosphatase, asparaginase, glucose oxidase, beta galactosidase, ribonuclease, urease, catalase, glucose 6 phosphate dehydrogenase, glucoamylase, and acetylcholinesterase. Other methods of labeling and detecting antibodies are known in the art and are within the scope of this invention.

In another embodiment of the diagnostic method, a biological sample is subjected to a biochemical assay for EphA2 and ErbB2 kinase activity. Detection can also be accomplished by employing a detectable reagent that binds to DNA or RNA coding for the EphA2 and ErbB2 proteins.

EphA2 and ErbB2 can be used as a marker for cancer, precancerous or metastatic disease in a wide variety of tissue samples, including biopsied tumor tissue and a variety of body fluid samples, such as blood, plasma, spinal fluid, saliva, and urine.

Other antibodies may be used in combination with antibodies that bind to EphA2 and ErbB2 to provide further information concerning the presence or absence of cancer and the state of the disease. For example, the use of phosphotyrosine-specific antibodies provides additional data for determining detecting or evaluating malignancies.

### 7.8 Characterization and Demonstration of Therapeutic Utility

Toxicity and efficacy of the prophylactic and/or therapeutic protocols of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the SCID mouse model or transgenic mice where a mouse EphA2 and/or ErbB2 is replaced with the human EphA2 and/or ErbB2, nude mice with human xenografts or any animal model (including hamsters, rabbits, etc.) known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

The protocols and compositions of the invention can be tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed, e.g., altered phosphorylation/degradation of EphA2, inhibition of or decrease in growth and/or colony formation in soft agar or tubular network formation in three-dimensional basement membrane or extracellular matrix preparations. A lower level of proliferation or survival of the contacted cells indicates that the therapeutic agent is effective to treat the condition in the patient. Alternatively, instead of culturing cells from a patient, therapeutic agents and methods may be screened using cells of a tumor or malignant cell line. Many assays standard in the art can be used to assess such survival and/or growth; for example, cell proliferation can be assayed by measuring 3H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, altered phosphorylation/degradation of EphA2, decreased growth and/or colony formation in soft agar or tubular network formation in three-dimensional basement membrane or extracellular matrix preparation, etc.

Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of cancer.

### 7.9 Kits

The invention provides a pharmaceutical pack or kit comprising one or more containers filled with a composition of the invention. Additionally, one or more other prophylactic or therapeutic agents useful for the treatment of a cancer can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The invention further provides screening and/or diagnostic kits related to detecting EphA2 and/or ErbB2. In a specific embodiment, the invention provides a kit that is optimized to screen and detect both EphA2 and ErbB2, sequentially or simultaneously.

Whereas, particular embodiments of the invention have been described above for purposes of description, it will be appreciated by those skilled in the art that numerous variations of the details may be made without departing from the invention as described in the appended claims.

All publications, patents and patent applications mentioned in this specification are herein incorporated by reference into the specification to the same extent as if each individual publication, patent or patent application was specifically and individually indicated to be incorporated herein by reference.

### 8. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 8.1 Example 1

Reagents: Antibodies against the following proteins were used: EphA2 (Zymed Laboratories, Burlingame, Calif.; Santa Cruz Biotechnology; Santa Cruz, Calif.; Upstate Biotechnology, Lake Placid, N.Y.), EphA4 (Upstate Biotechnology), proliferating cell nuclear antigen (PCNA; BD Biosciences, Franklin Lakes, N.J.), anti-Erk, anti-phosphothreonine-202/tyrosine-204 Erk, Akt, phosphoserine-473 Akt (Cell Signaling Technology, Boston, Mass.), anti-tubulin (Sigma-Aldrich, St. Louis, Mo.), ErbB2 (Neomarkers/Lab Vision Corporation, Fremont, Calif.), anti-b-actin (Santa Cruz Biotechnology), Ras (BD Biosciences), RhoA (Santa Cruz Biotechnology; BD Biosciences), von Willebrand factor (vWF; Zymed Laboratories, South San Francisco, Calif.), E-cadherin (BD Biosciences), Ki67 (Vision Biosystems Inc., Norwell, Mass.), and normal rabbit IgG (Santa Cruz Biotechnology). Therapeutic anti-EphA2 (1C1) and control (R347) antibodies were provided by MedImmune, Inc. (Gaithersburg, Md.). Raf-1 RBD agarose Ras assay reagent was purchased from Upstate Biotechnology. 5-Bromo-2′-deoxyuridine (BrdU) was purchased from Sigma-Aldrich. BrdU detection and ApopTag Red In Situ Apoptosis kits were purchased from Zymed Laboratories and Chemicon/Millipore (Billerica, Mass.), respectively. Avidin peroxidase reagents were from Vector Laboratories (Burlingame, Calif.), and liquid 3,3′-diaminobenzidine tetrahydrochloride (DAB) substrate kit was from Zymed Laboratories. Ephrin-A1-Fc was from R&D Systems (Minneapolis, Minn.). Estrogen, progesterone, insulin, and epidermal growth factor (EGF) were from Sigma-Aldrich. 4′,6-diamidino-2 phenylindole dihydrochloride (DAPI) was purchased from Sigma-Aldrich. TO-PRO-3 iodide nuclear stain, CellTracker orange CMTMR and CellTracker green CMFDA dyes was purchased from Molecular Probes (Invitrogen Corporation, Carlsbad, Calif.). Growth factor-reduced Matrigel was purchased from BD Biosciences. AG825 ErbB2 kinase inhibitor was from Calbiochem (EMD Biosciences, San Diego, Calif.). Recombinant adenoviruses expressing constitutively active RhoA (Q63L) and Erk-1 were purchased from Cell Biolabs (San Diego, Calif.) and Vector Biolabs (Philadelphia, Pa.), respectively. Control adenoviruses expressing b-galactosidase and adenoviruses expressing EphA2 have been previously described (Brantley-Sieders et al., 2004; Cheng and Chen, 2001).

Mice and In Vivo Tumor Studies: All animals were housed under pathogen-free conditions, and experiments were performed in accordance with AAALAC guidelines and with Vanderbilt University Institutional Animal Care and Use Committee approval. EphA2-deficient mice were backcrossed with FVB animals for 5 to 7 generations prior to crossing with MMTV-Neu or MMTV-PyV-mT mice on an inbred FVB background [Jackson Laboratories, Bar Harbor, Me.; (Guy et al., 1992a; Guy et al., 1992b)]. MMTV-Neu or MMTV-PyV-mT positive transgenic animals that were wild-type, heterozygous, or null for ephA2 (Brantley-Sieders et al., 2004b) were identified by polymerase chain reaction (PCR) analysis of genomic DNA from tail biopsy using the following primers: 5′-GGG TGC CAA AGT AGA ACT GCG-3′ (forward) (SEQ ID NO:1), 5′-GAC AGA ATA AAA CGC ACG GGT G-3′ (neo) (SEQ ID NO:2), 5′-TTC AGC CAA GCC TAT GTA GAA AGC-3′ (reverse) (SEQ ID NO:3). The neu and PyV-mT transgenes were detected by PCR using primers and conditions recommended by Jackson Laboratories. Age-matched littermates were monitored for tumor formation by weekly palpation.

Tumors and lungs were collected from two cohorts of MMTV-Neu hemizygous, EphA2 +/+, +/−, and −/− animals at 8 months and 1 year after birth. Tumor and lungs were collected from MMTV-PyV-mT hemizygous, EphA1 +/+, +/−, and −/− animals 100 days after birth. Tumors were enumerated and dimensions measured by caliper. Tumor volume was calculated using the following formula: volume=length×width 2×0.52 (Bergers et al., 2000). Lungs were fixed, dehydrated, and surface metastases enumerated. For transplantation studies, the left inguinal mammary gland fat pad of 3 week old recipient EphA2 +/+ or EphA2 FVB female animals was cleared of endogenous epithelium as described previously (Brantley et al., 2001), and injected with 10⁶ cells tumor cells derived from MMTV-Neu (Muraoka et al., 2003) or MMTV-PyV-mT (Muraoka-Cook et al., 2004) animals. Resulting tumors were harvested 4 to 5 weeks after injection for analysis. Where indicated, beginning at 2 weeks after tumor cell injection, recipient mice received intraperitoneal injections of 1C1 anti-EphA2 antibody or control IgG (10 mg/kg twice weekly for 3 weeks), prior to collection and analysis of primary tumors. At least 10 animals/condition were analyzed in 2-3 independent experiments.

Histologic Analyses: Mammary glands and tumors were harvested at indicated time points and fixed in 10% neutral buffered formalin (Fisher Scientific, Hampton, N.H.) for 24 hours at 4° C. Whole-mount hematoxylin staining of mammary glands and hematoxylin and eosin staining of 7 mm mammary gland tissue sections was performed as described previously (Brantley et al., 2001). For EphA2 immunohistochemistry antigen retrieval was performed by boiling in citrate buffer and sections were probed with 5 mg/ml rabbit anti-EphA2 antibody (Zymed Laboratories) as described previously (Brantley et al., 2002). Immunohistochemical staining for PCNA was performed as described previously (Brantley et al., 2002), and proliferation was quantified by calculating the average percentage of PCNA positive nuclei relative to total nuclei (4 random 20× fields of ³4 independent mammary and tumor samples per genotype). Apoptosis assays were performed using the Apoptag red in situ apoptosis detection kit as per manufacturer's protocol. Apoptosis was calculated as the average percentage TUNEL positive nuclei relative to total nuclei (4 random 20× fields of ³4 independent mammary and tumor samples per genotype). Phospho-Erk was detected in tissue sections using rabbit monoclonal anti-P-Erk antibody clone 20G11 as per Cell Signaling Technology's protocol. Colorimetric immunohistochemical staining for von Willebrand Factor (vWF) was performed by the Vanderbilt University Immunohistochemistry Core Facility, and immunofluorescence staining was performed as described previously (Brantley-Sieders et al., 2005). Microvascular density was determined by counting the number of vWF-positive vessels in 4 random 20× fields/sample of at least 4 tumors/genotype. ErbB2 immunohistochemistry was performed as described above for anti-EphA2 using 5 mg/ml rabbit anti-ErbB2 antibody (Neomarkers/Lab Vision Corporation).

Cell Culture: Primary mammary epithelial cells (PMECs) were isolated from mice as described previously (Brantley et al., 2001; Muraoka et al., 2002) and maintained in PMEC media [DMEM/F12 media (Mediatech, Herndon, Va.) supplemented with 5 ng/ml estrogen, 5 ng/ml progesterone, 5 ng/ml EGF, and 5 mg/ml insulin (all purchased from Sigma-Aldrich)] on growth factor-reduced Matrigel (1/20 dilution) coated tissue culture dishes. Primary tumor cells were derived from wild-type or EphA2-deficient MMTV-Neu animals as previously described (Muraoka et al., 2003). Briefly, tumor tissue was dispersed in PMEC digestion medium (Brantley et al., 2001; Muraoka et al., 2002). The cells were pelleted, washed with DMEM/F12 plus 10% fetal bovine serum (Hyclone, Logan, Utah), washed several times in serum-free media, the plated in PMEC media. The cell suspension was sequentially plated on tissue culture dishes every 24 hours for three days, and tumor cells were collected and expanded from the day three plating. Enrichment of tumor cells in cultures was verified by expression of the neu transgene (Muraoka et al., 2003). The MMTV-Neu tumor-derived cell line (Muraoka et al., 2003) and the MMTV-PyV-mT tumor-derived cell line (Muraoka-Cook et al., 2004) used in transplantation and signaling studies were cultured in PMEC media. For EphA2 degradation studies, tumor cells were cultured in the presence of 1C1 anti-EphA2 antibody or control IgG (MedImmune) at the indicated concentrations for 48 hours prior to harvesting lysates for immunoblot analysis. In vitro proliferation and apoptosis analyses were performed as described previously (Brantley et al., 2002; Muraoka et al., 2002) using BrdU and TUNEL detection kits described above. For rescue experiments, EphA2-deficient Neu primary tumor cells were transduced with 1×108 pfu/ml adenovirus expressing Erk-1 or control b-galactosidase 48 hours prior to BrdU assay. Transwell migration assays were performed as described previously (Brantley-Sieders et al., 2004b). For rescue experiments, EphA2-deficient Neu primary tumor cells were transduced with 1×108 pfu/ml adenovirus expressing constitutively active RhoA (Q63L) or control b-galactosidase 48 hours prior to transwell assay. Tumor-endothelial cell co-culture migration assays were performed as described previously (Brantley-Sieders et al., 2005; Brantley-Sieders et al., 2006).

siRNA sequences for mouse EphA2 or irrelevant control sequences were cloned into pRetroSuper viral vector and used to produce retroviruses for infection of MMTV-Neu tumor cells as previously described (Brantley-Sieders et al., 2006; Brummelkamp et al., 2002). The following sequences were used to target EphA2: siRNA#1 5′-GCCAAAGTAGAACTGCGTT (1140-1158)-3′ (SEQ ID NO:4); siRNA#2 5′-GCGCTAGACAAGTTCCTTA (2211-2229)-3′ (SEQ ID NO:5); control siRNA 5′-GCACCAGTTCAGCAAGACT-3′ (SEQ ID NO:6). Three dimensional spheroid cultures of were established as described previously (Debnath et al., 2003). Cultures were maintained for eight days prior to photodocumentation. Digital images were scored for spheroid culture area in 4 random fields, 3 cultures/field, using Image J software (National Institutes of Health). For confocal imaging, spheroid cultures were fixed in 10% neutral buffered formalin and subjected to immunohistochemistry for E-cadherin followed by nuclear staining with TO-PRO-3 as previously described (Spancake et al., 1999). Tumor cells were transplanted into the cleared fat pads of recipient FVB mice as described above. At least 10 animals/condition were analyzed in 2-3 independent experiments.

Parental MCF10A and MCF10A cells stably overexpressing human ErbB2/Her2 were maintained as described previously (Ueda et al., 2004). Three dimensional spheroid cultures were established as described previously (Debnath et al., 2003). Cells were transduced with 1×108 pfu/ml adenovirus expressing constitutively EphA2 or control b-galactosidase 48 hours prior to analysis. Staining for confocal analysis was performed as described above.

Immunoprecipitation and Immunoblot Analysis: Immunoprecipitation and immunoblot of EphA2 was performed as described previously (Brantley et al., 2002). ErbB2 was immunoprecipitated using 1 mg rabbit anti-ErbB2 plus 1 mg mouse anti-ErbB2 Ab-17 (Neomarkers/Lab Vision Corporation). Where indicated, 250,000 PMECs (for western analyses) or 2.5 million primary tumor cells (for GTP-Ras and -Rho/Rac pull-down assays) were cultured in DMEM:F12 media plus 2% FBS overnight. Cells were treated ±ephrin-A1-Fc, or EphA2-agonist monoclonal antibody 1C1 at the indicated doses and times. Lysates were harvested and used for immunoblot analysis as described previously (Brantley et al., 2002). Densitometry analysis was performed using NIH Image J software.

For Ras and Rho/Rac pulldown assays, tumor tissue was collected, weighed, mechanically homogenized in phosphate buffered saline (PBS) pelleted, and resuspended in manufacturer recommended assay buffer (Upstate Biotechnology). Approximately 500 mg tumor lysate was used per assay. Ras assays were performed using Raf-1 Ras binding domain-GST assay reagent (Upstate Biotechnology) as per manufacturer's protocol. Rho assays were performed using Rhotekin binding domain-GST reagents as previously described (Fang et al., 2005). For some co-immunoprecipitation assays, COST cells were co-transfected with 1 mg each of myc-tagged erbB2 (pcDNA3-erbB2) and ephA2 (pcDNA3-EphA2) using Lipofectamine 2000 (Invitrogen). Cells were lysed in 1% NP-40 Buffer (10 mM Tris-HCl pH 7.5, 150 mM NaCl, 2 mM EDTA, 1% NP-40 plus 50 mM protease inhibitors). Lysates were used for immunoprecipitation with an anti-myc (Sigma-Aldrich) and anti-EphA2 antibodies (Santa Cruz, sc-924) antibodies. Immune complexes were resolved on SDS-PAGE and western blotted using anti-EphA2 and anti-myc antibodies. EphA2 was immunoprecipitated from MMTV-Neu cells, followed by treatment of half the samples with the 1 mM of the cross-linking agent DTSSP. Immunoprecipitates were subjected to western blot analysis using anti-ErbB2 (Neomarkers, 1:2000). EphA2 and ErbB2 were immunoprecipiated from MCF10A and MCF10A.HER2 cells as described above. Where indicated, cells were incubated with 10 mg/ml AG825 ErbB2 kinase inhibitor for 24 or 48 hours prior to immunoprecipitation.

Results

EphA2-Deficiency Suppresses Mammary Epithelial Hyperplasia, Tumorigenesis, Metastasis, and Vascular Recruitment in MMTV-Neu Mice

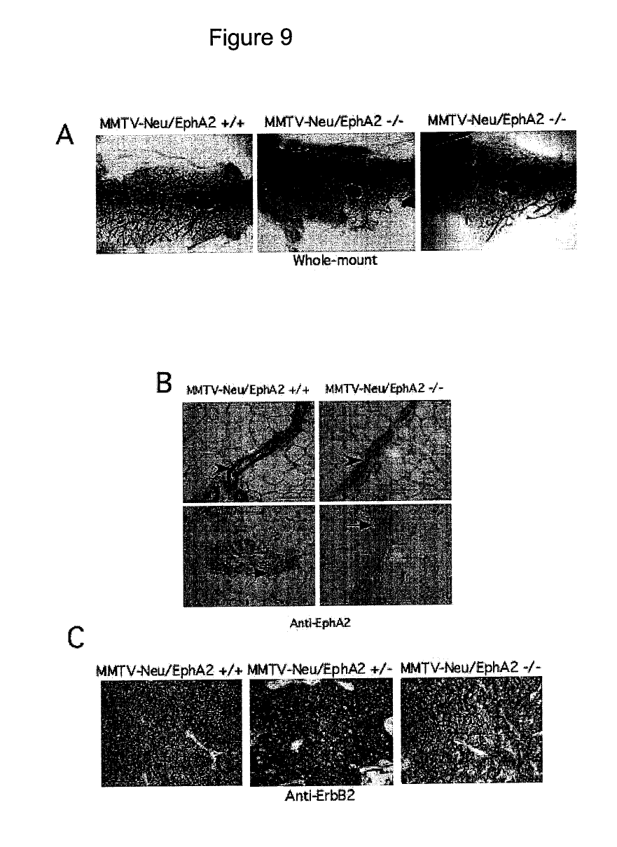

MMTV-Neu positive female mice that were wild-type, heterozygous, or EphA2-deficient were generated and monitored for tumor formation. Mammary gland tissue and/or tumors were collected from two cohorts of animals 8 months and 1 year after birth. Relative to wild-type and heterozygous controls, EphA2-deficient MMTV-Neu females developed epithelial hyperplasias and tumors of the mammary gland with a 2- to 3-fold reduction in frequency (FIG. 1A). Whole-mount and histologic analysis revealed a reduction in mammary epithelial hyperplasia and epithelial cell content for EphA2-deficient MMTV-Neu glands relative to controls (FIG. 1C). No significant change in latency for animals that developed tumors in wild-type versus EphA2-deficient MMTV-Neu animals was observed, though a nearly 3-fold decrease in tumor volume in wild-type versus EphA2-deficient animals was observed. Wild-type and heterozygous controls, however, displayed a higher overall tumor burden compared to EphA2-deficient animals, as they developed two or more tumors 1 year after birth while EphA2-deficient animals developed only single tumors. Moreover, the mammary epithelium failed to penetrate the mammary fat pad in 30% of EphA2-deficient MMTV-Neu mammary glands (FIG. 9A).

To examine pre-malignant changes within the epithelium of EphA2-deficient versus wild-type MMTV-Neu mammary glands, we assessed proliferation and apoptosis in tissue sections by staining for proliferating cell nuclear antigen (PCNA) and by TUNEL assay, respectively. A 5.5-fold reduction in epithelial cell proliferation in the EphA2 −/− versus the EphA2 +/+ MMTV-Neu mammary epithelium was observed, while apoptosis levels were unaffected (FIG. 1D). To determine if proliferation defects were due to EphA2-deficiency in mammary epithelium versus surrounding host tissue, proliferation and apoptosis in purified primary mammary epithelial cells (PMECs) isolated from wild-type or EphA2-deficient animals was analyzed. Proliferation, as measured by incorporation of BrdU, was reduced nearly three-fold in serum-stimulated EphA2-deficient cells relative to wild-type controls (FIG. 1E), suggesting that EphA2-mediated effects on proliferation are, at least in part, intrinsic to the epithelial cell. Interestingly, unlike mammary epithelium in situ, a modest yet significant increase in apoptosis for EphA2-deficient PMEC versus wild-type cells was observed (FIG. 1E). Together, these data indicate that loss of EphA2 inhibits ErbB2-initiated mammary epithelial cell hyperplasia.

Among the EphA2-deficient animals that actually developed tumors, no significant change in latency was observed. However, a nearly 3-fold decrease in tumor volume in EphA2-deficient animals relative to wild-type controls was detected. In addition, wild-type and heterozygous controls displayed a higher overall tumor burden relative to EphA2-deficient mice, as control animals developed two or more tumors 1 year after birth while EphA2-deficient animals developed only single tumors. While EphA2 protein expression was detected in mammary epithelial cells and associated blood vessels in MMTV-Neu wild-type females, EphA2 protein expression was not detected in tissue from EphA2-deficient MMTV-Neu mice (FIG. 9B). Expression levels and localization of ErbB2 in MMTV-Neu tumors were not affected by EphA2-deficiency (FIG. 9C, FIG. 4C). At 1 year of age, lungs harvested from EphA2-deficient MMTV-Neu mice displayed a nearly 5-fold reduction in the number of surface metastases as compared to EphA2 wild-type or heterozygous controls (FIG. 1B). Moreover, the overall frequency of metastasis was decreased in EphA2-deficient animals relative to wild-type and heterozygous controls (FIG. 1A).

Histologic examination of tumors collected from each genotype 8 months after birth disclosed mainly well-circumscribed proliferations of both carcinoma in situ and associated foci of invasive carcinoma with pushing, rather than infiltrating borders. More invasive carcinomas were observed in tumors collected from animals 1 year after birth. Tumors isolated from EphA2-deficient MMTV-Neu mice showed more areas of cystic degeneration and distinct lumen formation, consistent with a more differentiated phenotype relative to the dense, solid sheet-like growth patterns seen in wild-type MMTV-Neu tumors (FIG. 1F). PCNA staining of tumor tissue revealed a nearly 2-fold decrease in proliferation in EphA2-deficient MMTV-Neu tumors relative to wild-type tumors (FIG. 1G). The tumor microvasculature was evaluated by immunohistochemical staining against von Willebrand Factor (vWF), which demonstrated that loss of EphA2 expression is associated with a significant reduction (2.9-fold) in microvascular density (FIG. 1H). Levels of apoptosis were unaltered in EphA2-deficient MMTV-Neu tumors compared to controls.

EphA2 is Required in the Host Microenvironment for Vascular Recruitment in MMTV-Neu Tumors

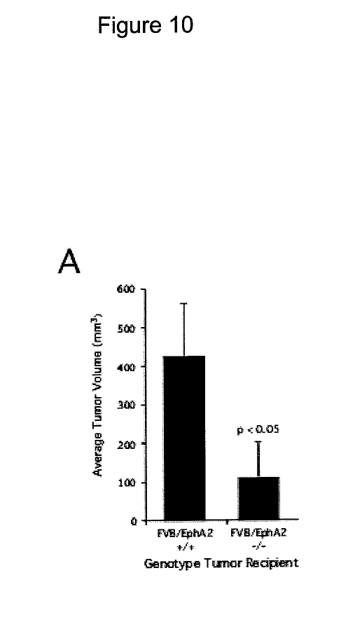

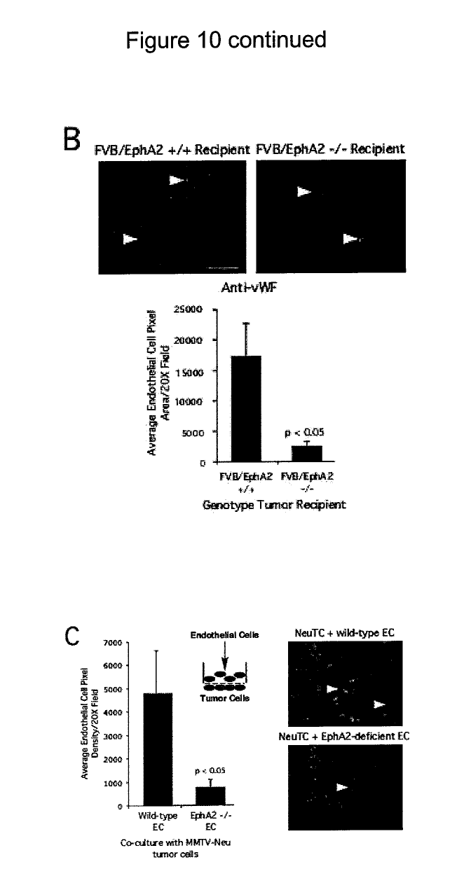

While data presented herein suggests that EphA2-deficiency restrains epithelial proliferation in MMTV-Neu mammary glands, previous data suggests that EphA2 may be required for tumor vascularization [Reviewed in (Brantley-Sieders and Chen, 2004)]. Indeed, decreased tumor vascularization was observed in MMTV-Neu/EphA2-deficient tumors (FIG. 1H). To determine if the defects in tumor microvascular density result from EphA2-deficiency in host tissue versus tumor cells, wild-type MMTV-Neu tumor cells (Muraoka et al., 2003) were orthotopically transplanted into the cleared fat pad of syngeneic wild-type or EphA2-deficient FVB host animals. Wild-type tumor cells transplanted into EphA2-deficient hosts produced significantly smaller tumors than those transplanted into wild-type hosts (FIG. 10A). A 7-fold decrease in microvascular density of tumors isolated from EphA2-deficient recipients versus wild-type control recipients was observed (FIG. 10B). Consistent with these data, microvascular endothelial cells isolated from EphA2-deficient animals displayed a markedly decreased migratory response to MMTV-Neu tumor cells in co-culture assays compared to the robust migratory response exhibited by endothelial cells isolated from wild-type control animals (FIG. 10C). Together, these data suggest that EphA2 signaling promotes tumorigenesis and progression through distinct processes both in the tumor microenvironment, including vascular endothelium, and within the tumor parenchyma.

Loss of EphA2 expression impairs tumor formation and invasiveness in MMTV-Neu tumor cells.

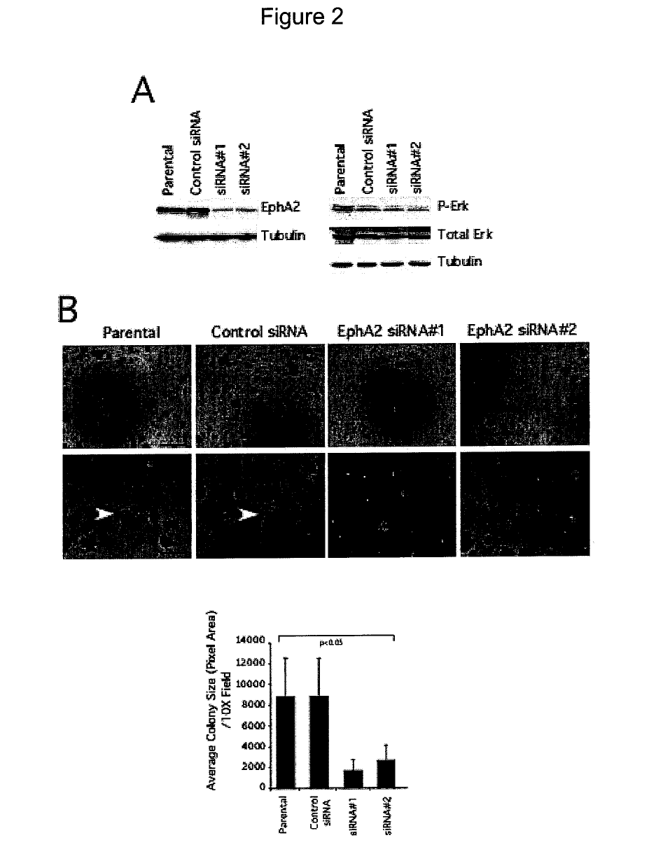

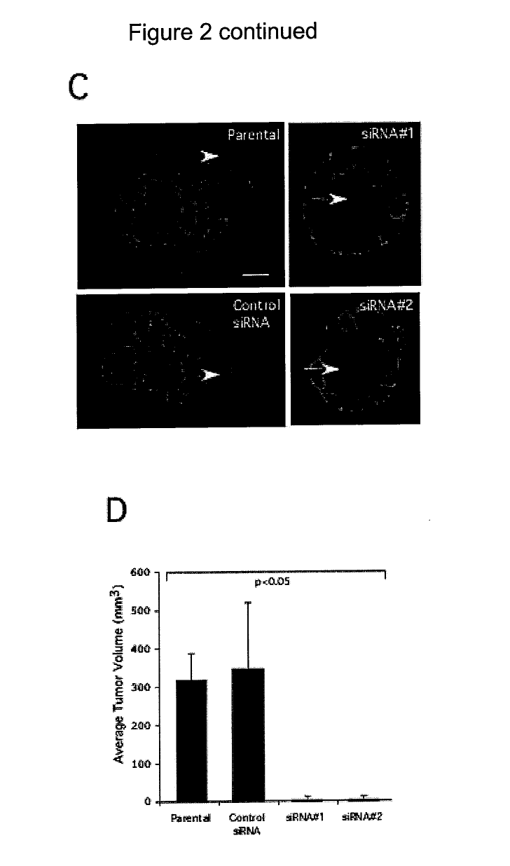

In addition to analysis of EphA2 function in tumorigenesis and progression within endogenous MMTV-Neu tumors in which EphA2-deficiency precedes tumorigenesis, we wished to examine the effects of diminishing EphA2 expression in established tumor cells. An RNAi knockdown strategy in an established cell line derived from an MMTV-Neu tumor was used (Muraoka et al., 2003). As shown in FIG. 2A, stable expression of two independent siRNA sequences significantly reduced EphA2 expression in MMTV-Neu cells relative to parental cells and cells expressing control siRNA. Pooled populations of cells in which EphA2 expression was diminished displayed slower growth rates than parental or control siRNA-expressing cells (data not shown). Consistent with the diminished growth rates, inhibition of EphA2 expression diminished levels of phosphorylated Erk, a known regulator of proliferation in the MMTV-Neu model [Reviewed in (Eccles, 2001)], in EphA2 siRNA clones (FIG. 2A). Parental MMTV-Neu cells and cells transduced with the control siRNA formed large, multi-acinar structures and failed to form lumens in three-dimensional Matrigel culture, consistent with previous descriptions of the effects of ErbB2 activity on three-dimensional cultures of human MCF10A cells (Muthuswamy et al., 2001). In contrast, diminished EphA2 expression impaired the ErbB2/Neu-driven multi-acinar phenotype of the MMTV-Neu cells in three-dimensional culture. Instead, these cells primarily formed small, organized acini comprised epithelial cells surrounding a central lumen (FIGS. 2B and 2C). Furthermore, the size of individual three-dimensional colonies formed by control cells was 3- to 4-fold higher than cells with decreased EphA2 expression (FIG. 2B). While MMTV-Neu parental cells or cells expressing control siRNAs formed tumors when orthotopically transplanted in the cleared fat pads of FVB recipient female mice, MMTV-Neu cells with diminished EphA2 expression failed to establish tumors or formed very small, non-palpable tumors in a small percentage of animals (FIG. 2D). These data suggest that EphA2 activity is required for tumor cell-intrinsic growth and invasiveness in the context of the ErbB2/Neu oncogene.

Elevated EphA2 Expression Augments Growth and Invasiveness of MCF10A Cells Overexpressing Human ErbB2/HER2

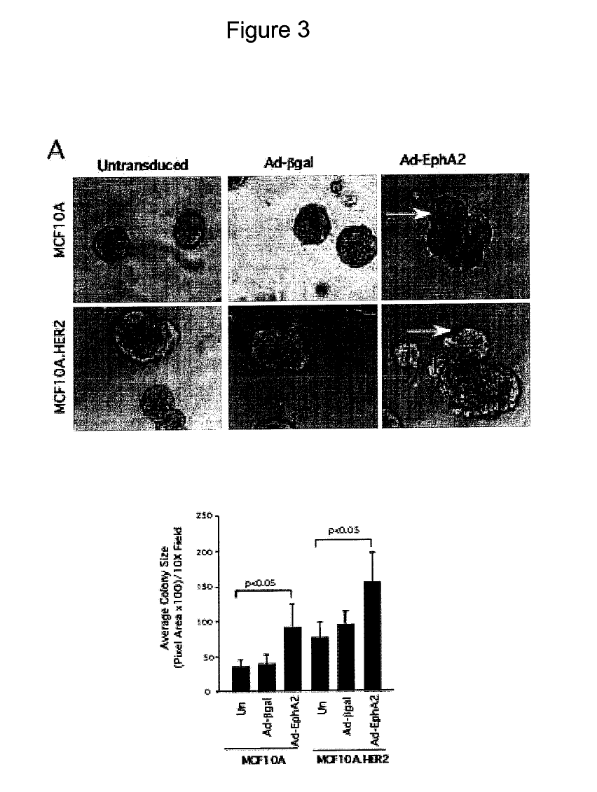

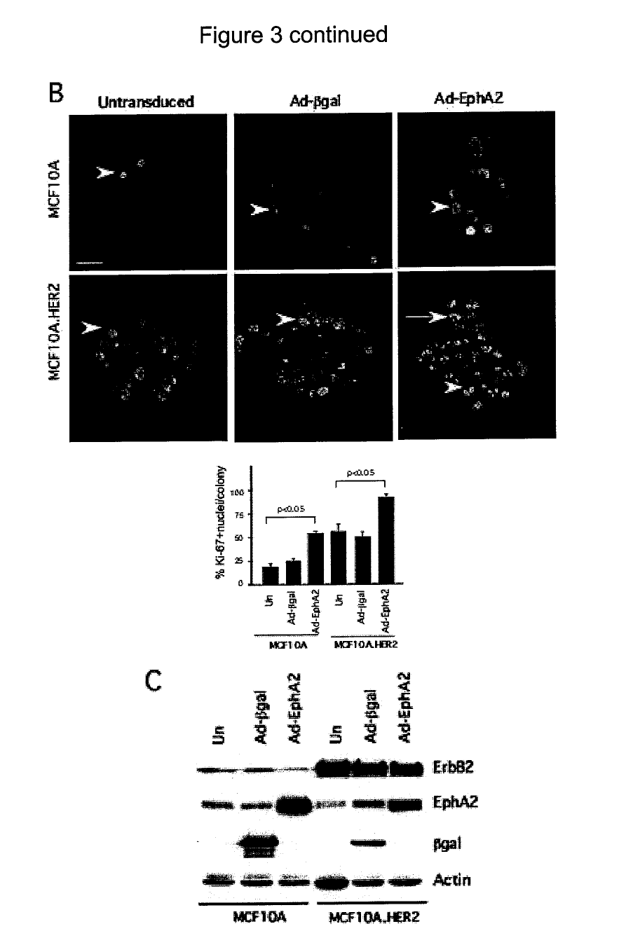

To determine if EphA2 enhances ErbB2-mediated growth and invasiveness, we overexpressed EphA2 in both non-transformed MCF10A human mammary epithelial cells and in MCF10A cells that stably express the human homolog of ErbB2, HER2 (Ueda et al., 2004) by adenoviral transduction. Consistent with previous studies (Zelinski et al., 2001), overexpression of EphA2 enhanced growth, as increased colony size in three-dimensional Matrigel culture was observed (FIG. 3A). Relative to parental MCF10A, HER2-overexpressing cells formed larger, multi-acinar structures which fail to form lumens in three-dimensional Matrigel culture (FIG. 3A), consistent with previous reports (Muthuswamy et al., 2001; Ueda et al., 2004). Overexpression of EphA2 by adenoviral transduction in MCF10A.HER2 cells led to a 2-fold increase in the size of individual colonies relative to untransduced controls or cells transduced with Ad-beta-galactosidase virus (FIG. 3A). In addition, there was an increase in lumen-filling and invasive protrusions in acinar structures formed by MCF10A and MCF10A.HER2 upon overexpression of EphA2, as assessed by confocal microscopy (FIG. 3B). Quantification of nuclear Ki67 revealed that overexpression of EphA2 in MCF10A and MCF10A.HER2 cells increases proliferation nearly 3-fold compared to levels observed in control cells (FIG. 3B). Overexpression of HER2 in MCF10A.HER2 cells, as well as expression of adenoviral gene products, was confirmed by immunoblot (FIG. 4C). These data suggest that EphA2 overexpression is sufficient to promote mammary epithelial proliferation and invasion, and augments growth and invasion induced by ErbB2/HER2.

EphA2 Promotes Activation of Ras/MAPK and Tumor Cell Proliferation

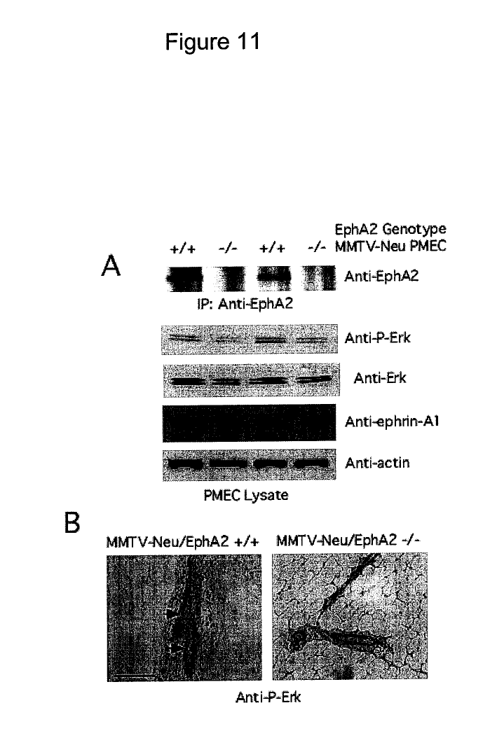

To examine the specific EphA2 signaling events intrinsic to the tumor cells that regulate proliferation, primary MMTV-Neu tumor cells (PMTCs) were purified from EphA2-deficient mice and wild-type controls. EphA2-deficient PMTCs exhibited a decrease in proliferation relative to that in wild-type cells (FIG. 4A), similar to the decrease in proliferation seen in EphA2-deficient PMECs (see FIG. 1E). Decreased proliferation in EphA2-deficient PMTCs was accompanied by diminished phospho-Erk and levels of active GTP-bound Ras (FIG. 4B). EphA2 protein expression in EphA2-deficient tumor cells was not detected (FIG. 4B). However, comparable levels of Neu/ErbB2 protein expression and phosphorylation in tumor cells derived from EphA2-deficient animals versus wild-type controls was observed (FIG. 4B), suggesting that EphA2 does not regulate ErbB2 expression or activity. EphA2 phosphorylation in serum-starved wild-type PMTCs in the absence of ligand stimulation suggests that ErbB2 might regulate the activity of EphA2 (FIG. 4B). Similarly, there was a substantial reduction in Erk and Ras activity in whole tumor lysates from EphA2-deficient animals compared to tumors from wild-type MMTV-Neu mice (FIG. 4C). We also observed diminished basal levels of phosphorylated Erk in serum-starved EphA2-deficient PMEC relative to PMEC isolated from wild-type controls (FIG. 11A). In addition, phospho-Erk was not detectable in EphA2-deficient mammary epithelium by immunohistochemistry, whereas nuclear and cytoplasmic staining was present in control tissue (FIG. 11B). In contrast, we did not detect any significant changes in levels of phospho-src, phospho-stat5, or phospho-PLCg in EphA2-deficient cells relative to wild-type controls under our experimental conditions, suggesting that modulation of Ras/Erk signaling is a primary mechanism through which EphA2 affects Neu-mediated tumor growth. In support of this conclusion, overexpression of exogenous Erk-1 rescued proliferation defects in EphA2-deficient PMTCs relative to cells expressing control b-galactosidase (FIG. 4D). These data suggest that EphA2 promotes tumor cell proliferation upregulation of Erk activity in MMTV-Neu tumor cells.

EphA2 Promotes Tumor Cell Migration Through Activation of RhoA GTPase.

To dissect the mechanisms by which EphA2 promotes tumor metastasis, motility of MMTV-Neu tumor cells in the context of EphA2-deficiency was analyzed using a transwell migration assay. There was a 1.5-fold decrease in the migration of EphA2-deficient MMTV-Neu tumor cells relative to wild-type tumor cells in response to serum stimulation (FIG. 5A). As expression and activity of Rho family small GTPases are integral components of signaling pathways that regulate cell migration, we wished to determine if EphA2 regulates tumor cell motility through a Rho-dependent mechanism. Diminished levels of active GTP-bound RhoA were present in both EphA2-deficient tumors and in purified EphA2-deficient PMTCs relative to wild-type controls (FIG. 5B). EphA2-deficient tumor cells also displayed a decrease in total RhoA protein expression. In contrast, there were no detectable changes in levels of activated Rac1 under our experimental conditions. To determine whether activation of RhoA mediates EphA2-dependent cell migration, we expressed a constitutively active RhoA in EphA2-deficient MMTV-Neu tumor cells. While expression from a control adenovirus expressing b-galactosidase had no effect on migration in EphA2-deficient PMTCs, expression of exogenous activated RhoA restored migration to levels similar to wild-type control cells (FIG. 5C). These findings suggest that RhoA activation contributes to EphA2-mediated tumor cell migration.

Because Rho family GTPases, including RhoA, have also been shown to regulate cell cycle progression (Olson et al., 1995; Welsh et al., 2001), we also investigated whether constitutively active RhoA could rescue proliferation defects in EphA2-deficient tumor cells. Expression of constitutively active RhoA did not rescue proliferation in EphA2-deficient PMTCs, suggesting that RhoA activation specifically contributes to EphA2-mediated tumor cell migration rather than growth. Conversely, to determine whether Ras/MAPK activity could affect cell migration, we overexpressed Erk-1 in EphA2-deficient tumor cells. No change in migration of EphA2-deficient PMTCs upon overexpression of Erk-1 was observed, indicating that regulation of proliferation and motility through independent signaling pathways in the context of ErbB2/EphA2-mediated tumor progression.

EphA2 Physically and Functionally Interacts with ErbB2

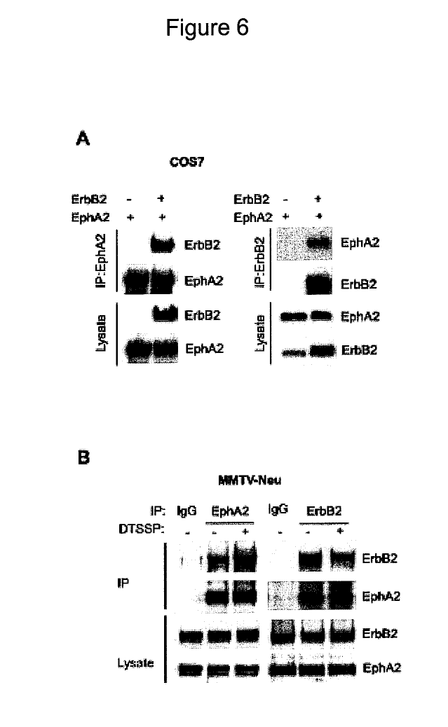

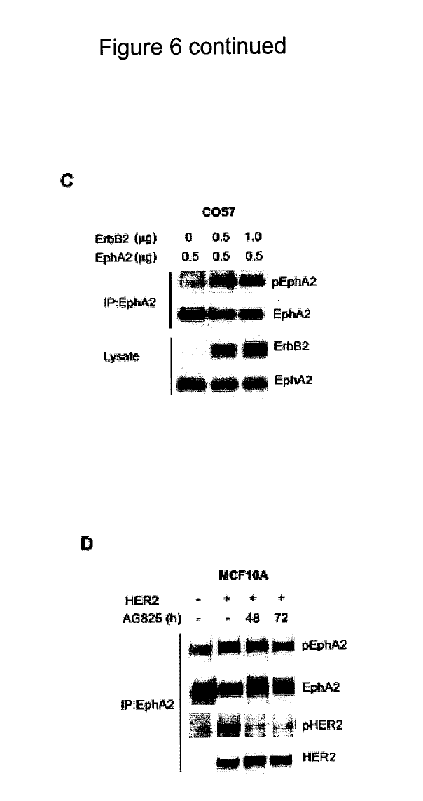

To investigate the molecular mechanism(s) by which EphA2 modulates Neu/ErbB2-mediated proliferation and invasiveness, biochemical studies were performed to assess physical interaction between EphA2 and ErbB2 in COST cells overexpressing both proteins and between endogenous proteins in MMTV-Neu derived primary tumor cells (PMTCs). The presence of ErbB2/Neu in EphA2 immunoprecipitates was detected, as was EphA2 in ErbB2/Neu immunoprecipitates, in lysates from COS7 cells overexpressing the human isoforms of EphA2 and ErbB2 (FIG. 6A). Co-immunoprecipitation analysis of endogenous proteins from PMTCs confirmed that ErbB2 forms a complex with EphA2 (FIG. 6B). In both PMTCs and COS7 cells, EphA2 and ErbB2 were expressed at high levels, and the EphA2/ErbB2 interaction occurred constitutively in the absence of ligand stimulation (FIG. 6C). Strikingly, co-expression of ErbB2 and EphA2 in COS7 cells was sufficient to induce tyrosine phosphorylation of EphA2 in the absence of ligand or serum stimulation (FIG. 6C). Likewise, elevated EphA2 phosphorylation was observed in MCF10A cells overexpressing human HER2/ErbB2 relative to parental MCF10A cells (FIG. 6D). Consistent with co-expression data in COS7 cells, treatment with an ErbB2 kinase inhibitor diminished EphA2 phosphorylation as well as HER2 phosphorylation in MCF10A.HER2 cells (FIG. 6D). Given evidence for physical interaction between ErbB2 and EphA2 and the functional requirement of EphA2 expression for maximal activation of signaling pathways downstream of ErbB2, these data suggest that that EphA2 participates in ErbB2 signaling.

EphA2-Deficiency has No Impact on Tumor Progression, Angiogenesis, or Metastasis in MMTV-PyV-mT Transgenic Animals

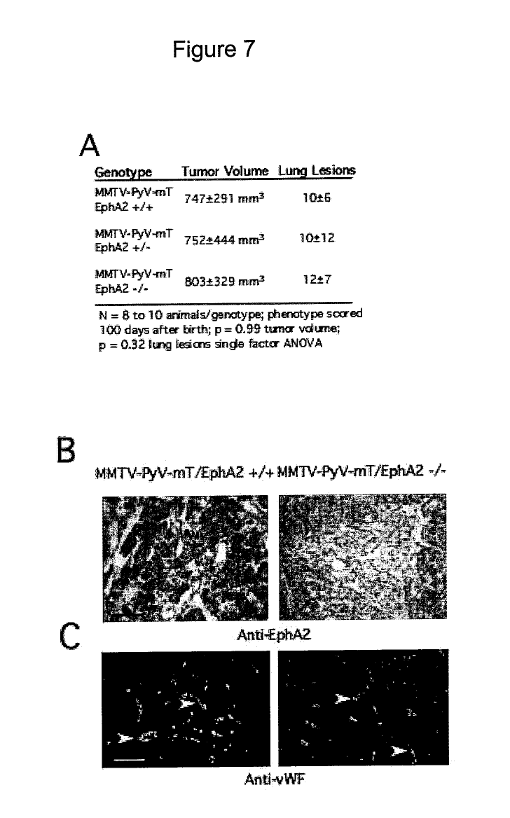

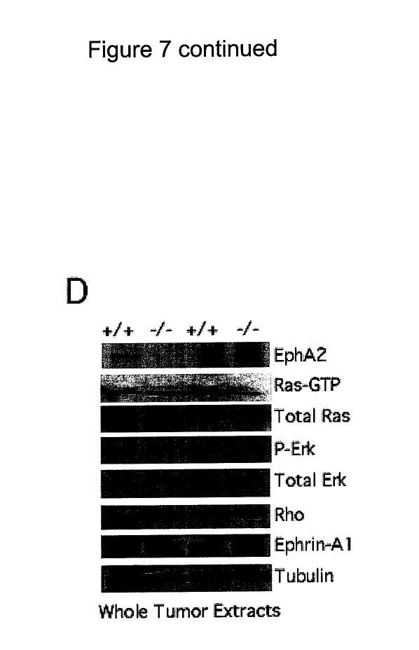

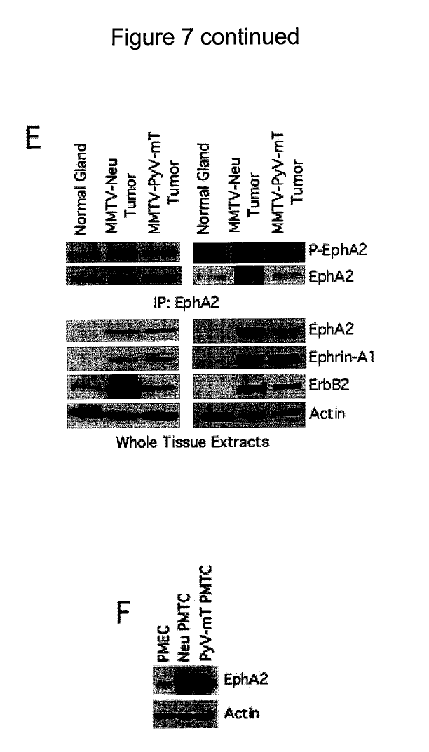

To assess EphA2 function in an independent endogenous model of mammary tumorigenesis that is also dependent upon the Ras/MAPK pathway, MMTV-PyV-mT mice, expressing the polyomavirus middle T antigen under the control of the MMTV promoter, that were wild-type, heterozygous, or EphA2-deficient were generated. Virgin female mice were monitored for tumor formation through 100 days. Despite confirmed loss of EphA2-deficiency in the MMTV-PyV-mT model (FIGS. 7B, D), EphA2-deficiency did not impact rate of tumor formation (data not shown), tumor volume, or the number of surface lung lesions, or microvascular density (FIGS. 7A, C). Additionally, there were no differences in levels of total Ras, active GTP-bound Ras, phospho-Erk, or total Rho in MMTV-PyV-mT tumors derived from wild-type versus EphA2-deficient mice. (FIG. 7D). These findings are in striking contrast to the effects of EphA2-deficiency observed in the MMTV-Neu model. These data suggest that EphA2 does not affect tumorigenesis, progression, and vascularity in the MMTV-PyV-mT model, nor does loss of EphA2 affect the many of the signaling pathways that contribute to these aspects of tumor progression in this model versus similar pathways in the MMTV-Neu model.

Expression and activation of EphA2 in normal mammary tissue isolated from FVB female mice, MMTV-Neu and MMTV-PyV-mT tumor tissue, as well as in PMEC and PMTCs from both MMTV-Neu and MMTV-PyV-mT animals was also assessed. EphA2 is overexpressed and phosphorylated in tumor tissue derived from both MMTV-Neu and MMTV-PyV-mT models compared to normal mammary tissue. Furthermore, expression of ephrin-A1 ligand was elevated in tumor lysates from both models compared to normal mammary tissue, though levels of ephrin-A1 were comparable in wild-type and EphA2-deficient tumor lysates (FIGS. 7E, D). Notably, however, levels of both total and phosphorylated EphA2 were higher in MMTV-Neu tumors compared to MMTV-PyV-mT tumors (FIG. 7E). EphA2 overexpression was detected specifically in tumor epithelium in PMTCs when compared to levels in PMEC (FIG. 7F).

While ErbB2 overexpression has been reported in MMTV-PyV-mT tumors (Lin et al., 2003) and was observed in our tumor lysates, MMTV-Neu tumors displayed a much higher level of ErbB2 overexpression (FIG. 7E). These data suggest that increased expression of EphA2 may be a mechanism by which ErbB2 signaling pathways are amplified in tumors.

Anti-EphA2 Therapy Shows Efficacy in MMTV-Neu Tumor Model

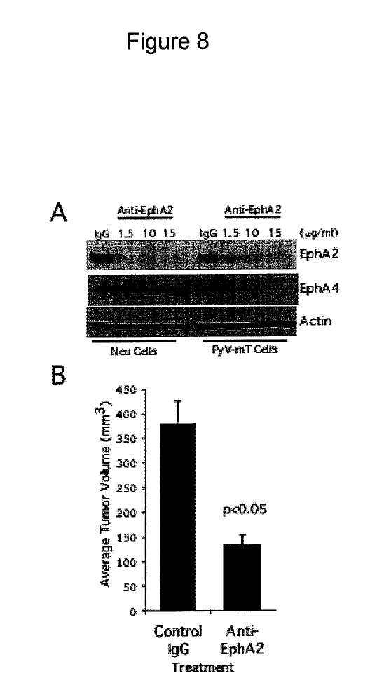

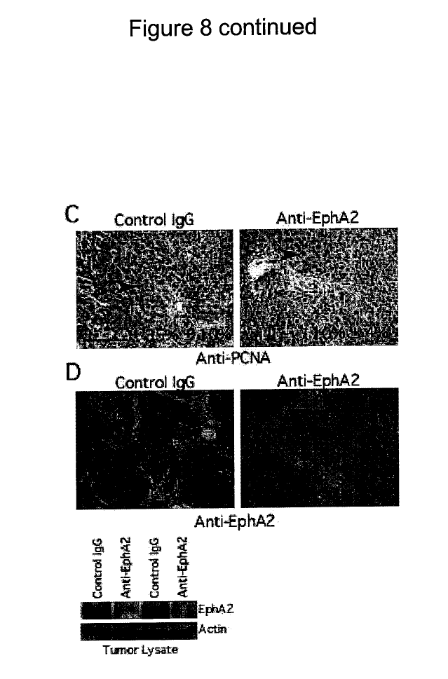

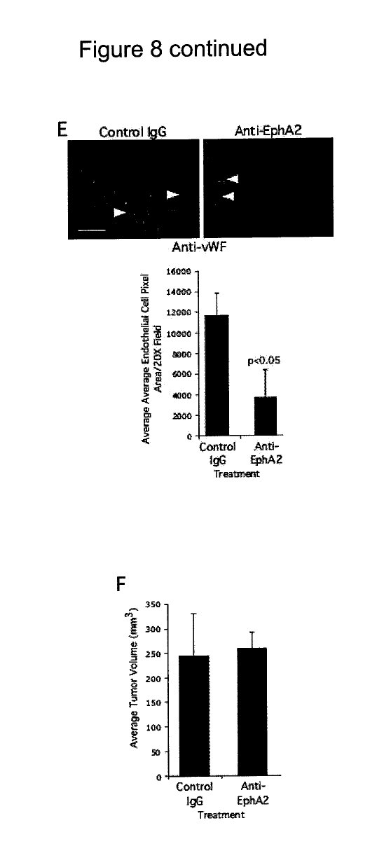

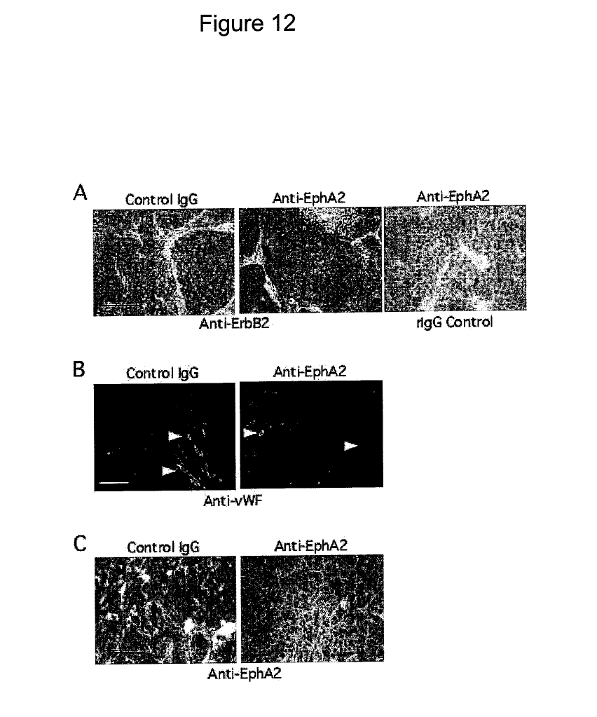

To determine whether MMTV-Neu tumors are responsive to targeted anti-EphA2 therapy in vivo, wild-type Neu tumor cells were transplanted into the cleared fat pads of wild-type FVB recipient animals. Two weeks after transplantation, animals were injected intraperitoneally twice weekly for three weeks with either control IgG or an anti-EphA2 antibody that targets murine EphA2 for degradation (FIG. 8A). The anti-EphA2 antibody specifically targeted EphA2, as expression of the related receptor EphA4 was unaffected in antibody treated tumor cells derived from MMTV-Neu and MMTV-PyV-mT animals (FIG. 8A). MMTV-Neu tumors harvested from anti-EphA2 treated animals displayed a 3-fold reduction in tumor volume relative to tumors isolated from IgG treated mice (FIG. 8B). In addition, tumor cell proliferation was significantly decreased in anti-EphA2 treated animals relative to controls as determined by quantifying nuclear PCNA staining (FIG. 8C). As predicted, EphA2 protein levels are significantly reduced in anti-EphA2-treated tumors relative to control IgG-treated tumors, as assessed by immunohistochemistry and immunoblot (FIG. 8D), though downregulation of EphA2 expression did not affect expression of ErbB2 in anti-EphA2 treated tumors, nor did control IgG treatment affect ErbB2 expression in tumors FIG. 12A). A significant reduction in microvascular density in tumors harvested from anti-EphA2-treated animals relative to those treated with control IgG was observed (FIG. 8E). In contrast to these results, anti-EphA2 treatment had no effect on tumor volume (FIG. 8F) or microvascular density (FIG. 12B) in animals transplanted with MMTV-PyV-mT tumors in spite of downregulated levels of EphA2 protein in anti-EphA2-treated tumors (FIG. 12C). These data suggest that the efficacy of anti-EphA2 therapy depends upon the oncogene context in which tumor progression occurs, as treatment of MMTV-PyV-mT tumor-bearing animals does not impact tumor progression as in MMTV-Neu tumor-bearing mice in spite of EphA2 overexpression in both tumor models.

Whereas, particular embodiments of the invention have been described above for purposes of description, it will be appreciated by those skilled in the art that numerous variations of the details may be made without departing from the invention as described in the appended claims.

All publications, patents and patent applications mentioned in this specification are herein incorporated by reference into the specification to the same extent as if each individual publication, patent or patent application was specifically and individually indicated to be incorporated herein by reference.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

In addition, the following U.S. provisional patent application 60/929,212 filed Jun. 18, 2007 is incorporated by reference in entirety.

In addition, the research article entitled “The receptor tyrosine kinase EphA2 promotes mammary adenocarcinoma tumorigenesis and metastatic progression in mice by amplifying ErbB2 signaling” by Brantley-Sieders, D. M et al. published January 2008 in the Journal of Clinical Investigation, volume 118, number 1 is hereby incorporated by reference in its entirety for all purposes.

1. A method of reducing proliferation of hyperproliferative cells, said method comprising:
a) identifying a population of hyperproliferative cells that express both EphA2 and ErbB2; and
b) administering an agent that targets EphA2.

2. (canceled)

3. (canceled)

4. The method of claim 1, wherein said hyperproliferative cells are cancer cells.

5. The method of claim 4, wherein said cancer is of the skin, lung, colon, breast, prostate, bladder or pancreas, a renal cell carcinoma, a melanoma, a leukemia, or a lymphoma.

6. The method of claim 1, wherein said hyperproliferative cell disease is a non-cancer hyperproliferative cell disease.

7. The method of claim 6, wherein said non-cancer hyperproliferative cell disease is asthma, chronic obstructive pulmonary disease (COPD), psoriasis, lung fibrosis, bronchial hyper responsiveness, seborrheic dermatitis, and cystic fibrosis, inflammatory bowel disease, smooth muscle restenosis, endothelial restenosis, hyperproliferative vascular disease, Behcet's Syndrome, atherosclerosis, or macular degeneration.

8. The method of claim 1, further comprising administering an agent that targets ErbB2.

9. The method of claim 1, wherein said cells overexpress EphA2.

10. The method of claim 1, wherein said cells overexpress ErbB2.

11. The method of claim 1, wherein said cells overexpress both EphA2 and ErbB2.

12. The method of claim 1, wherein said EphA2 targeting agent is agonistic.

13. The method of claim 1, wherein said EphA2 targeting agent is antagonistic.

14. The method of claim 1, wherein said EphA2 or ErbB2 targeting agent is an antibody.

15. The method of claim 1, wherein said EphA2 or ErbB2 targeting agent is a small molecule.

16. The method of claim 1, wherein said EphA2 or ErbB2 targeting agent is a peptide.

17. The method of claim 1, wherein said EphA2 or ErbB2 targeting agent is an siRNA.

18. The method of claim 1, wherein said EphA2 or ErbB2 targeting agent is an antibody-drug candidate (ADC).

19. The method of claim 1, wherein any of said EphA2 or ErbB2 targeting agents inhibits, blocks, or interferes with the interaction between EphA2 and ErbB2.

20. A method of treating a cancer patient, said method comprising:
(a) determining the expression level, presence, or amount of EphA2 and ErbB2 in said patient's cancer cells;
(b) administering an anti-EphA2 and/or an anti-ErbB2 targeting agent if it is determined that said patient's cancer cells express both EphA2 and ErbB2.
